(19) United States
(12) Patent Application Publication
(10) Pub. No.: US 2004/0076622 A1
(43) Pub. Date: Apr. 22, 2004

(54) Local production and/or delivery of anti-cancer agents by stromal cell precursors

(75) Inventors: Matus Studeny, Bratislava (SK); Michael Andreeff, Houston, TX (US); Frank C. Marini, Houston, TX (US)

Correspondence Address: FULBRIGHT & JAWORSKI L.L.P., SUITE 2400, 600 CONGRESS AVENUE, AUSTIN, TX, 78701-3271, US

(73) Assignee: Board of Regents, The University of Texas System

(21) Appl. No.: 10/377,276

(22) Filed: Feb. 28, 2003

Related U.S. Application Data
(60) Provisional application No. 60/361,465, filed on Mar. 2, 2002.

(51) Int. Cl. A61K 48/00; C12N 5/08; C12N 15/86

(52) U.S. Cl. 424/93.21; 435/456; 435/366

(57) ABSTRACT

The present invention concerns the use of compositions comprising, and methods for making, genetically modified mesenchymal stem cells to treat subjects with hyperproliferative disorders. Certain embodiments allow local delivery of an agent while avoiding systemic delivery of the agent alone. Stromat precursor cells may be used to produce a biological agent locally at tumor sites. The tumor microenvironment, or other proliferation inducing microenvironment, preferentially promotes the engraftment of stromal precursors as compared to other tissues.

This application claims the benefit of priority of U.S. Provisional Patent application serial No. 60/361,465 filed on Mar. 2, 2002, the entirety of which is incorporated herein by reference.

The government may own rights in the present invention pursuant to grants number CA55164, CA16672 and CA49639 from the National Institutes of Health.

### BACKGROUND OF THE INVENTION

1. Field of the Invention

The present invention relates generally to the fields of gene therapy, cell biology and cancer therapy. More particularly, it concerns compositions and methods for cell mediated therapy by locally producing and/or delivering an anticancer agent to a tumor.

2. Description of Related Art

Proteins and other biologic agents that control cell growth and proliferation are often produced locally in normal and diseased tissues. Typically, these agents act in a paracrine or autocrine fashion over short physical distances, but are rapidly inactivated and/or degraded as they move away from the site of production, particularly when they reach the circulatory system. This mechanism allows local effects while avoiding unfavorable systemic affects. The sensitivity of these molecules to degradation, or the toxicity of these molecules at elevated levels in the circulation, limits the therapeutic application of these molecules.

One of the difficulties in the treatment of conditions such as cancer, using proteins or other biologic agents, is the need for large quantities of the therapeutic agent to be delivered over an extended period of time. For many of the various peptides, proteins, or compounds discovered during research on diseased cells, tissues, organs or organisms, it has not been possible or commercially feasible to produce the compounds in sufficient quantity to treat the disorders. Numerous examples of these compounds, especially proteins, have been reported.

Therapeutic methods are known that use cell therapies based on administration of genetically modified fibroblast or similar cells into a tumor with the aim of stimulating anti-cancer effects of the immune system. Other methods are based on genetically modified fibroblasts or other cells administered into the body in order to achieve elevated systemic levels of biological agents in vivo. However, none of these methods are entirely satisfactory and, thus, new and improved methodologies are needed.

### SUMMARY OF THE INVENTION

Thus in accordance with the present invention, there are provided compositions comprising stromal cell precursors, mesenchymal stem cells, or precursors thereof, that are genetically modified to produce a therapeutic agent. The production of the therapeutic agent will be localized in an area in, and produced by one or more modified or gene modified cells preferentially localize or where a microenvironment within the body provides for the growth and/or proliferation of the modified or unmodified cells of the invention. Exemplary microenvironments include, but are not limited to a tumor or a wound in a tissue and/or organ, and other proliferative states associated with disease or cellular proliferation. The local production of a therapeutic agent will typically provide an increased local concentration of an agent. In one embodiment, the therapeutic agent is an anti-cancer agent or anti-proliferative agent. An anti-cancer agent includes, but is not limited to a cytokine, a hormone, an extracellular matrix component, an enzyme, a signaling molecule, or an anti-angiogenic polypeptide. In particular embodiments, the therapeutic agent may be interferon-α, interferon-β (IFN-α or IFN-β), MDA7 or the like. The therapeutic agent may be secreted from or expressed on the surface of the genetically modified cells (e.g., stromal cell precursors or mesenchymal stem cells). In certain embodiments, a secreted agent may be produced by the action of an enzyme that is encoded by a gene used to genetically modify a stromal cell precursor, MSC, or precursor thereof. In various embodiments, compositions of the invention will further include a pharmaceutically acceptable carrier. In other embodiments, the agent may be a growth factor or other agent that induces, speeds or otherwise enhanced wound healing.

In certain embodiments an expression vector may be integrated into or associated with a host cell genome. The expression vector may express a therapeutic agent (e.g., IFN-α, IFN-β, nucleic acid encoding an oncolytic virus, etc.) or an enzyme that produces a therapeutic agent. In one embodiment, the expression vector may be integrated into the host cell genome. In other embodiments, the expression vector is maintained episomally within the cell. The expression vector may be a viral expression vector, a plasmid based expression vector, or other known expression system(s).

Various embodiments include methods of treatment for a subject or patient with a disease. In particular embodiments, the subject or patient has been diagnosed with cancer. In various embodiments, the source for cells for genetic modification is the subject being treated, whereas in other embodiments the source for cells for genetic modification is someone other than the subject being treated. In certain embodiments, the method comprises treatment of a subject with cancer including isolating stromal cell precursors or mesenchymal stem cells from a subject; propagating the isolated stromal cell precursors or mesenchymal stem cells in vitro; genetically modifying one or more of the isolated stromal cell precursors or mesenchymal stem cells to express a therapeutic agent; and introducing genetically modified stromal cell precursors or mesenchymal stem cell(s) back into said subject. In other embodiments, genetically modified cells are introduced to a subject by injection. In yet other embodiments, cells that may or may not be genetically modified are introduced by intravascular or intratumoral injection. In certain embodiments, cells of the invention are administered by injection into the carotid artery. Cells of the invention injected into the carotid artery may engraft into brain tumors or other populations of proliferative cells. In particular embodiments, it is contemplated that cells of the invention may engraft in wounds or lesions in the brain caused by variou neurologic disease states or traumatice injury or surgery.

Also provided are methods for the delivery of a therapeutic agent to a cancer cell comprising introducing one or more genetically modified stromal cell precursor, mesenchymal stem cell or precursors thereof to a subject. In certain embodiments, the subject may have, for example, chronic myelogenous leukemia, melanoma, or any other cancer, precancer or proliferative condition. In additional embodiments, the methods described may employ genetically modified stromal cell precursors, stem cells (e.g., mesenchymal stem cells) or a precursor thereof that differentiate into or associates with mesenchymal components of the stroma, as opposed to stem cells that differentiate into hematopoietic cells or cells that do not localize to mesenchymal components of a target area. The cells of the invnetion may also preferentially localize, associate with, and/or engraft into any microenvironment that supports or induces cell proliferation.

Various other embodiments encompass methods that reduce tumor growth, reduce tumor burden, treat metastatic cancer, increase a subjects survival, alleviate symptoms of disease, and/or inhibit a hyperproliferative disease, each achieved by administering compositions of the present invention. In certain embodiments, genetically modified stromal cell precursors, mesenchymal stem cells, or a precursor thereof, that differentiates into, associates with mesenchymal components of the stroma or proliferate in response to a particular environment in the body, are administered by injection. In other embodiments the cells are administered by intravascular or intratumoral injection. In various embodiments the stromal cell precursors or mesenchymal stem cells express INF-α, INF-β and/or MDA7 or other protein that act via a direct or indirect extracellular mechanism or pathway.

Various embodiments include methods of engrafting a therapeutic cell in a tumor including isolating stromal cell precursors, mesenchymal stem cells, or other cell types that localize and engraft in the mesenchymal component of the tumor matrix; propagating the islolated cells in vitro; genetically modifying one or more of the cells so that the cell expresses a therapeutic agent as described herein; and administering the genetically modified cell to a patient or subject. Administration is preferably by intravascular injection.

The use of the word “a” or “an” when used in conjunction with the term “comprising” in the claims and/or the specification may mean “one,” but it is also consistent with the meaning of “one or more,” “at least one,” and “one or more than one.”

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1C illustrates an example of a mesenchymal stem cell (MSC) producing INF-β (INF-β-MSC) that inhibited the growth of A375SM melanoma cells in vitro. A375SM melanoma cells were either cultured alone or co-cultured with un-manipulated MSC and INF-β-MSC for 72 h (FIG. 1A). Numbers of diploid MSC and aneuploid A375SM melanoma cells were determined by flow cytometry (FIG. 1B) and cell counting. INF-β-MSC directly inhibited the growth of A375SM melanoma cells as compared with A375SM cells alone or A375SM cells co-cultured with untransduced MSC (FIG. 1C).

FIGS. 2A-2D illustrates an example of the local production of INF-β by INF-β-MSC in tumors but not systemic levels of INF-D is effective in inhibiting tumor growth in vivo. IFN-β-MSC were either co-injected subcutaneously together with 10⁶ A375SM melanoma cells at the same site or the cells were injected subcutaneously into two separate sites (opposite sides of the animals). Tumor growth was inhibited (FIG. 2A) and the survival of animals prolonged (FIG. 2C) only after the co-injection of A375SM melanoma cells with INF-β-MSC at the same site. Co-injected INF-β-MSC were effective at doses representing 1%, 10% or 50% of the initial malignant cells number. However, systemic levels of INF-β supplied by highest number of INF-β-MSC (50%) injected subcutaneously into a remote site (the side of the animal opposite the tumor) or the subcutaneous administration of a corresponding dose of INF-β (5×10⁴ IU every other day) had no effect. Similarly, the co-injection of melanoma cells with 50% MSC transduced with control adenovirus carrying beta-galactosidase gene (β-Gal) was not effective. Difference in survival was compared by log rank test. Two animals were alive and free of tumors 150 days after cells injection. The tumor size is a mean of 5 animals per group. (FIG. 2D) Survival of mice with A375 metastatic melanomas in their lungs after either the intravenous or subcutaneous injection of INF-β-MSC. Mice were intravenously injected with 10⁶ A375SM melanoma cells and after 10 days started receiving four weekly doses of 10⁶ INF-β-MSC either intravenously (iv) or subcutaneously (sc). Animals were observed until death resulting from melanoma metastasis in the lung. INF-β-MSC injected intravenously produced INF-β locally in lung tumors and this was associated with significantly prolonged survival (P=0.021). Conversely, the systemic levels of INF-β in serum supplied from the same numbers of INF-β-MSC injected subcutanously was not effective (P=0.4). There was a significant difference in the survival of animals with intravenously injected INF-β-MSC as oppose to subcutaneously injected INF-β-MSC (P=0.023). There was no further death after day 100 of the studies and all remaining animals sacrificed at 130 days were tumor-free.

FIG. 3 illustrates an exemplary map of AAVs. The three AAV which produce IFN-α are shown. The AAV-CMA-IFN-α is a constitutive expression cassette. The remaining two AAVs are components of the MFP inducible system. AAV-gal4PRL-65AD expresses the fusion transcription factor (GAL4 and65AD), where as AAV-G5E1b-huIFN contains the chimeric promoter (containing gal4 binding elements), and the human interferon alpha-2B transcription unit.

FIGS. 4A-4B. FIG. 4A Exemplary MFP dependent induction of IFN-α from MSCs. MSCs were infected with both AAV-gal4PRL-65AD, and AAV-G5E1b-huIFN and expanded. After 10 days, cells were split into 12 well dishes, and were fed media containing either MFP (dissolved in 0.1% ETOH) or carrier. Eighteen hours later MFP-containing media was removed and cells washed. Two-hundred microliter samples were removed daily and media was analyzed for human IFN-α expression using the Biosource ELISA IFN-α kit. FIG. 4B illustrated exemplary repeat MFP dependent induction of IFN-α. MSCs from studies in FIG. 4A were monitored to determine baseline levels of IFN-α expression, after baseline was achieved, cells were rested for 5 additional days and then fed media containing MFP (at 10⁻⁸ M) as described above. Samples were removed daily and analyzed for IFN-α production using the Biosource ELISA IFN-α kit. The data shown is the average of duplicate studies.

FIG. 5. MSCs expressing IFN-α inhibit the growth of the CML cell lines K562 and BV173. K562 and BV173 cell lines were grown on feeder layers of MSCs which were infected prior with MFP inducible AAVs and induced with 10⁻⁸ M MFP or grown on MSC layers and fed medium containing 1000 U of Interon A. Aliquots were taken daily and counted for viability (using trypan blue), and cell number. Control wells are CML cell lines grown on MSCs which have not be induced. Data shown reflects three well counted for each point/day ±SEM.

FIG. 6. MSC expressing IFN-α reduce the viability of CML chronic phase CD34+ cells in vitro. Chronic phase CML patient CD34+ cells were magnetically-enriched for CD34 using the Miltenyi AUTOMACS device. Purified CML CD34+ cells were grown on a feeder layer of MSC induced to express IFN-α or an uninfected MSC feeder layer where 1000 U Interon A was added. Cell Viability was assayed using Trypan Blue exclusion. Control wells contained MSC feeder layers but NO MFP or Intron A added. Cell counts were taken daily, and each data point represents three wells counted ±SEM.

FIG. 7 illustrates an example of CML Blasts cells that are growth inhibited when co-cultured on MSCS-IFN expressing feeder layers. Two CML patient samples were Ficoll enriched, and CML Blast cells were added to co-cultures of MSCs either expressing IFN-α, not expressing IFN-α, MSCs infected but not induced or uninfected MSC with exogenously added Interon A (1000 U/ml). Cell counts were assayed on day 3. The data suggests that MSCs induced to express IFN-α as well as adding Interon A is sufficient to inhibit the growth of CML Blast cells in vitro. One interesting point CML Blast cells co-cultured on MSCs feeder layers without IFN-α showed an increase growth, suggesting a positive role in growth for MSC feeder layers.

FIG. 8 illustrates exemplary effects of cell dose on survival of mice after injection of CML cell lines. To determine the dose of cells which results in a reproducible endpoint, K562 or BV173 CML cells were injected iv into mice at three doses (5×10⁶, 1×10⁶, and 5×10⁵) and mice were monitored daily. The data represent the day in which death was noted after cell inoculation for each cell line.

FIG. 9 illustrates an example of systemic expression of IFN-α after im injection. To monitor in vivo expression of IFN-α in BalbC/nu mice after direct intramuscular injection of IFN-α-expressing AAVs. AAV-CMV-IFN-α 1010 G.E./mouse MFP-inducible AAV 5×10¹⁰ G.E./ea/mouse were injected into quadriceps muscles of mice. MFP (6 μg/mouse) was injected IP or given by gavage. Two-hundred microliters of blood was taken weekly, and assayed for IFN-α expression using the BioSource IFN-α Elisa. Results shown are pg/ml of IFN-α detected in the blood.

FIG. 10 illustrates an example of the effect on administration of IFN-MSC i.v. on the metastasis of breast carcinoma MDA 231 in the lungs of SCID mice.

FIG. 11 illustrates an example of MSC-IFNβ administered i.v. inhibits breast carcinoma (MDA 231) metastasis in the lunges of SCID mice.

FIG. 12 illustrates an example of the prolonged survival of mice with metastatic breast carcinoma (MDA 231) treated i.v. with MSC-IFNβ.

FIG. 13 illustrates the plasma levels of IFN-β after administration of IFN-β or MSC-IFNβ into SCID mice.

FIGS. 14A-14C illustrates MSC-IFN-β but not systemically administered IFN-β prolong the survival of mice with MDA 231 or A375SM tumors in lungs. (FIG. 14A) Mice with established pulmonary metastases of MDA 231 carcinoma were intravenously injected with three doses of 10⁶ of MSC-IFN-β or MSC-Gal. An additional group received 100,000 IU IFN-β subcutaneously every other day for the duration of the study. Animals were followed until death. Intravenously injected MSC-IFN-β significantly prolonged survival (P=0.00143) as compared with survival in untreated controls. In contrast, IFN-β or MSC-Gal had no effect on survival (P=0.31 and P=0.51, respectively). (FIG. 14B) Survival of mice with established pulmonary metastasis of A375SM melanoma treated with MSC-IFN-β or daily subcutaneous injections of 40,000 IU IFN-β. IFN-β had no significant effect on survival as compared with survival in untreated group (P=0.06). MSC-IFN-β were administered either intravenously or subcutaneously. Intravenously injected MSC-IFN-β significantly prolonged survival (P=0.0012). In contrast, subcutaneously injected MSC-IFN-β were completely ineffective (P=0.539). This indicates that tumor inhibition was mediated by local effect of MSC-IFN-β that reached the tumors through the bloodstream and engrafted there. In contrast, the systemic level of IFN-β released into the circulation from subcutaneously injected MSC-IFN-β was ineffective.

FIGS. 15A-15C illustrates MSC-Gal engraft in MDA 231 tumors but not in other organs. Three weekly doses of 10⁶ MSC-Gal were injected intravenously in mice with established MDA 231 tumors in lungs (n=5) or in healthy animals (n=5). Mice were sacrificed 14 days after the last dose and 10 slides from each organ examined by X-Gal staining (FIGS. 15A, 15B and 15C). A) MDA 231 tumors in lungs contained numerous colonies of X-Gal positive cells (4±2 colonies per slide, arrows in a). (FIG. 15B) In contrast, only very few single X-Gal positive cells were detected in normal lung (less than 1 cell per slide, arrows in (FIG. 15B). (FIG. 15C) X-Gal positive cells were not detected in spleen, kidney, or muscle and few positive cells were observed in the liver (2±1 cells per slide). This indicates that MSC selectively engrafted in tumor microenvironment but not in the other organs examined.

FIGS. 16A-16F. IFN-β and MSC-IFN-β inhibit proliferation of OVAR-3, SKOV-3, and HEY cells in vitro (FIG. 16A) OVAR-3, (FIG. 16C) SKOV-3, (FIG. 16E) HEY cells were cultured in the presence of increasing concentrations of IFN-β. The effect of IFN-β is expressed as the percentage of the growth of control cells that were not exposed to the IFN-β. Results (mean±SEM) show a concentration-dependent inhibition of cell growth by IFN-β. (FIG. 16B) OVAR-3, (FIG. 16D) SKOV-3, and (FIG. 16F) HEY cells were co-cultured with MSC-βgal or MSC-IFN-β in a 10:1 ratio. Cells were counted, and their relative number in co-cultures was determined by flow cytometry. Results (mean±SEM) are expressed as the percentage of control cells (cultured alone). Growth of cells was significantly inhibited in co-cultures with MSC-IFN-β. These results show that IFN-β and MSC-IFN-β directly inhibit malignant cell growth without the need for an additional component of the immune system for this effect.

FIGS. 17A-17B. Serum levels after the intraperitoneal injection of IFN-β or MSC-IFN-β. (FIG. 17A) Serum levels of IFN-β after the intraperitoneal injection of 40,000 IU of IFN-β. Note the rapid breakdown, to baseline levels within 24 hours. This confirms that recombinant IFN-β cannot sustain systemic levels. (FIG. 17B) Serum levels of IFN-β after the intraperitoneal injection of 5×10⁵ MSC-IFN-β. On the basis of results from ELISA, after infection with 50,000 viral particles per cell Ad IFN-β, 5×10⁵ MSCs produced 40,000 IU of IFN-β in 24 hours. This graph shows that the intraperitoneal injection of MSC-IFN-β can result in detectable levels of IFN-β for at least 6 days, verifying that MSC-IFN-β can sustain IFN-β production/levels in the blood.

FIGS. 18A-18B. Intraperitoneal administration of MSC-IFN-β significantly increases survival in mice with ovarian carcinomas. Mice with established ovarian carcinomas (n=5 for each cell line) were treated with five intraperitoneal injections of 5×10⁵ MSC-IFN-β or MSC-βgal. Additionally, one group received 40,000 IU of IFN-β intraperitoneally every day for 33 days. Control mice did not receive any treatment. (FIG. 18A) Survival curves for OVAR-3 mice. (FIG. 18B) Survival curves for SKOV-3 mice. This indicated that MSC-IFN-β can increase the survival of ovarian carcinoma mice.

FIGS. 19A-19C. MSC- β gal engrafts in tumors but not in other organs. Mice with established ovarian carcinomas (n=5 for each cell line) and normal mice (n=3) were treated with five intraperitoneal injections of 5×10⁵ MSC-βgal. One mouse per group (cell line) was sacrificed when extremely sick, or 14 days after the last dose (normal group). Ten slides of tissue from each organ were examined by X-gal staining. (FIG. 19A) OVAR-3 and (FIG. 19B) SKOV-3 whole tumors contained numerous colonies of X-gal positive cells, as shown by arrows. Slides also contained several colonies, which are shown in ×4 and ×100 magnification. (FIG. 19C) Organs from mice with ovarian carcinomas. X-gal positive cells were not detected in spleen, kidney, muscle, or liver. (d) Slides of tissue from normal mice injected with MSC-βgal. Again, X-gal positive cells were not detected in spleen, kidney, or muscle but a few X-gal positive cells were observed in the liver (2±1 cells per slide). This indicates that MSCs selectively engraft in tumors microenvironment but not in other organs examined.

FIGS. 20A-20B. Growth inhibition of STI-sensitive KBM5 (FIG. 20A) and STI-resistant KBM5/STI cells (FIG. 20B) by STI and IFN-α.

FIG. 21. In vivo studies of MSC-IFN-α.

FIG. 22. Systemic expression of IFN-α after IM injection.

FIG. 23. Growth inhibition of STI-resistant KBM5/STI cells by MDA7-MSC co-cultivation or supernatant derived from MDA7-MSC.

### DESCRIPTION OF ILLUSTRATIVE EMBODIMENTS

One of the various advantages of the present invention is that agents may be expressed at a particular site within the body. Localized production minimizes the distance between the cell producing a therapeutic agent and the target of the therapeutic agent. In addition, local concentration may be elevated to produce therapeutic effects with a reduced toxicity to the organism or patient. Thus, a short-lived agent may be administered to a target cell with minimal degradation or inactivation, although the agent need not be short lived. The present invention may also limit the amount of agent in the systemic circulation or in organ systems.

In various embodiments, the present invention provides methods for delivery or production of a biologic or therapeutic agent(s) at or in site(s) in a body that are associated with cell proliferation and growth factors and other biologic or non-biologic mediators of cell proliferation (e.g., hyperproliferatve conditions such as cancer, wounds, and areas of metastasis). Certain cells isolated from the bone marrow (e.g., stromal cell precursors or mesenchymal stem cells—MSC, this abbreviation may encompass one or more cells) may preferentially engraft and proliferate at sites in an organism that are characterized by increased cell proliferation. Stromal cell precursors or MSC may be maintained in vitro, be genetically modified for therapy purposes, be administered to a subject and be used for disease treatment in vivo. Additionally, non-genetically or genetically modified cells may engraft in or around proliferative, hyperproliferative, cancer or tumors cells and inhibit the proliferative, metastatic or other pathogenic characteristics of proliferating cells. Stromal cell precursors or MSC and genetically modified stromal cell precursors or MSC may be used in the therapeutic methods to inhibit, reduce, or slow the growth of cells involved in a disease state. This approach may be useful not only as a means of improving the pharmacokinetics of various biological or therapeutic agents, but also as a more general tool for modifying the microenvironment in such sites within a body. In other embodiments, various pluripotent, precursor, or stem cells that have the ability to differentiate into, engraft or associate with or within mesenchymal components of a cell, tissue, organ, and/or a cellular or tumor matrix when in or around an appropriate microenvironment, such as a microenvironment in which cell proliferation or hyperproliferation is occurring (e.g., a tumor microenvironment) are contemplated.

Some of the various advantages to the present invention include the ease with which stromal cell precursors or MSC are isolated and propagated. Also, stromal cell precursors or MSC may be efficiently infected in vitro by gene transfer agents, in particular with AAV or adenovirus, however other known gene transfer agents are not excluded. Upon transplant, the therapeutic cell compositions of the present invention may home back or localize to target sites within organs, tissues, tumors and bone marrow. Thus, providing a targeting function for proliferating cell populations. Stromal cell precursors or MSC may engraft in the target tissues and integrate into or around the cellular structure of the organ, tissue, bone marrow, tumor, cancer or target cell population. Stromal cell precursors or MSC may integrate into or around the target and be maintained at the site for extended periods of time. Thus, the cells of the present invention may engraft in the region, location, or area to be treated. The engrafted cells may produce agents for the therapeutic or prophylactic treatment of a disease state, in particular IFN-α and IFN-β.

Embodiments of the invention include compositions comprising, and methods of making and using, genetically modified stromal cell precursors or MSC for the delivery of therapeutic agents. In certain embodiments of the invention, stromal cell precursors or MSCs may be modified to produce biological agent(s) locally at target sites in the body (e.g., tumor sites, bone marrow). A tumor microenvironment will typically promote engraftment of stromal cell precursors or MSC or precursors thereof. In certain embodiments MSCs may be modified in a manner so they express therapeutic agents (e.g., interferon-beta (IFN-β), interferon-alpha (IFN-α) or other therapeutic agents, see below, that inhibit the growth of malignant or hyperproliferative cells. The effect typically requires the integration of stromal cell precursors or MSC into the tumor or surrounding tissue and may not be achieved by systemically delivered biologic agents (e.g., IFN-β or IFN-α). In alternative embodiments, cells other than MSC are contemplated. Other cells that may be used within the scope of the invention include, but are not limited to stromal cell precursors or stem cells in general, embryonic stem cells, neuronal stem cells, or stem cell derived from other tissues, such as placenta, embryo, foreskin, liver, kidney, lung, spleen, intestine, skin, brain, spinal chord, nerve tissue, gonads, and the like. Each of the cell types will have some targeting characteristics unique to that particular stem cell or cell, which may be used advantageously to target diseases derived from different cell lineages. The cell types useful in the practice of the invention will typically engraft in or associate with the mesenchymal components of a proliferating cell population.

Exemplary embodiments, some of which are described in the Examples section below, demonstrate that biological agents may be produced in the locale of modified MSC and produce qualitatively different affects as compared to systemic delivery of the biological agents alone. Thus, stromal cell precursors or MSC may be used as a delivery vehicle for therapeutic agents in the treatment of diseases, for example cancer. In certain embodiments genetically modified stromal cell precursors or MSC may be administered by localized administration. In other embodiments genetically modified stromal cell precursors or MSC may be administered by systemic administration.

Non-limiting examples provided herein, indicate the therapeutic potential of stromal cell precursors or MSC as a delivery system into a tumor microenvironment by their transduction with a therapeutic gene (e.g., human IFN-β gene). Genetically modified stromal cell precursors or MSC may localize in other sites in an organism including, but not limited to locations in and around proliferative and hyperproliferative cells, as well as other sites in the body where stromal cell precursors or MSC are known to localize, such as locations that require supportive mesenchymal stroma, bone marrow, bone fractures, wounds, remodeling tissues and other locations characterized by increased cell turnover.

IFN-α may induce hematological remission in chronic myeloid leukemia (CML) patients, but only a small proportion of patients achieve a sustained, complete cytogenetic remission. Caused primarily by the inability to achieve a high-sustained therapeutic dose of this lymphokine at the proposed active site, the bone marrow. Additionally, patients receiving IFN-α systemically are subjected to debilitating side effects, which prevent constant high doses of this drug, suggesting that local production of controlled high level IFN-α could produce cytogenetic remission, without the systemic side effects. The compositions and methods of the invention may be used as a means of achieving high level sustained expression in a localized manner.

### 1. Stromal Cell Precursors or Mesenchymal Stem Cells (MSC)

Marrow-derived mesenchymal cells are pluripotent cells found in the bone that are capable of differentiating into any of the specific types of connective tissues (i.e., the tissues of the body that support the specialized elements; particularly adipose, areolar, osseous, cartilaginous, elastic, and fibrous connective tissues) depending upon various environmental influences. Embodiments of the present invention are concerned with formation of or association with mesenchymal components of the stroma at a target site using cell types that have been isolated, manipulated and/or genetically modified in vitro. Association or formation of mesenchymal components is influenced by the environment in or around a target site, such as a cancer cell microenvironment. Other cell types, such as stromal cell precursors, may differentiate into or associate with mesenchymal components of stroma. These cell types include precursors to the MSC and other pluripotent cells that have the ability to engraft in or associate with of a mesenchymal component(s) of the stroma. Any cell with ability to form or associate with the cells of the mesenchymal component of the stroma when influenced by the microenvironment associated with proliferating cells (e.g., cancer or tumor microenvironment) may be used in the context of the present invention.

Cells in most, if not all tissues, are hierarchically organized with regard to their proliferative and differentiation potential (Weissman, 2000, incorporated herein by reference). This hierarchy is fully operational in tissues with high spontaneous turnover such as blood, skin and gut. In these tissues, short-lived, terminally differentiated cells are continuously replaced from undifferentiated precursors that are maintained from a compartment of self-renewing stem cells. In contrast, the turnover of connective tissue is low and, its hierarchical organization only become apparent when the demand for new functional cells is increased such as during wound healing or regeneration after injury. Bone marrow-derived mesenchymal stem cells (Fridenshtein et al., 1968; Caplan, 1991) are precursors with a high proliferative capacity (Colter et al., 2000) and can differentiate into adipocytes, chondrocytes, osteoblasts (Pittenger et al., 1999) and possibly other cells types (Woodbury et al., 2000).

Recent data from animal studies and clinical trials indicate that the conditions characterized by increased cell turnover and tissue remodeling such as multiple bone fractures in metabolic bone disease or rapidly growing embryo during prenatal development provide effective signals necessary for survival and proliferation of systemically delivered MSC (Liechty et al., 2000; Horwitz et al., 1999). In a related sense, the proliferation of malignant cells in growing tumors requires formation of supportive mesenchymal stroma (Hanahan and Weinberg, 2000). Process of tumor stroma formation is similar to wound healing (Dvorak, 1986) and result in tissue remodeling with high proliferation of mesenchymal cells (Kuniyasu et aL, 2001). In certain embodiments of the invention, exogenously administered MSC typically would preferentially engraft at the tumor sites and contribute to the population of stromal fibroblasts. Thus, allowing development of therapeutic strategy based the local production of biological agents in tumors by genetically modified MSC. In other embodiments, exogenously administered stromal cell precursors may also preferentially engraft at the tumor sites and contribute to the population of stromal fibroblasts.

Stromal cell precursors or MSC for the methods described herein, can be recovered from other cells in the bone marrow or other mesenchymal stem cell source, for exemplary methods see Deans and Moseley (2000), incorporated herein by reference. Bone marrow cells may be obtained from iliac crest, femora, tibiae, spine, rib or other medullary spaces. Other sources of human mesenchymal stem cells include embryonic yolk sac, placenta, umbilical cord, fetal and adolescent skin, and blood. The presence of mesenchymal stem cells in the culture colonies may be verified by specific cell surface markers which are identified with unique monoclonal antibodies, see, e.g., U.S. Pat. No. 5,486,359. These isolated mesenchymal cell populations display epitopic characteristics associated only with mesenchymal stem cells, have the ability to regenerate in culture without differentiating, and have the ability to differentiate into specific mesenchymal lineages when either induced in vitro or in vivo at a site of damaged tissue.

In certain embodiments, a subpopulation of MSC may be used. Previous reports demonstrated that single-cell derived colonies of MSC contained at least two morphologically distinct cell types: spindle shaped and large flat cells. Recently, a third morphologically distinct MSC cell type has been identified, the small MSC (Colter et al., 2000, incorporated herein by reference). By plating cells at very low density the third morphologically distinct cell type can be detected and isolated. The small MSC cell type is characterized by their extremely small size, rapid rate of replication, and enhanced potential for multilineage differentiation. Also, these cells may be identified by particular surface epitopes and expressed proteins. In certain embodiments, subpopulations of cells may used that have been isolated or enriched for a particular cell population or subpopulation. In alternative embodiments, composition comprising genetically modified small MSC may be used.

Mesenchymal stem cell populations may be isolated from the subject (autologous) or donated by another (heterologous). Any process that is useful in the recovery of MSC from an autologous or heterologous donor may be used to isolate MSC or a population of cells comprising mostly MSC. In one aspect, the method of isolating MSC comprises the steps of providing a tissue sample containing MSC, preferably bone marrow; isolating the MSC from the specimen, for example by density gradient centrifugation; adding the isolated cells to a medium that stimulates MSC growth without differentiation and allows, when cultured, for the selective adherence of the MSC to a substrate surface; culturing the specimen-medium mixture; and removing the non-adherent matter from the substrate surface.

Briefly, bone marrow aspirations or peripheral blood samples are harvested and rinsed once in PBS. The resulting culture is plated on tissue culture plastic in RPMI supplemented with 25% FCS. After 7 days, bone marrow cells are suspended by rubber policeman, and reacted with anti-sh2, sh3, sh4 antibodies (markers for MSCs), after washing, a magnetic microbead reagent is reacted to bind the sh2,3,4 antibodies, and this mixture is passed over a magnetic enrichment column. After 15-18 days, individual colonies grow out which are fibroblast-like in morphology, these are expanded for additional week.

For infection, MSCs are rinsed once with PBS and then incubated with RPMI containing a gene delivery vehicle (e.g., AAV, adenovirus, liposomes). Infection is allowed to proceed. After a specified time interval, fresh media containing 25% FCS is added. Forty-eight hours later cells are analyzed for expression of a control (e.g. x-gal staining for β-gal) or a therapeutic gene of interest (e.g., Immunoblotting blotting). These infected cells are expanded until adequate cell numbers are obtained.

### II. Nucleic Acid-Based Expression Systems

Genetic modification of the cells of the present invention may be accomplished by the uptake and maintenance of nucleic acid-based expression vectors or systems. The expression vectors may be integrated into the host cell genome or maintained episomally.

In various embodiments there are recombinant vectors comprising a DNA segment encoding a therapeutic gene(s). The expression vector, after being transfer to the cell of interest may integrate into a chromosome or be maintained episomally. The term “vector” is used to refer to a carrier nucleic acid molecule into which a nucleic acid sequence can be inserted for introduction into a cell where it can be replicated and/or expressed. A nucleic acid sequence can be “exogenous,” which means that it is foreign to the cell into which the vector is being introduced or that the sequence is homologous to a sequence in the cell but in a position within the host cell nucleic acid in which the sequence is ordinarily not found. Vectors include plasmids, cosmids, viruses (bacteriophage, animal viruses, and plant viruses), and artificial chromosomes (e.g., YACs). One of skill in the art would be well equipped to construct a vector through standard recombinant techniques, which are described in Sambrook et al. (2001) and Ausubel et al (1994), both incorporated herein by reference.

The term “expression vector” refers to any type of genetic construct comprising a nucleic acid coding for a RNA capable of being transcribed. In some cases, RNA molecules are then translated into a protein, polypeptide, or peptide. In other cases, these sequences are not translated, for example, in the production of antisense molecules or ribozymes. Expression vectors can contain a variety of “control sequences,” which refer to nucleic acid sequences necessary for the transcription and possibly translation of an operably linked coding sequence in a particular host cell. In addition to control sequences that govern transcription and translation, vectors and expression vectors may contain nucleic acid sequences that serve other functions as well and are described infra.

### A. Promoters and Enhancers

A “promoter” is a control sequence that is a region of a nucleic acid sequence at which initiation and rate of transcription are controlled. It may contain genetic elements at which regulatory proteins and molecules may bind, such as RNA polymerase and other transcription factors, to initiate the specific transcription a nucleic acid sequence. The phrases “operatively positioned,” “operatively linked,” “under control,” and “under transcriptional control” mean that a promoter is in a correct functional location and/or orientation in relation to a nucleic acid sequence to control transcriptional initiation and/or expression of that sequence.

A promoter generally comprises a sequence that functions to position the start site for RNA synthesis. The best known example of this is the TATA box, but in some promoters lacking a TATA box, such as, for example, the promoter for the mammalian terminal deoxynucleotidyl transferase gene and the promoter for the SV40 late genes, a discrete element overlying the start site itself helps to fix the place of initiation. Additional promoter elements regulate the frequency of transcriptional initiation. Typically, these are located in the region 30-110 bp upstream of the start site, although a number of promoters have been shown to contain functional elements downstream of the start site as well. To bring a coding sequence “under the control of” a promoter, one positions the 5′ end of the transcription initiation site of the transcriptional reading frame “downstream” of (i.e., 3′ of) the chosen promoter. The “upstream” promoter stimulates transcription of the DNA and promotes expression of the encoded RNA.

The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either cooperatively or independently to activate transcription. A promoter may or may not be used in conjunction with an “enhancer,” which refers to a cis-acting regulatory sequence involved in the transcriptional activation of a nucleic acid sequence.

A promoter may be one naturally associated with a nucleic acid sequence, as may be obtained by isolating the 5′ non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other virus, or prokaryotic or eukaryotic cell, and promoters or enhancers not “naturally occurring,” i.e., containing different elements of different transcriptional regulatory regions, and/or mutations that alter expression. For example, promoters that are most commonly used in recombinant DNA construction include the β-lactamase (penicillinase), lactose and tryptophan (trp) promoter systems. In addition to producing nucleic acid sequences of promoters and enhancers synthetically, sequences may be produced using recombinant cloning and/or nucleic acid amplification technology, including PCR™, in connection with the compositions disclosed herein (see U.S. Pat. Nos. 4,683,202 and 5,928,906, each incorporated herein by reference). Furthermore, it is contemplated the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the cell type chosen for expression. Those of skill in the art of molecular biology generally know the use of promoters, enhancers, and cell type combinations for protein expression, (see, for example Sambrook et al. (2001), incorporated herein by reference). The promoters employed may be constitutive, tissue-specific, inducible, and/or useful under the appropriate conditions to direct high level expression of the introduced DNA segment. The promoter may be heterologous or endogenous.

Additionally any promoter/enhancer combination (as per, for example, the Eukaryotic Promoter Data Base EPDB, http://www.epd.isb-sib.ch/) could also be used to drive expression. Use of a T3, T7 or SP6 cytoplasmic expression system is another possible embodiment. Eukaryotic cells can support cytoplasmic transcription from certain bacterial promoters if the appropriate bacterial polymerase is provided, either as part of the delivery complex or as an additional genetic expression construct.

Table A lists non-limiting examples of elements/promoters that may be employed, in the context of the present invention, to regulate the expression of a RNA. Table B provides non-limiting examples of inducible elements, which are regions of a nucleic acid sequence that can be activated in response to a specific stimulus.

TABLE A
Promoter and/or Enhancer

| Promoter/Enhancer | References |
|---|---|
| Immunoglobulin Heavy Chain | Banerji et al., 1983; Gilles et al., 1983; Grosschedl |
| | et al., 1985; Atchinson et al., 1986, 1987; Imler et |
| | al., 1987; Weinberger et al., 1984; Kiledjian et al., |
| | 1988; Porton et al.; 1990 |
| Immunoglobulin Light Chain | Queen et al., 1983; Picard et al., 1984 |
| T-Cell Receptor | Luria et al., 1987; Winoto et al., 1989; Redondo et |
| | al.; 1990 |
| HLA DQ a and/or DQ β | Sullivan et al., 1987 |
| β-Interferon | Goodbourn et al., 1986; Fujita et al., 1987; |
| | Goodbourn et al., 1988 |
| Interleukin-2 | Greene et al., 1989 |
| Interleukin-2 Receptor | Greene et al., 1989; Lin et al., 1990 |
| MHC Class II 5 | Koch et al., 1989 |
| MHC Class II HLA-Dra | Sherman et al., 1989 |
| β-Actin | Kawamoto et al., 1988; Ng et al; 1989 |
| Muscle Creatine Kinase (MCK) | Jaynes et al., 1988; Horlick et al., 1989; Johnson et |
| | al., 1989 |
| Prealbumin (Transthyretin) | Costa et al., 1988 |
| Elastase I | Ornitz et al., 1987 |
| Metallothionein (MTII) | Karin et al., 1987; Culotta et al., 1989 |
| Collagenase | Pinkert et al., 1987; Angel et al., 1987 |
| Albumin | Pinkert et al., 1987; Tronche et al., 1989, 1990 |
| α-Fetoprotein | Godbout et al., 1988; Campere et al., 1989 |
| γ-Globin | Bodine et al., 1987; Perez-Stable et al., 1990 |
| β-Globin | Trudel et al., 1987 |
| c-fos | Cohen et al., 1987 |
| c-HA-ras | Triesman, 1986; Deschamps et al., 1985 |
| Insulin | Edlund et al., 1985 |
| Neural Cell Adhesion Molecule | Hirsch et al., 1990 |
| (NCAM) | |
| α₁-Antitrypsin | Latimer et al., 1990 |
| H2B (TH2B) Histone | Hwang et al., 1990 |
| Mouse and/or Type I Collagen | Ripe et al., 1989 |
| Glucose-Regulated Proteins | Chang et al., 1989 |
| (GRP94 and GRP78) | |
| Rat Growth Hormone | Larsen et al., 1986 |
| Human Serum Amyloid A (SAA) | Edbrooke et al., 1989 |
| Troponin I (TN I) | Yutzey et al., 1989 |
| Platelet-Derived Growth Factor | Pech et al., 1989 |
| (PDGF) | |
| Duchenne Muscular Dystrophy | Klamut et al., 1990 |
| SV40 | Banerji et al., 1981; Moreau et al., 1981; Sleigh et |
| | al., 1985; Firak et al., 1986; Herr et al., 1986; Imbra |
| | et al., 1986; Kadesch et al., 1986; Wang et al., |
| | 1986; Ondek et al., 1987; Kuhl et al., 1987; |
| | Schaffner et al., 1988 |
| Polyoma | Swartzendruber et al., 1975; Vasseur et al., 1980; |
| | Katinka et al., 1980, 1981; Tyndell et al., 1981; |
| | Dandolo et al., 1983; de Villiers et al., 1984; Hen et |
| | al., 1986; Satake et al., 1988; Campbell and/or |
| | Villarreal, 1988 |
| Retroviruses | Kriegler et al., 1982, 1983; Levinson et al., 1982; |
| | Kriegler et al., 1983, 1984a, b, 1988; Bosze et al., |
| | 1986; Miksicek et al., 1986; Celander et al., 1987; |
| | Thiesen et al., 1988; Celander et al., 1988; Choi et |
| | al., 1988; Reisman et al., 1989 |
| Papilloma Virus | Campo et al., 1983; Lusky et al., 1983; Spandidos |
| | and/or Wilkie, 1983; Spalholz et al., 1985; Lusky et |
| | al., 1986; Cripe et al., 1987; Gloss et al., 1987; |
| | Hirochika et al., 1987; Stephens et al., 1987 |
| Hepatitis B Virus | Bulla et al., 1986; Jameel et al., 1986; Shaul et al., |
| | 1987; Spandau et al., 1988; Vannice et al., 1988 |
| Human Immunodeficiency Virus | Muesing et al., 1987; Hauber et al., 1988; |
| | Jakobovits et al., 1988; Feng et al., 1988; Takebe et |
| | al., 1988; Rosen et al., 1988; Berkhout et al., 1989; |
| | Laspia et al., 1989; Sharp et al., 1989; Braddock et |
| | al., 1989 |
| Cytomegalovirus (CMV) | Weber et al., 1984; Boshart et al., 1985; Foecking |
| | et al., 1986 |
| Gibbon Ape Leukemia Virus | Holbrook et al., 1987; Quinn et al., 1989 |

TABLE B
Inducible Elements

| Element | Inducer | References |
|---|---|---|
| MT II | Phorbol Ester (TFA) | Palmiter et al., 1982; |
| | Heavy metals | Haslinger et al., 1985; |
| | | Searle et al., 1985; Stuart et |
| | | al., 1985; Imagawa et al., |
| | | 1987, Karin et al., 1987; |
| | | Angel et al., 1987b; |
| | | McNeall et al., 1989 |
| MMTV (mouse | Glucocorticoids | Huang et al., 1981; Lee et |
| mammary | | al., 1981; Majors et al., |
| tumor virus) | | 1983; Chandler et al., |
| | | 1983; Lee et al., 1984; |
| | | Ponta et al., 1985; Sakai et |
| | | al., 1988 |
| β-Interferon | Poly(rI)x | Tavernier et al., 1983 |
| | Poly(rc) | |
| Adenovirus 5 E2 | E1A | Imperiale et al., 1984 |
| Collagenase | Phorbol Ester (TPA) | Angel et al., 1987a |
| Stromelysin | Phorbol Ester (TPA) | Angel et al., 1987b |
| SV40 | Phorbol Ester (TPA) | Angel et al., 1987b |
| Murine MX Gene | Interferon, Newcastle | Hug et al., 1988 |
| | Disease Virus | |
| GRP78 Gene | A23187 | Resendez et al., 1988 |
| α-2-Macroglobulin | IL-6 | Kunz et al., 1989 |
| Vimentin | Serum | Rittling et al., 1989 |
| MHC Class I Gene | Interferon | Blanar et al., 1989 |
| H-2 κb HSP70 | E1A, SV40 Large T | Taylor et al., 1989, 1990a, |
| | Antigen | 1990b |
| Proliferin | Phorbol Ester-TPA | Mordacq et al., 1989 |
| Tumor Necrosis | PMA | Hensel et al., 1989 |
| Factor α | | |
| Thyroid Stimulating | Thyroid Hormone | Chatterjee et al., 1989 |
| Hormone α Gene | | |

The identity of tissue-specific promoters or elements, as well as assays to characterize their activity, is well known to those of skill in the art. Nonlimiting examples of such regions include the human LIMK2 gene (Nomoto et al. 1999), the somatostatin receptor 2 gene (Kraus et al., 1998), murine epididymal retinoic acid-binding gene (Lareyre et al., 1999), human CD4 (Zhao-Emonet et al., 1998), mouse alpha2 (XI) collagen (Tsumaki, et al., 1998), D1A dopamine receptor gene (Lee, et al., 1997), insulin-like growth factor II (Wu et al., 1997), and human platelet endothelial cell adhesion molecule-1 (Almendro et al., 1996).

### B. Initiation Signals and Internal Ribosome Binding Sites

A specific initiation signal also may be required for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

In certain embodiments of the invention, the use of internal ribosome entry sites (IRES) elements are used to create multigene, or polycistronic, messages. IRES elements are able to bypass the ribosome scanning model of 5′ methylated Cap dependent translation and begin translation at internal sites (Pelletier and Sonenberg, 1988). IRES elements from two members of the picomavirus family (polio and encephalomyocarditis) have been described (Pelletier and Sonenberg, 1988), as well an IRES from a mammalian message (Macejak and Sarnow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message (see U.S. Pat. Nos. 5,925,565 and 5,935,819, each herein incorporated by reference).

### C. Multiple Cloning Sites

Vectors can include a multiple cloning site (MCS), which is a nucleic acid region that contains multiple restriction enzyme sites, any of which can be used in conjunction with standard recombinant technology to digest the vector (see, for example, Carbonelli et al., 1999, Levenson et al., 1998, and Cocea, 1997, incorporated herein by reference.) “Restriction enzyme digestion” refers to catalytic cleavage of a nucleic acid molecule with an enzyme that functions only at specific locations in a nucleic acid molecule. Many of these restriction enzymes are commercially available. Use of such enzymes is widely understood by those of skill in the art. Frequently, a vector is linearized or fragmented using a restriction enzyme that cuts within the MCS to enable exogenous sequences to be ligated to the vector. “Ligation” refers to the process of forming phosphodiester bonds between two nucleic acid fragments, which may or may not be contiguous with each other. Techniques involving restriction enzymes and ligation reactions are well known to those of skill in the art of recombinant technology.

### D. Splicing Sites

Most transcribed eukaryotic RNA molecules will undergo RNA splicing to remove introns from the primary transcripts. Vectors containing genomic eukaryotic sequences may require donor and/or acceptor splicing sites to ensure proper processing of the transcript for protein expression (see, for example, Chandler et al., 1997, herein incorporated by reference.)

### E. Termination Signals

The vectors or constructs of the present invention will generally comprise at least one termination signal. A “termination signal” or “terminator” is comprised of the DNA sequences involved in specific termination of an RNA transcript by an RNA polymerase. Thus, in certain embodiments a termination signal that ends the production of an RNA transcript is contemplated. A terminator may be necessary in vivo to achieve desirable message levels.

In eukaryotic systems, the terminator region may also comprise specific DNA sequences that permit site-specific cleavage of the new transcript so as to expose a polyadenylation site. This signals a specialized endogenous polymerase to add a stretch of about 200 A residues (polyA) to the 3′ end of the transcript. RNA molecules modified with this polyA tail appear to more stable and are translated more efficiently. Thus, in other embodiments involving eukaryotes, it is preferred that that terminator comprises a signal for the cleavage of the RNA, and it is more preferred that the terminator signal promotes polyadenylation of the message. The terminator and/or polyadenylation site elements can serve to enhance message levels and to minimize read through from the cassette into other sequences.

Terminators contemplated for use in the invention include any known terminator of transcription described herein or known to one of ordinary skill in the art, including but not limited to, for example, the termination sequences of genes, such as for example the bovine growth hormone terminator or viral termination sequences, such as for example the SV40 terminator. In certain embodiments, the termination signal may be a lack of transcribable or translatable sequence, such as due to a sequence truncation.

### F. Polyadenylation Signals

In expression, particularly eukaryotic expression, one will typically include a polyadenylation signal to effect proper polyadenylation of the transcript. The nature of the polyadenylation signal is not believed to be crucial to the successful practice of the invention, and any such sequence may be employed. Preferred embodiments include the SV40 polyadenylation signal or the bovine growth hormone polyadenylation signal, convenient and known to function well in various target cells. Polyadenylation may increase the stability of the transcript or may facilitate cytoplasmic transport.

### G. Origins of Replication

In order to propagate a vector in a host cell, it may contain one or more origins of replication sites (often termed “ori”), which is a specific nucleic acid sequence at which replication is initiated. Alternatively an autonomously replicating sequence (ARS) can be employed if the host cell is yeast.

### H. Selectable and Screenable Markers

In certain embodiments of the invention, cells containing a nucleic acid construct of the present invention may be identified in vitro or in vivo by including a marker in the expression vector. Such markers would confer an identifiable change to the cell permitting easy identification of cells containing the expression vector. Generally, a selectable marker is one that confers a property that allows for selection. A positive selectable marker is one in which the presence of the marker allows for its selection, while a negative selectable marker is one in which its presence prevents its selection. An example of a positive selectable marker is a drug resistance marker.

Usually the inclusion of a drug selection marker aids in the cloning and identification of transformants, for example, genes that confer resistance to neomycin, puromycin, hygromycin, DHFR, GPT, zeocin and histidinol are useful selectable markers. In addition to markers conferring a phenotype that allows for the discrimination of transformants based on the implementation of conditions, other types of markers including screenable markers such as GFP, whose basis is colorimetric analysis, are also contemplated. Alternatively, screenable enzymes such as herpes simplex virus thymidine kinase (tk) or chloramphenicol acetyltransferase (CAT) may be utilized. One of skill in the art would also know how to employ immunologic markers, possibly in conjunction with FACS analysis. The marker used is not believed to be important, so long as it is capable of being expressed simultaneously with the nucleic acid encoding a gene product. Further examples of selectable and screenable markers are well known to one of skill in the art.

### I. Plasmid Vectors

In certain embodiments, a plasmid vector is contemplated for use to transform a host cell. In general, plasmid vectors containing replicon and control sequences which are derived from species compatible with the host cell are used in connection with these hosts. The vector ordinarily carries a replication site, as well as marking sequences which are capable of providing phenotypic selection in transformed cells. In a non-limiting example, E. coli is often transformed using derivatives of pBR322, a plasmid derived from an E. coli species. pBR322 contains genes for ampicillin and tetracycline resistance and thus provides easy means for identifying transformed cells. The pBR plasmid, or other microbial plasmid or phage must also contain, or be modified to contain, for example, promoters which can be used by the microbial organism for expression of its own proteins.

In addition, phage vectors containing replicon and control sequences that are compatible with the host microorganism can be used as transforming vectors in connection with these hosts. For example, the phage lambda GEM™-11 may be utilized in making a recombinant phage vector which can be used to transform host cells, such as, for example, E. coli LE392.

Further useful plasmid vectors include pIN vectors (Inouye and Inouye, 1985); and pGEX vectors, for use in generating glutathione S-transferase (GST) soluble fusion proteins for later purification and separation or cleavage. Other suitable fusion proteins are those with β-galactosidase, ubiquitin, and the like.

Bacterial host cells, for example, E. coli, comprising the expression vector, are grown in any of a number of suitable media, for example, LB. The expression of the recombinant protein in certain vectors may be induced, as would be understood by those of skill in the art, by contacting a host cell with an agent specific for certain promoters, e.g., by adding IPTG to the media or by switching incubation to a higher temperature. After culturing the bacteria for a further period, generally of between 2 and 24 h, the cells are collected by centrifugation and washed to remove residual media.

### J. Viral Vectors

The ability of certain viruses to infect cells or enter cells via receptor-mediated endocytosis, and to integrate into host cell genome and express viral genes stably and efficiently have made them attractive candidates for the transfer of foreign nucleic acids into cells (e.g., mammalian cells). Non-limiting examples of virus vectors that may be used to deliver a nucleic acid of the present invention are described below.

### 1. AAV Vectors

The nucleic acid may be introduced into the cell using adenovirus assisted transfection. Increased transfection efficiencies have been reported in cell systems using adenovirus coupled systems (Kelleher and Vos, 1994; Cotten et al., 1992; Curiel, 1994). Adeno-associated virus (AAV) is an attractive vector system for use in the present invention as it has a high frequency of integration and it can infect nondividing cells, thus making it useful for delivery of genes into mammalian cells, for example, in tissue culture (Muzyczka, 1992) or in vivo. AAV has a broad host range for infectivity (Tratschin et al., 1984; Laughlin et al., 1986; Lebkowski et al., 1988; McLaughlin et al., 1988). Details concerning the generation and use of rAAV vectors are described in U.S. Pat. Nos. 5,139,941 and 4,797,368, each incorporated herein by reference.

### 2. Adenoviral Vectors

A particular method for delivery of the nucleic acid involves the use of an adenovirus expression vector. Although adenovirus vectors are known to have a low capacity for integration into genomic DNA, this feature is counterbalanced by the high efficiency of gene transfer afforded by these vectors. “Adenovirus expression vector” is meant to include those constructs containing adenovirus sequences sufficient to (a) support packaging of the construct and (b) to ultimately express a tissue or cell-specific construct that has been cloned therein. Knowledge of the genetic organization or adenovirus, a 36 kb, linear, double-stranded DNA virus, allows substitution of large pieces of adenoviral DNA with foreign sequences up to 7 kb (Grunhaus and Horwitz, 1992).

### 3. Retroviral Vectors

Retroviruses have promise as delivery vectors for the genetic modification in the methods described herein due to their ability to integrate their genes into the host genome, transferring a large amount of foreign genetic material, infecting a broad spectrum of species and cell types and of being packaged in special cell-lines (Miller, 1992).

In order to construct a retroviral vector, a nucleic acid (e.g., one encoding an therapeutic gene of interest) is inserted into the viral genome in the place of certain viral sequences to produce a virus that is replication-defective. In order to produce virions, a packaging cell line containing the gag, pol, and env genes but without the LTR and packaging components is constructed (Mann et al., 1983). When a recombinant plasmid containing a cDNA, together with the retroviral LTR and packaging sequences is introduced into a special cell line (e.g., by calcium phosphate precipitation for example), the packaging sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is then collected, optionally concentrated, and used for gene transfer. Retroviral vectors are able to infect a broad variety of cell types. However, integration and stable expression require the division of host cells (Paskind et al., 1975).

Lentiviruses are complex retroviruses, which, in addition to the common retroviral genes gag, pol, and env, contain other genes with regulatory or structural function. Lentiviral vectors are well known in the art (see, for example, Naldini et al., 1996; Zufferey et al., 1997; Blomer et al., 1997; U.S. Pat. Nos. 6,013,516 and 5,994,136). Some examples of lentivirus include the Human Immunodeficiency Viruses: HIV-1, HIV-2 and the Simian Immunodeficiency Virus: SIV. Lentiviral vectors have been generated by multiply attenuating the HIV virulence genes, for example, the genes env, vif, vpr, vpu and nef are deleted making the vector biologically safe.

Recombinant lentiviral vectors are capable of infecting non-dividing cells and can be used for both in vivo and ex vivo gene transfer and expression of nucleic acid sequences. For example, recombinant lentivirus capable of infecting a non-dividing cell wherein a suitable host cell is transfected with two or more vectors carrying the packaging functions, namely gag, pol and env, as well as rev and tat is described in U.S. Pat. No. 5,994,136, incorporated herein by reference. One may target the recombinant virus by linkage of the envelope protein with an antibody or a particular ligand for targeting to a receptor of a particular cell-type. By inserting a sequence (including a regulatory region) of interest into the viral vector, along with another gene which encodes the ligand for a receptor on a specific target cell, for example, the vector is now target-specific.

### 4. Other Viral Vectors

Other viral vectors may be employed as nucleic acid constructs and genetic modification methods in the present invention. Vectors derived from viruses such as vaccinia virus (Ridgeway, 1988; Baichwal and Sugden, 1986; Coupar et al., 1988), sindbis virus, cytomegalovirus and herpes simplex virus may be employed. They offer several attractive features for various mammalian cells (Friedmann, 1989; Ridgeway, 1988; Baichwal and Sugden, 1986; Coupar et al., 1988; Horwich et al., 1990).

### 5. Delivery Using Modified Viruses

A nucleic acid to be delivered may be housed within an infective virus that has been engineered to express a specific binding ligand. The virus particle will thus bind specifically to the cognate receptors of the target cell and deliver the contents to the cell. A novel approach designed to allow specific targeting of retrovirus vectors was developed based on the chemical modification of a retrovirus by the chemical addition of lactose residues to the viral envelope. This modification can permit the specific infection of hepatocytes via sialoglycoprotein receptors.

Another approach to targeting of recombinant retroviruses was designed in which biotinylated antibodies against a retroviral envelope protein and against a specific cell receptor were used. The antibodies were coupled via the biotin components by using streptavidin (Roux et al, 1989). Using antibodies against major histocompatibility complex class I and class II antigens, they demonstrated the infection of a variety of human cells that bore those surface antigens with an ecotropic virus in vitro (Roux et al., 1989).

### B. Vector Delivery and Cell Transformation

Suitable methods for nucleic acid delivery for transformation of a cell for use with the current invention are believed to include virtually any method by which a nucleic acid (e.g., DNA) can be introduced into a cell, as described herein or as would be known to one of ordinary skill in the art. Such methods include, but are not limited to, direct delivery of DNA such as by ex vivo transfection (Wilson et al., 1989; Nabel et al., 1989), by electroporation (U.S. Pat. No. 5,384,253, incorporated herein by reference; Tur-Kaspa et al., 1986; Potter et al., 1984); by calcium phosphate precipitation (Graham and Van Der Eb, 1973; Chen and Okayama, 1987; Rippe et al., 1990); by using DEAE-dextran followed by polyethylene glycol (Gopal, 1985); by direct sonic loading (Fechheimer et al., 1987); by liposome mediated transfection (Nicolau and Sene, 1982; Fraley et al., 1979; Nicolau et al., 1987; Wong et al., 1980; Kaneda et al., 1989; Kato et al., 1991) and receptor-mediated transfection (Wu and Wu, 1987; Wu and Wu, 1988); and any combination of such methods. Through the application of techniques such as these, cell(s) may be stably or transiently transformed.

### 1. Ex Vivo Transformation

Methods for transfecting cells removed from an organism in an ex vivo setting are known to those of skill in the art. For example, canine endothelial cells have been genetically altered by retroviral gene transfer in vitro and transplanted into a canine (Wilson et al., 1989). In another example, yucatan minipig endothelial cells were transfected by retrovirus in vitro and transplanted into an artery using a double-balloon catheter (Nabel et al., 1989). Thus, it is contemplated that cells or tissues may be removed and transfected ex vivo using the nucleic acids of the present invention. In particular aspects, the cells may be placed into an organism.

### 2. Electroporation

In certain embodiments of the present invention, a nucleic acid is introduced into an organelle, a cell, a tissue or an organism via electroporation. Electroporation involves the exposure of a suspension of cells and DNA to a high-voltage electric discharge. In some variants of this method, certain cell wall-degrading enzymes, such as pectin-degrading enzymes, are employed to render the target recipient cells more susceptible to transformation by electroporation than untreated cells (U.S. Pat. No. 5,384,253, incorporated herein by reference). Alternatively, recipient cells can be made more susceptible to transformation by mechanical wounding.

Transfection of eukaryotic cells using electroporation has been quite successful. Mouse pre-β lymphocytes have been transfected with human kappa-immunoglobulin genes (Potter et al., 1984), and rat hepatocytes have been transfected with the chloramphenicol acetyltransferase gene (Tur-Kaspa et al., 1986) in this manner.

### 3. Calcium Phosphate

In other embodiments of the present invention, a nucleic acid is introduced to the cells using calcium phosphate precipitation. Human KB cells have been transfected with adenovirus 5 DNA (Graham and Van Der Eb, 1973) using this technique. Also in this manner, mouse L(A9), mouse C127, CHO, CV-1, BHK, NIH3T3 and HeLa cells were transfected with a neomycin marker gene (Chen and Okayama, 1987), and rat hepatocytes were transfected with a variety of marker genes (Rippe et al., 1990).

### 4. DEAE-Dextran

In another embodiment, a nucleic acid is delivered into a cell using DEAE-dextran followed by polyethylene glycol. In this manner, reporter plasmids were introduced into mouse myeloma and erythroleukemia cells (Gopal, 1985).

### 5. Sonication Loading

Additional embodiments of the present invention include the introduction of a nucleic acid by direct sonic loading. LTK⁻ fibroblasts have been transfected with the thymidine kinase gene by sonication loading (Fechheimer et al., 1987).

### 6. Liposome-Mediated Transfection

In a further embodiment of the invention, a nucleic acid may be entrapped in a lipid complex such as, for example, a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). Also contemplated is an nucleic acid complexed with Lipofectamine (Gibco BRL) or Superfect (Qiagen).

Liposome-mediated nucleic acid delivery and expression of foreign DNA in vitro has been very successful (Nicolau and Sene, 1982; Fraley et al., 1979; Nicolau et al., 1987). The feasibility of liposome-mediated delivery and expression of foreign DNA in cultured chick embryo, HeLa and hepatoma cells has also been demonstrated (Wong et al, 1980).

In certain embodiments of the invention, a liposome may be complexed with a hemagglutinating virus (HVJ). This has been shown to facilitate fusion with the cell membrane and promote cell entry of liposome-encapsulated DNA (Kaneda et al., 1989). In other embodiments, a liposome may be complexed or employed in conjunction with nuclear non-histone chromosomal proteins (HMG-1) (Kato et al., 1991). In yet further embodiments, a liposome may be complexed or employed in conjunction with both HVJ and HMG-1. In other embodiments, a delivery vehicle may comprise a ligand and a liposome.

### 7. Receptor Mediated Transfection

Still further, a nucleic acid may be delivered to a target cell via receptor-mediated delivery vehicles. These take advantage of the selective uptake of macromolecules by receptor-mediated endocytosis that will be occurring in a target cell. In view of the cell type-specific distribution of various receptors, this delivery method adds another degree of specificity to the present invention.

Certain receptor-mediated gene targeting vehicles comprise a cell receptor-specific ligand and a nucleic acid-binding agent. Others comprise a cell receptor-specific ligand to which the nucleic acid to be delivered has been operatively attached. Several ligands have been used for receptor-mediated gene transfer (Wu and Wu, 1987; Wagner et al., 1990; Perales et al., 1994; Myers, EPO 0273085), which establishes the operability of the technique. Specific delivery in the context of another mammalian cell type has been described (Wu and Wu, 1993; incorporated herein by reference). In certain aspects of the present invention, a ligand will be chosen to correspond to a receptor specifically expressed on the target cell population.

In other embodiments, a nucleic acid delivery vehicle component of a cell-specific nucleic acid targeting vehicle may comprise a specific binding ligand in combination with a liposome. The nucleic acid(s) to be delivered are housed within the liposome and the specific binding ligand is functionally incorporated into the liposome membrane. The liposome will thus specifically bind to the receptor(s) of a target cell and deliver the contents to a cell. Such systems have been shown to be functional using systems in which, for example, epidermal growth factor (EGF) is used in the receptor-mediated delivery of a nucleic acid to cells that exhibit upregulation of the EGF receptor.

In still further embodiments, the nucleic acid delivery vehicle component of a targeted delivery vehicle may be a liposome itself, which will preferably comprise one or more lipids or glycoproteins that direct cell-specific binding. For example, lactosyl-ceramide, a galactose-terminal asialganglioside, have been incorporated into liposomes and observed an increase in the uptake of the insulin gene by hepatocytes (Nicolau et al., 1987). It is contemplated that the tissue-specific transforming constructs of the present invention can be specifically delivered into a target cell in a similar manner.

### 8. Microprojectile Bombardment

Microprojectile bombardment techniques can be used to introduce a nucleic acid into at least one, organelle, cell, tissue or organism (U.S. Pat. Nos. 5,550,318; 5,538,880; and 5,610,042; and PCT Application WO 94/09699; each of which is incorporated herein by reference). This method depends on the ability to accelerate DNA-coated microprojectiles to a high velocity allowing them to pierce cell membranes and enter cells without killing them (Klein et al., 1987). There are a wide variety of microprojectile bombardment techniques known in the art, many of which are applicable to the invention.

In this microprojectile bombardment, one or more particles may be coated with at least one nucleic acid and delivered into cells by a propelling force. Several devices for accelerating small particles have been developed. One such device relies on a high voltage discharge to generate an electrical current, which in turn provides the motive force (Yang et al., 1990). The microprojectiles used have consisted of biologically inert substances such as tungsten or gold particles or beads. Exemplary particles include those comprised of tungsten, platinum, and preferably, gold. It is contemplated that in some instances DNA precipitation onto metal particles would not be necessary for DNA delivery to a recipient cell using microproj ectile bombardment. However, it is contemplated that particles may contain DNA rather than be coated with DNA. DNA-coated particles may increase the level of DNA delivery via particle bombardment but are not, in and of themselves, necessary.

For the bombardment, cells in suspension are concentrated on filters or solid culture medium. The cells to be bombarded are positioned at an appropriate distance below the macroprojectile stopping plate.

An illustrative embodiment of a method for delivering DNA into a cell (e.g., a plant cell) by acceleration is the Biolistics Particle Delivery System, which can be used to propel particles coated with DNA or cells through a screen, such as a stainless steel or Nytex screen, onto a filter surface covered with cells. The screen disperses the particles so that they are not delivered to the recipient cells in large aggregates. It is believed that a screen intervening between the projectile apparatus and the cells to be bombarded reduces the size of projectiles aggregate and may contribute to a higher frequency of transformation by reducing the damage inflicted on the recipient cells by projectiles that are too large.

### C. Host Cells

Host cells of the invention include stromal cell precursors or mesenchymal stem cells as well as progenitors, precursors, or other stem cells that engraft in or associate with the mesenchymal components of target sites of the invention. As used herein, the terms “cell,” “cell line,” and “cell culture” may be used interchangeably. All of these terms also include their progeny, which is any and all subsequent generations. It is understood that all progeny may not be identical due to deliberate or inadvertent mutations. In the context of expressing a heterologous nucleic acid sequence encoding a therapeutic agent, “host cell” refers to an eukaryotic cell, and it includes any transformable cell that is capable of replicating a vector and/or expressing a heterologous gene encoded by a vector. A host cell can, and has been, used as a recipient for vectors. A host cell may be “transfected” or “transformed,” which refers to a process by which exogenous nucleic acid is transferred or introduced into the host cell. A transformed cell includes the primary subject cell and its progeny. As used herein, the tenns “engineered” “genetically modified”, “genetically altered” and “recombinant” cells or host cells are intended to refer to a cell into which an exogenous nucleic acid sequence, such as, for example, a vector, has been introduced. Therefore, recombinant cells are distinguishable from naturally occurring cells which do not contain a recombinantly introduced nucleic acid. In various embodiments of the invention, host cells may be one or more of stem cells, precursors of stem cells, or stem that have undergone at least some physiologic changes resulting in some degree of differentiation. In certain embodiments host cells may be MSC or precursors thereof.

In certain embodiments, it is contemplated that RNAs or proteinaceous sequences may be co-expressed with other selected RNAs or proteinaceous sequences in the same host cell. Co-expression may be achieved by co-transfecting the host cell with two or more distinct recombinant vectors. Alternatively, a single recombinant vector may be constructed to include multiple distinct coding regions for RNAs, which could then be expressed in host cells transfected with the single vector.

A tissue may be part of or separated from an organism. In certain embodiments, a tissue may comprise, but is not limited to, adipocytes, alveolar, ameloblasts, axon, basal cells, blood, lymphocytes, blood vessel, bone, bone marrow, brain, breast, cartilage, cervix, colon, cornea, embryonic, endometrium, endothelial, epithelial, esophagus, facia, fibroblast, follicular, ganglion cells, glial cells, goblet cells, kidney, liver, lung, lymph node, muscle, neuron, ovaries, pancreas, peripheral blood, prostate, skin, skin, small intestine, spleen, stem cells, stomach, testes, anthers, ascite tissue, cobs, ears, flowers, husks, kernels, leaves, meristematic cells, pollen, root tips, roots, silk, stalks, and all cancers thereof.

In certain embodiments, the host cell or tissue may be comprised in at least one organism. In certain embodiments, the organism may be, but is not limited to, a prokayote (e.g., a eubacteria, an archaea) or an eukaryote, as would be understood by one of ordinary skill in the art (see, for example, webpage http://phylogeny.arizona.edu/tree/phylogeny.html).

Numerous cell lines and cultures are available for use as a host cell, and they can be obtained through the American Type Culture Collection (ATCC), which is an organization that serves as an archive for living cultures and genetic materials (www.atcc.org) or may be primary culture obtained from tissues samples derived from a subject in need to treatment or a donor subject that is not the subject in need of treatment. An appropriate host can be determined by one of skill in the art based on the vector backbone and the desired result. A plasmid or cosmid, for example, can be introduced into a prokaryote host cell for replication of many vectors. Cell types available for vector replication and/or expression include, but are not limited to, bacteria, such as E. coli (e.g., E. coli strain RR1, E. coli LE392, E. coli B, E. coli X 1776 (ATCC No. 31537) as well as E. coli W3110 (F-, lambda-, prototrophic, ATCC No. 273325), DH5α, JM109, and KC8, bacilli such as Bacillus subtilis; and other Enterobacteriaceae such as Salmonella typhimurium, Serratia marcescens, various Pseudomonas specie, as well as a number of commercially available bacterial hosts such as SURE® Competent Cells and SOLOPACK™ Gold Cells (STRATAGENE™, La Jolla). In certain embodiments, bacterial cells such as E. coli LE392 are particularly contemplated as host cells for phage viruses.

Examples of eukaryotic host cells for replication and/or expression of a vector include, but are not limited to, HeLa, NIH3T3, Jurkat, 293, Cos, CHO, Saos, and PC12. Many host cells from various cell types and organisms are available and would be known to one of skill in the art. Similarly, a viral vector may be used in conjunction with either a eukaryotic or prokaryotic host cell, particularly one that is permissive for replication or expression of the vector. In various embodiments of the invention stem cells are used as a host cell and in certain embodiments MSC are used as host cells.

Some vectors may employ control sequences that allow it to be replicated and/or expressed in both prokaryotic and eukaryotic cells. One of skill in the art would further understand the conditions under which to incubate all of the above described host cells to maintain them and to permit replication of a vector. Also understood and known are techniques and conditions that would allow large-scale production of vectors, as well as production of the nucleic acids encoded by vectors and their cognate polypeptides, proteins, or peptides.

### III Therapeutic Genes

Therapeutic genes expressed by genetically modified cells of the present invention may be used in the therapeutic or prophylactic treatment of diseases, such as cancer, and other proliferative conditions. The therapeutic genes may have a direct effect on a cell of interest and/or initiate, stimulate or enhance biological processes of the body, such as an immune response.

In the embodiments of the invention, various classes of therapeutic genes may be used. Therapeutic genes may include, but are not limited to cytokines, hormones, toxins, extracellular matrix components, enzymes, cell surface molecules, therapeutically active peptides (e.g. angiostatin) and the like.

### A. Cytokines

A class of biologic modifiers that is contemplated to be used in the present invention includes interleukins and cytokines, such as interleukin 1 (IL-1), IL-2, IL-3, IL-4, IL-5, IL-6, IL-7, IL-8, IL-9, IL-10, IL-11, IL-12, IL-13, IL-14, IL-15, INF-α, INF-β, γ-interferon, angiostatin, thrombospondin, endostatin, METH-1, METH-2, Flk2/Flt3 ligand, GM-CSF, G-CSF, M-CSF, and tumor necrosis factor (TNF).

Interferons (IFNs), are soluble proteins that originally were found to induce antiviral activity in target cells. IFNs have been since known to inhibit cell division and modulate the immune response. IFN-alpha produces an overall response rate of 20% in advanced melanoma and is associated with a 42% improvement in the fraction of patients with high risk melanoma who are disease-free.

In various embodimetns of the invention the melanoma differentiation associated protein 7 (MDA7) is specifically contemplated as being used to modify a stromal cell precursor or an MSC. An MDA7-MSC or stromoal cell precusure may be utilized in the various methods described herein. MDA7 was identified following treatment of melanoma cells with interferon-α and mezerin, Jiang and Fisher noted loss of proliferative ability and terminal differentiation (Jiang et al, 1996). Jiang and Fisher developed a novel subtraction hybridization scheme in human melanoma cells and this resulted in the identification and cloning of a series of melanoma-differentiation-associated (MDA) genes implicated in growth-controlled differentiation and apoptosis. One of the MDA genes identified, MDA7, was noted to be a novel gene and expression of this gene correlated with the induction of terminal differentiation in human melanoma cells (Jiang et al., 1996; Jiang et al., 1995). The MDA7 gene was noted to be expressed at high levels in proliferating normal melanocytes, but the expression was decreased as disease progressed to metastatic disease. Jiang et al. (1995 and 1996), subsequently demonstrated that, when MDA7 was expressed in a wide variety of tumor cells, this resulted in growth suppression and apoptosis. This has subsequently been confirmed by several additional groups. In addition, several groups have confirmed that the MDA7 gene effectively induces cell death in tumor cells with no significant toxicity to normal cells (Saeki et al., 2000; Saeki et al., 2002). The MDA7 gene was recently mapped to chromosome 1q32, an area containing a cluster of genes associated with the IL-10 family of cytokines (Mhashilkar et al., 2001). MDA7 has now been classified as interleukin-24 and has been demonstrated to bind to the IL-20 and IL-22 receptors, and subsequently mediate cell signaling. Because of its potent antitumor activity and the apparent selectivity for cancer cells without toxicity to normal cells, this gene has been proposed as a novel tumor suppressor gene that may be effective in the treatment of cancer.

### B. Hormones

Additional embodiments embrace the use of a hormone as a biologic modifier. For example, the following hormones or steroids can be implemented in the present invention: prednisone, progesterone, estrogen, androgen, gonadotropin, ACTH, CGH, or gastrointestinal hormones such as secretin.

### C. Toxins

In certain embodiments of the present invention, therapeutic agents will include generally a plant-, fungus-, or bacteria-derived toxin such as ricin A-chain (Burbage, 1997), a ribosome inactivating protein, a-sarcin, aspergillin, restrictocin, a ribonuclease, diphtheria toxin A (Masuda et al., 1997; Lidor, 1997), pertussis toxin A subunit, E. coli enterotoxin toxin A subunit, cholera toxin A subunit, and pseudomonas toxin c-terminal. Recently, it was demonstrated that transfection of a plasmid containing a fusion protein regulatable diphtheria toxin A chain gene was cytotoxic for cancer cells. Thus, gene transfer of regulated toxin genes might also be applied to the treatment of diseases (Masuda et al., 1997).

### D. Chemokines

Chemokines also may be used in the present invention. Chemokines generally act as chemoattractants to recruit immune effector cells to the site of chemokine expression. It may be advantageous to express a particular chemokine gene in combination with, for example, a cytokine gene, to enhance the recruitment of other immune system components to the site of treatment. Such chemokines include RANTES, MCAF, MIP1-alpha, MIP1-beta, and IP-10. The skilled artisan will recognize that certain cytokines are also known to have chemoattractant effects and could also be classified under the term chemokines.

### E. Cell Cycle Regulators

Cell cycle regulators provide possible advantages, when combined with other genes. Such cell cycle regulators include p27, p16, p21, p57, p18 , p73 , p19, p15, E2F-1, E2F-2, E2F-3, p107, p130 and E2F-4. Other cell cycle regulators include anti-angiogenic proteins, such as soluble Flt1 (dominant negative soluble VEGF receptor), soluble Wnt receptors, soluble Tie2/Tek receptor, soluble hemopexin domain of matrix metalloprotease 2 and soluble receptors of other angiogenic cytokines (e.g., VEGFR1/KDR, VEGFR3/Flt4, both VEGF receptors).

### F. Inducers of Apoptosis

Inducers of apoptosis, such as Bax , Bak, Bcl-Xs , Bad , Bim, Bik, Bid, Harakiri, Ad E1B, Bad, ICE-CED3 proteases, TRAIL, SARP-2 and apoptin, similarly could find use according to the present invention.

### G. Tumor Suppressors

Tumor suppressors may also be employed according to the present invention and include, but are not limited to p53, p16, CCAM, p21, p15, BRCA1, BRCA2, IRF-1, PTEN (MMAC1), RB, APC, DCC, NF-1, NF-2, WT-1, MEN-I, MEN-II, zac1, p73, VHL, FCC, MCC, DBCCR1, DCP4 and p57.

### H. Single Chain Antibodies

In yet another embodiment, therapeutic agents may comprise a single-chain antibody. Methods for the production of single-chain antibodies are well known to those of skill in the art. The skilled artisan is referred to U.S. Pat. No. 5,359,046, (incorporated herein by reference) for such methods. A single chain antibody is created by fusing together the variable domains of the heavy and light chains using a short peptide linker, thereby reconstituting an antigen binding site on a single molecule.

Single-chain antibody variable fragments (scFvs) in which the C-terminus of one variable domain is tethered to the N-terminus of the other via a 15 to 25 amino acid peptide or linker, have been developed without significantly disrupting antigen binding or specificity of the binding (Bedzyk et al, 1990; Chaudhary et aL., 1990). These Fvs lack the constant regions (Fc) present in the heavy and light chains of the native antibody.

Antibodies to a wide variety of molecules are contemplated, such as oncogenes, growth factors, hormones, enzymes, transcription factors or receptors. Also contemplated are secreted antibodies, targeted to serum, against angiogenic factors (VEGF/VSP, βFGF, αFGF and others) and endothelial antigens necessary for angiogenesis (i.e., V3 integrin). Specifically contemplated are growth factors such as transforming growth factor and platelet derived growth factor.

It may be advantageous to combine portions of genomic DNA with cDNA or synthetic sequences to generate specific constructs. For example, where an intron is desired in the ultimate construct, a genomic clone will need to be used. The cDNA or a synthesized polynucleotide may provide more convenient restriction sites for the remaining portion of the construct and, therefore, would be used for the rest of the sequence.

Particular oncogenes that are targets for antisense constructs are ras, myc, neu, raf, erb, src, fms, jun, trk , ret, hst, gsp, bcl-2 and abl. Also contemplated to be useful will be anti-apoptotic genes and angiogenesis promoters.

As described herein, it is contemplated that any one particular gene may be combined with any other particular gene.

### I. Cytolytic or Oncolytic Viruses

In certain embodiments, a genetically modified cell may produce a cytolytic or oncolytic virus. The cell will typically localize in a tumor microenvironment where virus produced by the modified cell will generally infect the surrounding cells. In certain embodiments the virus will selectively or preferentially lyse or kill hyperproliferative or tumor cells. Cytolytic or oncolytic viruses are known. Examples of oncolytic viruses include mutated adenovirus (Heise et al., 1997), mutated vaccinia virus (Gnant et al., 1999) and mutated reovirus (Coffey et al., 1998). Examples of viral vectors for use in gene therapy include mutated vaccinia virus (Lattime et al., 1996), mutated herpes simplex virus (Toda et al., 1998), mutated adenovirus (U.S. Pat. No. 5,698,443) and mutated retroviruses (Anderson, 1998), each of which is incorporated herein by reference.

### J. Combined Therapy

In many therapies, it will be advantageous to provide more than one functional therapeutic. Such “combined” therapies may have particular import in treating multiple aspects of condition, disease, or other abnormal physiology. For example, treating multidrug resistant (MDR) cancers. Thus, one aspect of the present invention utilizes a genetically modified stem cell to deliver therapeutic compounds to an appropriate site in a tissue, organ or organism for treatment of diseases, while a second therapy, either targeted or non-targeted, is also provided.

A non-targeted treatment may precede or follow genetically modified stem cell treatment by intervals ranging from minutes to weeks. In embodiments where the other agent and genetically modified stem cells are administered separately to the site of interest, one would generally ensure that a significant period of time did not expire between the time of each delivery, such that the agent and the genetically modified stem cell would still be able to exert an advantageously combined effect on a treatment site. In such instances, it is contemplated that one would contact the cell with both modalities within about 12-24 h of each other and, more preferably, within about 6-12 h of each other, with a delay time of only about 12 h being most preferred. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

It also is conceivable that more than one administration of either agent will be desired. Various combinations may be employed, where the genetically modified stem cell agent is “A” and the other agent is “B”, as exemplified below:

A/B/A B/A/B B/B/A A/A/B B/A/A A/B/B B/B/B/A B/B/A/B
A/A/B/B A/B/A/B A/B/B/A B/B/A/A B/A/B/A B/A/A/B B/B/B/A
A/A/A/B B/A/A/A A/B/A/A A/A/B/A A/B/B/B B/A/B/B B/B/A/B

Other combinations are contemplated. For example, in the context of the present invention, it is contemplated that genetically modified stem cells of the present invention could be used in conjunction with non-targeted anti-cancer agents, including chemo- or radiotherapeutic intervention. To kill cells, inhibit cell growth, inhibit metastasis, inhibit angiogenesis or otherwise reverse or reduce the malignant phenotype of tumor cells, using the methods and compositions of the present invention, one would generally contact a “target” cell with a genetically modified stem cell agent, as described herein and at least one other agent; these compositions would be provided in a combined amount effective achieve these goals. This process may involve exposing the site(s) targeted for treatment with the genetically modified stem cells and an other agent(s) or factor(s) at the same time. This may be achieved by administering a single composition or pharmacological formulation that includes both agents, or by administering two distinct compositions or formulations, at the same time, wherein one composition includes a genetically modified stem cell and another includes the other agent.

Agents or factors suitable for use in a combined therapy are any chemical compound or treatment method with therapeutic activity. For example, an “anticancer agent” refers to an agent with anticancer activity. These compounds or methods include alkylating agents, topisomerase I inhibitors, topoisomerase II inhibitors, RNA/DNA antimetabolites, DNA antimetabolites, antimitotic agents, as well as DNA damaging agents, which induce DNA damage when applied to a cell.

Examples of alkylating agents include, inter alia, chloroambucil, cis-platinum, cyclodisone, flurodopan, methyl CCNU, piperazinedione, teroxirone. Topisomerase I inhibitors encompass compounds such as camptothecin and camptothecin derivatives, as well as morpholinodoxorubicin. Doxorubicin, pyrazoloacridine, mitoxantrone, and rubidazone are illustrations of topoisomerase II inhibitors. RNA/DNA antimetabolites include L-alanosine, 5-fluoraouracil, aminopterin derivatives, methotrexate, and pyrazofurin; while the DNA antimetabolite group encompasses, for example, ara-C, guanozole, hydroxyurea, thiopurine. Typical antimitotic agents are colchicine, rhizoxin, taxol, and vinblastine sulfate. Other agents and factors include radiation and waves that induce DNA damage such as, γ-irradiation, X-rays, UV-irradiation, microwaves, electronic emissions, and the like. A variety of anti-cancer agents, also described as “chemotherapeutic agents,” function to induce DNA damage, all of which are intended to be of use in the combined treatment methods disclosed herein. Chemotherapeutic agents contemplated to be of use, include, e.g., adriamycin, bleomycin, 5-fluorouracil (5FU), etoposide (VP-16), camptothecin, actinomycin-D, mitomycin C, cisplatin (CDDP), podophyllotoxin, verapamil, and even hydrogen peroxide. The invention also encompasses the use of a combination of one or more DNA damaging agents, whether radiation-based or actual compounds, such as the use of X-rays with cisplatin or the use of cisplatin with etoposide.

The skilled artisan is directed to “Remington's Pharmaceutical Sciences” 15th Edition, Chapter 33, in particular pages 624-652. Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

In certain embodiments of the invention local delivery of a therapeutic agent by a genetically modified stem cell in patients with cancers, precancers, or hyperproliferative conditions will typically be directed to a site interest by the preferential localization of the stems cells. Similarly, the chemo- or radiotherapy may be directed to a particular, affected region of a subjects body. Alternatively, systemic delivery of compounds and/or the agents may be appropriate in certain circumstances, for example, where extensive metastasis has occurred.

In addition to combining genetically modified stem cell therapies with chemo- and radiotherapies, it also is contemplated that combination with gene therapies will be advantageous. For example, using a combination of p53, p16, p21, Rb, APC, DCC, NF-1, NF-2, BCRA2, p16, FHIT, WT-1, MEN-I, MEN-II, BRCA1, VHL, FCC, or MCC, or antisense versions of the oncogenes ras, myc, neu, raf, erb, src, fms, jun, trk, ret, gsp, hst, bcl, abl, or any of the genes mentioned above are included within the scope of the invention.

### V. Disease States

The present invention deals with the treatment of disease states that involve hyperproliferative disorders including hepatitis and an the like, benign and malignant neoplasias. Such disorders include hematological malignancies, hepatitis, restenosis, cancer, multi-drug resistant cancer, primary, psoriasis, inflammatory bowel disease, rheumatoid arthritis, osteoarthritis and metastatic tumors.

In particular, the present invention is directed at the treatment of human cancers including cancers of the prostate, lung, brain, glioma, neurobalstoma, skin, liver, breast, lymphoid system, multiple myelomas, lyphomas, stomach, testicular, ovarian, pancreatic, bone, bone marrow, head and neck, cervical, esophagus, eye, gall bladder, kidney, adrenal glands, heart, colon, rectum and blood. Other diseases that may be treated with compositions or methods of the invention also may include renal cell carcinomas; viral infections such as, hepatitis C (Garini et al., 2001), HIV-1 (Hatzakis et al., 2001); Erdheim-Chester disease (Esmali et al., 2001), thrombocytopenic purpura (Dikici et al., 2001), marburg hemorrhagic fever (Kolokol'tsov et al., 2001) In certain embodiments, methods and composition are used to treat a subject with CML. In other embodiments, methods and compositions of the invention are used to treat a subject with melanoma.

In certain embodiments, the cells of the invention may be used to repair damaged tissue such neurons, liver, kidney and any other organ or tissue of the body.

### A Chronic Myelogenous Leukemia (CML)

CML arises from a clonal expansion of transformed hematopoietic stem cells capable of differentiation into mature granulocytic cells. The Philadelphia chromosome (Ph) is a hallmark of the disease in which the reciprocal translocation t(9:22) results in the creation of a chimeric Bcr-Abl gene which appears to play a central role in leukemogenesis. Induction of clonal expansion associated with Bcr-Abl expression may be due in part to increased tyrosine kinase activity. In addition, CD34⁺ bone marrow cells from patients with CML respond to colony stimulating factors, but their adhesion to the stroma is impaired, resulting in a loss of sensitivity to stromal inhibitory signals. Currently, allogeneic bone marrow transplantation is the only curative therapy for CML patients, but it is applicable only in relatively young patients with HLA-identical donors. Treatment of CML with recombinant INFα had been the “standard of care” for the treatment of CML for over a decade, resulting in frequent hematological and a lower rate of major or complete cytogenetic remissions in newly diagnosed patients with CML. (reviewed in Strander, 1986). Bcr-Abl RT-PCR negativity was only observed in very few patients. (Guo et al., 2002; Kantaijian et al., 2003). In a recent study, the combination of IFNα and Ara-C induced complete hematological remissions (CHR) in 69% of patients with none achieving PCR negativity (S. O'Brien, personal communication).

Recently, a targeted kinase inhibitor (STI571, Imatinib, Gleevec), was introduced for the therapy of CML. Tyrosine kinase activity of Bcr-Abl is required for the transformation of hematopoietic cells, and ST1571 (for specific tyrosine kinase inhibitor) inhibits Bcr-Abl, Tel/Abl, and V-Abl kinase activity and inhibits growth and viability of cells transformed by any of these ABL oncogenes. (Kantaijian et al., 1995). STI571 can cure mice injected with human leukemic cells, but treatment fails in animals that have large tumors when treatment is initiated (Broxmeyer et al., 1983). Importantly, ST1571 has induced high hematological remission (>90%) and low relapse rates in patients with chronic phase CML. Complete cytogenetic remissions were observed in 95% of patients, but RT-PCR negativity was achieved in only 8% (S. O'Brien, personal communication). ST1571 is highly active in CML patients resistant to IFNα suggesting lack of cross-resistance, but has only limited activity in CML undergoing blastic transformation of CML or in Ph′ positive acute lymphocytic leukemia (ALL). A number of reports detailing STI mediated drug resistance mechanisms have been published. (Blagosklonny 2002). Amplification of the Bcr-Abl gene, an increase in p210 Bcr-Abl protein, and defective AKT/STAT5 signaling have been identified as potential mechanisms of STI resistance. Most notably, mutations of Bcr-Abl have been associated with STI resistance. The observed resistance to ST1571 and low frequency of PCR negativity suggests that CML cells may develop STI-resistance and that alternative approaches may still be required for curing the disease. Numerous investigations are ongoing to test combinations of STI571 with other agents.

Prior to the use of ST1571, the standard of care for CML was systemic administration of INFα. A major problem with the systemic delivery of INFα is its short half-life in vivo, thereby requiring a large bolus injection of drug to achieve therapeutic effect. This was associated with significant side effects and many patients were unable to tolerate the doses required for maximal effect (5 MU/m2 QD.S.C.). As an alternative to daily administration, longer acting formulations of INFα that contain a polyethylene glycol coating (PEG-INFα) were developed. This coating purportedly allows a once weekly dosing (instead of daily). However, this change in formulation did not have major impact on the response of CML to therapy. The combination of IFNα with low doses of Ara-C was found superior to IFNα alone. In summary, systemic administration of INFα has been effective, but its utility is limited, in part, by the inability of patients to tolerate the large exogenous doses required. In general, higher doses have increased clinical efficacy, but less than 30% of patients who received INFα for CML were able to maintain high concentrations, and while the overall response rate was higher, few patients achieved long-term remissions. Since IFNα has been used in CML for many years now, studies of the clinical significance of complete cytogenetic remission (CCR) were possible. Bacigalupo et al. (2001) reported that CCR's were lost in 42% of CML patients at 5 years, and 50% at 8 years implying that CCR's predicted extended disease-free survival in only half of the patients who achieved it. Similar data for STI571 treated patients are not yet available and it will take years to obtain them. However, the reappearance of PCR positivist following allogeneic BMT for CML is frequently followed by relapse suggesting that the inability of achieving PCR negativity in the majority of CML patients treated with STI571 could translate in high relapse rates.

The mechanism by which INFA exerts its antileukemic effects in responding patients remains poorly understood. A variety of in vitro effects of INF( on CML stroma and cells from CML patients have been reported, including inhibition of CML progenitor growth, restoration of adherence to stroma, regulation of stromal cytokine production, and cellular immune surveillance which has been implicated in the control of growth of the leukemic clone in CML. Interestingly, Dr. Jeffrey Molldrem has recently demonstrated the presence of CML-specific cytotoxic T-lymphocytes (CTL) that recognize the hematopoietic antigen PRI on their leukemic target cells and kill them. A strong correlation was observed between the presence of PR1 specific T-cells and clinical responses after IFNα and allogencic bone marrow transplantations.

An alternative to the systemic delivery of INFα could be the autocrine or paracrine production of this protein through integration and expression of the gene intrinsically, i.e., through the use of implanted donor cells expressing INFα. However, donor cells must fulfill several criteria: (1) the cells must be easily obtained, (2) survive for periods of time ex vivo, (3) efficiently express the transgenic, and (4) not elicit a host immune response. Gene transfer and expression studies using transient or stable expression of INFα in CML mononuclear cells, cord blood, CD34⁺ cells, and fibroblasts have shown that other cell types can express a bioactive lymphocyte, and that the exogenous INFα acts similarly to systemically administered INFα. One report has demonstrated that INFα expressing fibroblasts implanted into the hind flank of a tumor-bearing mouse, resulted in decreased tumorigenicity, and strongly suppressed proliferation of the KU182 CML cell line in vivo. However, the use of fibroblast or hematopoietic cells as donor cells does not fulfill the criteria listed above. In certain embodiments, it is contemplated that the source may be donor cells, bone marrow derived, and mesenchymal stem cells. Marrow stromal cells are multipotent stem cells that form an essential structural and functional component of the bone marrow microenvironment and are critical for hematopoiesis. These cells serve as long lasting precursors for bone marrow, bone, cartilage, and connective tissue, and have been studied extensively. They do not express the hematopoictic antigens CD34 and CD45 and MSC grown from leukemia patients have been found by to be free of clonal cells. MSC can be easily obtained from patient or murine bone marrows, isolated by their adherence to plastics, cultured, expanded and engineered in vitro for prolonged periods, and autologously transplanted into the same patient. Studies have shown that transplantation of MSC from one syngeneic mouse via intravenous routes back into other mice results in trafficking of MSC back to bone marrow sites and contribute to repopulation of irradiated bone marrows. In patients with osteogenesis imperfecta, allogeneic MSC were found in the host marrow at a frequency of 7% for up to 18 months, contributing significantly to bone density and reducing spontaneous fractures. Other studies in primates and humans confirmed the ability of MSC to home back to the marrow, proliferate and survive for extended periods of time.

Significant advances have been made in the generation of vectors that direct efficient long-term expression of transgenes. Recombinant adeno-associated virus (AAV) represents a vehicle for gene delivery and has shown promise for in vivo and ex vivo gene therapy applications. Recombinant AAV does not contain sequences encoding viral proteins and has the potential to integrate into chromosomal DNA. Production and purification procedures are now available that allow the generation of AAV without significant contamination with wild-type AAV or helper adenovirus. Recently, a novel method of vector purification based on ion-exchange chromatographs has been described that is scalable and applicable to AAV serotypes a2 and 5. AAV has been shown capable to infect post mitotic cells, such as neurons, and muscle. Studies conducted on a number of organs and tissues using AAV have demonstrated efficient, stable long-term gene expression (up to 1.5 years) with little AAV associated inflammation of cellular response. Current studies from Wilson et al. have shown tightly-regulated gene expression of human growth hormone (hGH) in a mouse model utilizing an AAV carrying a rapamycin inducible promoter. Their data suggest that 6 hrs after drug administration, hGH levels become detectable and maintain high level expression for as long as the drug is present. Clearance of rapamycin results in an immediate decrease in hGH levels. Additionally, readministration of rapamycin results in a rapid resynthesis of hGH and a corresponding increase in expression levels, and this cycle can be repeated daily, weekly or monthly for up to 300 days post one-time AAV administration.

Delivery of therapeutic proteins by gene therapy has the potential to improve the efficacy, convenience, and cost-effectiveness of treatment of a variety of diseases by allowing frequent injection of expensive recombinant proteins to be replaced by the infrequent, or one-time, delivery of therapeutic genes. The spectrum of diseases that can be treated may also be expanded by allowing delivery of proteins that cannot be administered effectively by injection because of poor pharmacokinetics, narrow therapeutic windows, or systemic side effects. In certain embodiments, a recombinant AAV may be constructed that expresses INFα under the control of a drug-regulated promoter. This construct will be utilized to infect MSC. After expansion, these MSC will be transplanted into animals that contain established CML. The use of INFα-expressing MSC will alleviate the need for long-term daily systemic injections of INFα, and will allow drug regulated high-level expression in a localized site-specific manner, thereby reducing systemic side effects.

### B. Melanoma

Cutaneous melanoma is increasing worldwide at a rate exceeding that of all other solid tumors except lung cancer in women. Chemotherapy is minimally effective in recurrent melanoma. Unique amongst solid tumors is its sensitivity to immune-modulated therapies, such as INF-α. Basal cell carcinoma (BCC) and squamous cell carcinoma (SCC) (known collectively as nonmelanoma skin cancer) and malignant melanoma are the most common cutaneous malignancies. Treatment has 3 goals: complete eradication of the cancer and preservation or restoration of normal function. Risk of recurrence or metastasis determines whether the tumor is high risk or low risk. Choice of treatment approach depends on the tumor's location, size, borders, and growth rate. The standard treatment approaches are superficial ablative techniques (electro-desiccation and curettage and cryotherapy) used primarily for low-risk tumors and full-thickness techniques (Mohs micrographic surgery, excisional surgery, and radiotherapy) used to treat high-risk tumors.

As the nature of immune response to melanoma becomes further characterized, it is likely that more specific immune manipulations may be approached clinically. The fact that complete and partial remissions are induced in some patients with metastatic malignant melanoma by INF-α, IL-2, LAK cells, TIL cells, tumor vaccines, and the like clearly indicates a potential role for these agents in the treatment of melanoma. As the overall response rates to these maneuvers are only in the range of 20%, improved treatment methods are needed.

INF-α has a documented activity against metastatic melanoma. The role of immune mechanisms in the control of malignant melanoma and other cancers is suggested by several studies. IFN-α treatment has been shown to result in recruitment of CD4⁺ cells to the proximity of tumor cells. In certain embodiments, methods of treating melanoma with the genetically modified cells of the present invention are contemplated. The localization of genetically modified MSC or precursors that are capable of forming or associating with the stromal components of proliferating or hyperproliferating cells may produce a therapeutic agent, such as INF-α, locally and at higher local concentrations to the stimulate, increase, or enhance a local biological response with in a tumor or cancer locality. In particular embodiments, the cells of the present invention may express INF-α or INF-β.

### VI. Pharmaceutical Preparations

Pharmaceutical compositions of the present invention comprise an effective amount of one or more genetically modified cells or additional agents dissolved or dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. The preparation of an pharmaceutical composition that contains at least one genetically modified cell or additional active ingredient will be known to those of skill in the art in light of the present disclosure, as exemplified by Remington's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the therapeutic or pharmaceutical compositions is contemplated.

The genetically modified cell(s) may comprise different types of carriers depending on whether it is to be administered in solid, liquid or aerosol form, and whether it need to be sterile for such routes of administration as injection. The present invention can be administered intravenously, intradermally, intraarterially, intraperitoneally, intralesionally, intracranially, intraarticularly, intraprostaticaly, intrapleurally, intratracheally, intranasally, intravitreally, intravaginally, intrarectally, topically, intratumorally, intramuscularly, intraperitoneally, subcutaneously, subconjunctival, intravesicularlly, mucosally, intrapericardially, intraumbilically, intraocularally, orally, topically, locally, inhalation (e.g., aerosol inhalation), injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art (see, for example, Remington's Pharmaceutical Sciences, 18th Ed. Mack Printing Company, 1990, incorporated herein by reference).

The actual dosage amount of a composition of the present invention administered to an animal or patient can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of an active compound. In other embodiments, the an active compound may comprise between about 2% to about 75% of the weight of the unit, or between about 25% to about 60%, for example, and any range derivable therein. In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

In any case, the composition may comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

The genetically modified cell(s) may be formulated into a composition in a free base, neutral or salt form. Pharmaceutically acceptable salts, include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine.

In embodiments where the composition is in a liquid form, a carrier can be a solvent or dispersion medium comprising but not limited to, water, ethanol, polyol (e.g., glycerol, propylene glycol, liquid polyethylene glycol, etc), lipids (e.g., triglycerides, vegetable oils, liposomes) and combinations thereof. The proper fluidity can be maintained, for example, by the use of a coating, such as lecithin; by the maintenance of the required particle size by dispersion in carriers such as, for example liquid polyol or lipids; by the use of surfactants such as, for example hydroxypropylcellulose; or combinations thereof such methods. In many cases, it will be preferable to include isotonic agents, such as, for example, sugars, sodium chloride or combinations thereof.

In other embodiments, one may use eye drops, nasal solutions or sprays, aerosols or inhalants in the present invention. Such compositions are generally designed to be compatible with the target tissue type. In a non-limiting example, nasal solutions are usually aqueous solutions designed to be administered to the nasal passages in drops or sprays. Nasal solutions are prepared so that they are similar in many respects to nasal secretions, so that normal ciliary action is maintained. Thus, in preferred embodiments the aqueous nasal solutions usually are isotonic or slightly buffered to maintain a pH of about 5.5 to about 6.5. In addition, antimicrobial preservatives, similar to those used in ophthalmic preparations, drugs, or appropriate drug stabilizers, if required, may be included in the formulation. For example, various commercial nasal preparations are known and include drugs such as antibiotics or antihistamines.

In certain embodiments the genetically modified cell(s) is prepared for administration by such routes as oral ingestion. In these embodiments, the solid composition may comprise, for example, solutions, suspensions, emulsions, tablets, pills, capsules (e.g., hard or soft shelled gelatin capsules), sustained release formulations, buccal compositions, troches, elixirs, suspensions, syrups, wafers, or combinations thereof. Oral compositions may be incorporated directly with the food of the diet. Preferred carriers for oral administration comprise inert diluents, assimilable edible carriers or combinations thereof. In other aspects of the invention, the oral composition may be prepared as a syrup or elixir. A syrup or elixir, and may comprise, for example, at least one active agent, a sweetening agent, a preservative, a flavoring agent, a dye, a preservative, or combinations thereof.

In certain preferred embodiments an oral composition may comprise one or more binders, excipients, disintegration agents, lubricants, flavoring agents, and combinations thereof. In certain embodiments, a composition may comprise one or more of the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc.; or combinations thereof the foregoing. When the dosage unit form is a capsule, it may contain, in addition to materials of the above type, carriers such as a liquid carrier. Various other materials may be present as coatings or to otherwise modify the physical form of the dosage unit. For instance, tablets, pills, or capsules may be coated with shellac, sugar or both.

Additional formulations which are suitable for other modes of administration include suppositories. Suppositories are solid dosage forms of various weights and shapes, usually medicated, for insertion into the rectum, vagina or urethra. After insertion, suppositories soften, melt or dissolve in the cavity fluids. In general, for suppositories, traditional carriers may include, for example, polyalkylene glycols, triglycerides or combinations thereof. In certain embodiments, suppositories may be formed from mixtures containing, for example, the active ingredient in the range of about 0.5% to about 10%, and preferably about 1% to about 2%.

Sterile injectable solutions are prepared by incorporating the genetically modified cells and/or active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and/or the other ingredients. In the case of sterile powders for the preparation of sterile injectable solutions, suspensions or emulsion, the preferred methods of preparation are vacuum-drying or freeze-drying techniques which yield a powder of the active ingredient plus any additional desired ingredient from a previously sterile-filtered liquid medium thereof. The liquid medium should be suitably buffered if necessary and the liquid diluent first rendered isotonic prior to injection with sufficient saline or glucose. The preparation of highly concentrated compositions for direct injection is also contemplated, where the use of DMSO as solvent is envisioned to result in extremely rapid penetration, delivering high concentrations of the active agents to a small area.

The composition must be stable under the conditions of manufacture and storage, and preserved against the contaminating action of microorganisms, such as bacteria and fungi. It will be appreciated that endotoxin contamination should be kept minimally at a safe level, for example, less that 0.5 ng/mg protein.

In particular embodiments, prolonged absorption of an injectable composition can be brought about by the use in the compositions of agents delaying absorption, such as, for example, aluminum monostearate, gelatin or combinations thereof.

### VII. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Material and Methods

### Cells Isolation and Culture

Human MSC were isolated from the bone marrow of normal individuals undergoing bone marrow harvest for allogeneic bone marrow transplantation following informed consent according to institutional guidelines under the approved protocol. Mononuclear cells were separated by centrifugation over a Ficoll-Hypaque gradient (Sigma, St. Louis, Mo.) and suspended in α-MEM medium containing 20% fetal bovine serum (Gibco BRL, Rockville, Md.), L-glutamine, and penicillin-streptomycin mixture (Flow Laboratories, Rockville, Md.) followed by plating at an initial seeding density of 1×10⁶ cells/cm². After 3 days, the non-adherent cells were removed by washing with PBS and monolayers of adherent cells were cultured until they reached confluence. Cells were then trypsinized (0.25% trypsin with 0.1% EDTA), subcultured at densities of 5,000-6,000 cells/cm² and used for studies during passages 3 to 4.

The A375SM and MDA 231 cell lines were a gift from Dr. J. Fidler (Department of Cancer Biology, M.D. Anderson Cancer Centrum, Houston, Tex.). Cells were maintained in α-MEM with 10% FCS, sodium pyruvate, non-essential amino acids, L-glutamine, vitamin solution (Life Technologies, Inc., Grand Island, N.Y.), and penicillin-streptomycin mixture.

### Adenoviral Vectors and MSC Transduction

Adenovirus (AdV) was created using the bacterial plasmid recombination system Ad Easy (Qbiogene). Briefly, the gene for β-galactosidase (β-gal) was cloned into the Not1/HindIII digested Ad CMV shuttle. The gene for human IFN-β was purchased from InvivoGen (San Diego, Calif.), digested with CLAI, and filled in to achieve a blunt end. This blunt ended plasmid was further digested with BglII to release the 570 bp fragment containing hIFN-β, and this piece was subcloned into the BglII/EcoRV sites of pShuttle CMV. These two clones were sequenced to determine the correct reading frame and any possible mutations. The two plasmids were linearized with PmeI, dephosphorylated using calf-alkaline phosphatase, extracted with two rounds of phenol chloroform, and mixed with PacI digested pAdEASY-1. These two linearized plasmids were electroporated into bacteria; plated on Kan+ agar and kanamycin resistant clones were picked and analyzed for AdEASY sequences. We identified 4 clones of each gene (β-gal, IFN-β) and these plasmids were expanded in a 3 ml miniprep format and transfected into 293 cells using Fugene6. After 18-20 days, plaques were eluted and recombinant virus rescued from the cultures. The inventors performed two rounds of amplification, and virus expressing IFN-β as identified by ELISA (Fujirebio Inc, Tokyo, Japan), or expressing β-gal (as detected by histochemical staining) was chosen. MSC were incubated with AdV at MOI=3000 for 2 h. MSC produced 3-4×10⁴ IU of IFN-β per 10⁶ MSC during the first 24 h after infection. β-gal expression in MSC was determined by histochemical stain and more than 90% of MSC were positive.

### In Vitro Antiproliferative Assays

Cell monolayers were washed with PBS, harvested with trypsin, and resuspended in RPMI 1640 with 10% FCS. Cells were plated in 200 μl of media at 3000 cells per well into 96 well plates. Cells were allowed to adhere to the plate overnight, then media with IFN-β was added in different dilutions (range from 0-10,000 IU/ml). Eight wells were used for each dilution. One plate was read by MTS assay (Promega Inc, Madison, Wis.) at the time of initial addition of IFN-β, to serve as initial control. Media with IFN-β (Avonex, Biogen, Inc.) was changed daily, and after five days, the assay was read using MTS. Absorbance was measured at 490 nm. Results were calculated as: % growth=(ODₑₓₚ-ODᵢₙᵢ)/(OD_{fin}-ODᵢₙᵢ)×100. OD_{fin} corresponds to A₄₉₀ of wells with no treatment, ODᵢₙᵢ corresponds to initial control, and ODₑₓₚ corresponds to wells treated with different concentrations of IFN for 5 days.

### Coculture of MDA 231 and A375SM Melanoma Cells with MSC in Vitro

A375SM melanoma cells (5×10⁴ per well) or MDA 231 breast cancer cells (10⁵ per well) were cultured either alone or mixed with MSC and IFN-β-MSC, respectively at a ratio 10:1 in six-well plates. After 5 days, cells were trypsinized, counted and fixed with 70% ethanol. Then, cells were labeled with PE (Sigma) and cell DNA content analyzed using the FACScan flow cytometer (Becton-Dickinson, San Jose, Calif.). The relative numbers of MSC (diploid cells) and A375 or MDA 231 cells (aneuploid cells) were determined using ModFit software (Verity Software House, Inc., Maine).

### Co-Culture of Ovar-3, SKOV-3, and Hey Cells with MSCs in Vitro

Mesenchymal stem cells were infected with an adenovirus carrying the IFN-β gene (MSC-IFNβ), to produce levels of 40,000 IU/5×10⁵ cells/ 24 hours. Another flask of MSCs was infected with an adenovirus carrying the beta-galactoside gene (MSC-βGAL), at levels to achieve 95-100% transfected cells. After 24 hours, cell monolayers were washed with PBS and removed using trypsin-EDTA, all cell lines were then resuspended in RPMI 1640 with 10% FBS. OVAR-3, SKOV-3, or Hey cells were plated in 4 ml of medium either alone or mixed with MSC-IFNβ or MSC-βgal in a ratio of 1:1 or 10:1 respectively in six-well plates at a starting concentration of 4×10⁴ cells per well. After 5 days, cells were trypsinized, counted, and fixed with 70% ethanol. Cells were then labeled with PE (Sigma), and the cell DNA content was analyzed using the FACScan flow cytometer (Becton-Dickinson, San Jose, Calif.). The relative numbers of MSCs (diploid cells) and ovarian carcinoma cells (aneuploid cells) were determined using ModFit software (Verity Software House Inc, ME).

### Animals, Cells Administration, Tumors and Survival Analysis

Female C.B-17 SCID mice were purchased from Harlan (Indianapolis, Ind.). Mice were used in accordance with institutional guidelines under the approved protocols. Cells were administered suspended in 200 μl of PBS intravenously into the lateral tail vein. Tumor burden was determined by measuring the weight of whole lungs. The difference in lung weight was determined by two-tail t test. Survival was measured from the day of MDA 231 or A375SM cells injection until day of death. Difference in survival was determined by two-tail log rank test. Statistical analysis was performed using Statistica software (StatSoft, Inc., Tulsa, Okla.).

### Tissue Processing and Imaging Studies

Lungs and other organs were fixed in Bouin's solution or embedded in OTC compound (Miles, Inc., Elkhart, Ind.), then snap frozen in liquid nitrogen and stored at −80° C. Additionally, whole lungs of several animals were immediately stained for β-Galactosidase by X-Gal staining. Frozen tissue was sectioned (6-8 μm) and processed for H&E or X-Gal histochemical staining. Imaging was performed with Zeiss Axioplan2 microscope (Carl Zeiss, Inc., Thornwood, N.Y.) equipped with a CCD camera (Hamamatsu Corp., Bridgewater, N.J.) and processed using Adobe Photoshop software (Adobe Systems, Inc., San Jose, Calif.).

### X-Gal Histochemical Stain

Whole lungs were fixed in 0.5% glutaraldehyde for 10 min and washed with PBS. Tissues was then incubated with 2% X-Gal solution (Sigma) with 1M MgCl₂, 30 mM potassium ferricyanide and 30 mM potassium ferrocyanide overnight and refixed in 10% neutral buffered formalin. Tissues were dehydrated with ethanol and after minimal exposure to xylene, embedded in paraffin and cut into 5 μm slides. Then, slides were deparafinized and couterstained with eosin or Nuclear Fast red. Alternatively, slides from frozen tissues were fixed with cold aceton/ethanol 1:1 for 20 min. and stained with X-Gal.

### Measurement of IFN-β Concentration in Mouse Plasma

Mice with established MDA 231 metastasis in lungs were injected with 10⁶ MSC-IFN-β intravenously or subcutaneously. Other animals received 40,000 IU or 100,000 IU of IFN-β (Avonex, Biogen, Inc.) subcutaneously. 200 μl of blood was collected into heparinized capillaries at appropriate intervals from cuts of the tail vein. Blood was immediately centrifuged to remove cells and plasma stored at −80° C. Concentration of IFN-β in plasma was determined by ELISA (Fujirebio Inc, Tokyo, Japan) using the NIH standard of IFN-β1a.

### MSC Labeling with the Fluorescent Dye SP-Dil

The fluorescent dye SP-DiI (Molecular Probes, Eugene, Oreg.) was dissolved in dimethylformamide (Sigma) to the concentration of 2.5 mg/ml. SP-DiI dye was then added directly to culture medium to a final concentration of 10 μg/ml. MSCs (4×10⁶ cells) were incubated with 25 ml of medium with SP-DiI in T175 flask for 48 h. Then, cells were washed with PBS, incubated with dye-free medium for 4 h and used for studies.

### Tumor Measurements and Determination of Animal Survival

Tumors were measured by caliper, and tumor area was calculated as the geometric mean of two perpendicular diameters. Survival was measured from the day of cell injection to death, or when the mouse had to be sacrificed secondary to tumor diameter>15 mm, tumor ulceration, or bleeding. The difference in survival was determined by log rank test.

### Imunohistochemistry with AS02 Antibody

Slides were fixed in cold acetone, and endogenous peroxidase was blocked by 3% hydrogen peroxide in methanol. Nonspecific binding was blocked by incubation with F(ab₂) IgG fragment of goat antimouse antibody (Jackson, West Grove, Pa.; dilution 1:10), 5% horse serum, and 1% goat serum in PBS for 24 h at 4° C. Primary mouse antihuman AS 02 antibody (Dianova, Inc., Hamburg, Germany; dilution 1:20) was used overnight at 4° C., followed by peroxidase-conjugated rat antimouse IgG1 antibody (PharMingen, San Diego, Calif.; dilution 1:600) for 1 h at room temperature. Positive reaction was visualized with stable 3,3′-diaminobenzidine (Research Genetics, Huntsville, Ala.).

### Immunofluorescence Staining for BrdUrd

Two hundred μI of 10 mM BrdUrd (Sigma) dissolved in PBS was administered i.v. 4 and 2 h before animals were sacrificed. Slides were fixed with 4% paraformaldehyde, treated with 0.1% Triton X-100 in PBS, incubated with 2 N HCl for 30 min at 37° C., and washed with 0.1 M Tris. Then, slides were incubated with primary mouse anti-BrdUrd antibody (Becton Dickinson, Mountain View, Calif.; dilution 1:100) overnight at 4° C. followed by secondary goat antimouse Alexa 488 antibody (Molecular Probes, Eugene, Oreg.; dilution 1:400) for 1 h at room temperature, and mounted in mounting medium (Vector).

### Example 2

### Interaction of Human Bone Marrow Derived MSCs with A375SM Melanoma Cells

In the following exemplary studies of the interaction of human bone marrow-derived MSC with A375SM melanoma cells (Kozlowski et al., 1984) of human origin in a mouse xenograft model are described. Msc were isolated from normal individuals undergoing bone marrow harvest for allogenic bone marrow transplantation under approval of a protocol according to a method of Pittenger (Pittenger et al., 1999, incorporated herein by reference). MSC were labeled with the fluorescent dye SP-DiI14, pre-mixed with A375SM melanoma cells, and injected subcutaneously into nude mice.

Male athymic nude mice (NCr-nu) were purchased from the Animal Production Area of the National Cancer Institute—Frederick Cancer Research and Development Center (Frederick, Md.). Tumors (n=10) were then examined by fluorescence microscopy and immunohistochemistry with an antibody specific for human fibroblasts that does not cross-react with mouse tissue or human melanoma cells as described in Saalbach et al., 1996. Slides were fixed in acetone, and endogenous peroxidase was blocked by 3% hydrogen peroxide in methanol. Nonspecific binding was blocked by incubation with F(ab₂) IgG fragment of goat anti-mouse antibody (Jackson, West Grove, Pa.) dilution 1:10, 5% horse serum and 1% goat serum in PBS for 24 h at 4° C. Primary mouse anti-human AS 02 antibody (Dianova Inc., Germany), dilution 1:20 was used overnight at 4° C., followed by peroxidase-conjugated rat anti-mouse IgG1 antibody (Pharmingen, San Diego, Calif.) dilution 1:600 for 1 h. A positive reaction was visualized with stable DAB (Research Genetics, Huntsville, Ala.).

These studies revealed that a significant portion of MSC derived fibroblasts were incorporated into the tumor architecture and formed fibrous capsule at the tumor periphery. Many of the MSC-derived fibroblasts in the tumor capsule lost fluorescent dye. Since the SP-DiI fluorescent dye is tightly bound to the cellular membrane and is not transferred to neighboring cells in vivo (Johansson et al., 1999), the fluorescence intensity of labeled MSC declines only during cell division when the membrane of the parental cell is evenly distributed between both daughter cells. Repeated cell divisions lead to a further decrease of the fluorescence signal until it is indistinguishable from the background of the surrounding unlabeled cells. The observed loss in fluorescence intensity of the MSC-derived cells in the tumor capsule may be related to their proliferation and cell division.

Direct evidence of MSC proliferation in tumors was obtained from in vivo BrdU labeling. Briefly, in vivo BrdU labeling methods include: 200 μl of 10 mM BrDU (Sigma, St. Louis, Mo.) dissolved in PBS was administered intravenously 4 and 2 h before animals were sacrificed. Slides were fixed with 4% paraformaldehyde, treated with 0.1% Triton X-100 in PBS, incubated with 2N HCl for 30 min at 37° C., and washed with 0.1M Tris. Slides were then incubated with primary mouse anti-BrDU antibody (Becton Dickinson, Mountain View, Calif.) dilution 1:100 overnight at 4° C., followed by secondary goat anti-mouse Alexa 488 antibody (Molecular Probes Eugene, Oreg.) dilution 1:400 for 1 h. Mice with tumors derived from mixtures of melanoma cells and SP-DiI labeled MSC were intravenously injected with BrdU. Proliferating cells were identified by BrdU immunofluorescence. This method clearly showed SP-DiI labeled MSC with BrdU positive nuclei in tumors. In contrast, BrdU was not incorporated into MSC injected subcutaneously alone without A375SM melanoma cells. The results indicate that bone marrow MSC contribute to tumor stroma formation when co-injected with the malignant cells. This process involves not only passive incorporation of MSC into the tumor architecture, but also their proliferation.

### Example 3

### MSCs Contribute to Tumor Stroma Formation After Intravenous Administration

Further exemplary studies addressed whether MSC contribute to tumor stroma formation after intravenous administration. Mice with established A375SM melanomas growing in the lungs were injected with MSC through the tail vein and then sacrificed after 1, 8, and 60 days. The distribution of MSC-derived cells in melanoma nodules and lung parenchyma was then examined by immunohistochemistry. MSCs were randomly distributed in lung parenchyma and tumor nodules 1 day after their intravenous administration. However, after 8 days, MSCs were found mainly in tumors and had cleared from normal lungs. Similarly, MSCs were detected in tumors but not in lung parenchyma 60 days after injection. The preferential distribution of MSC in tumors but not lungs at the latter time points indicate that tumor microenvironment but not normal lung parenchyma supports their survival and incorporation into stroma. Furthermore, the percentage of MSCs in tumors was approximately stable during the study. Five positive tumors at each time point were evaluated. Cells were counted in 5 fields (×100) from tumor areas that were visually judged to express the highest number of positive cells. Results were expressed as mean±sem. (day 1: 3±2%, day 8: 11±2%, day 60: 5±1%). Because, the tumors size increased between day 1 and 60, the absolute number of MSC-derived cells in individual tumor nodules should also have increased during this time, presumably by proliferation.

### Example 4

### Intravenously Injected MSCs Integrate into Subcutaneous Tumors

Other exemplary studies determined whether intravenously injected MSCs can integrate into subcutaneous tumors. Mice with A375SM melanomas received five doses (10⁶ cells per dose) of unlabeled MSCs over a 20-day period. Mice were sacrificed 15-20 days later and tumors, livers, spleens, and lungs were evaluated by immunohistochemistry. MSC-derived fibroblasts were consistently identified in 55% of tumors, but were not found in other organs except for the rare positive cells seen in the spleens of some animals.

### Example 5

### MSCs as Cellular Vehicles for Production of Anticancer Agents

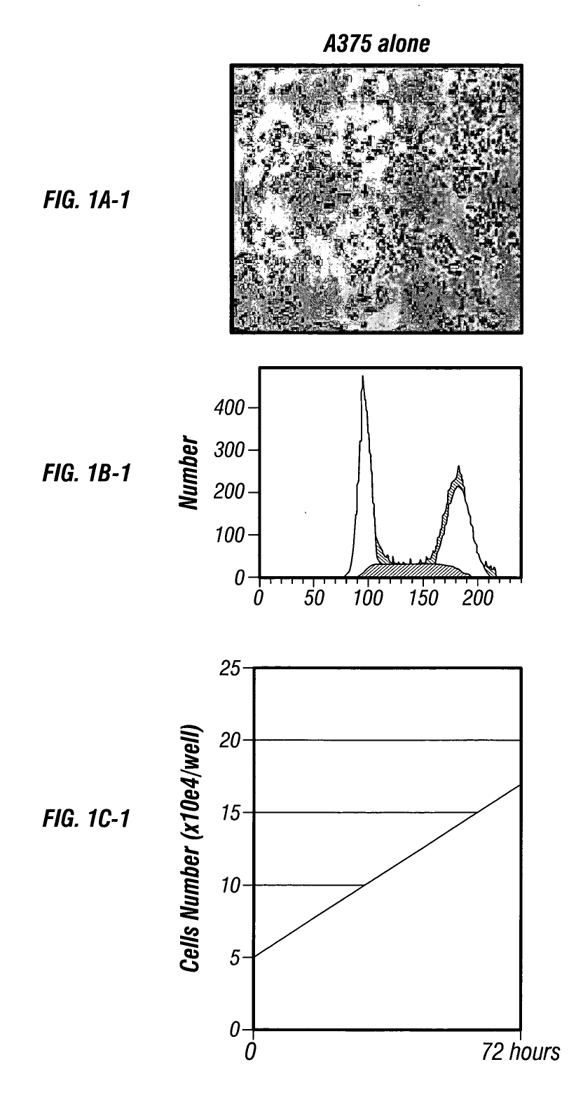

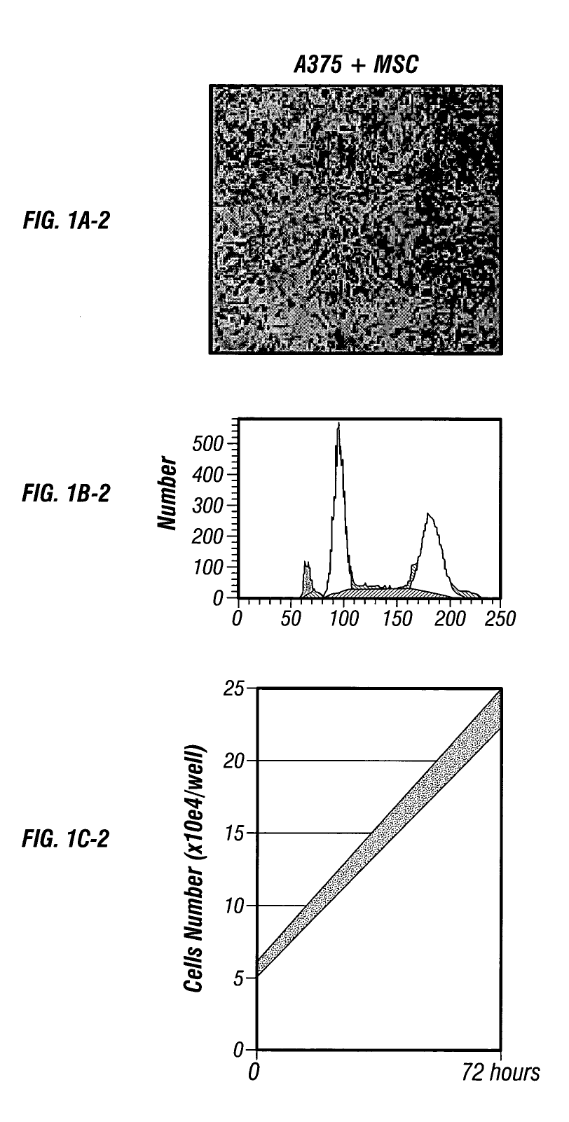

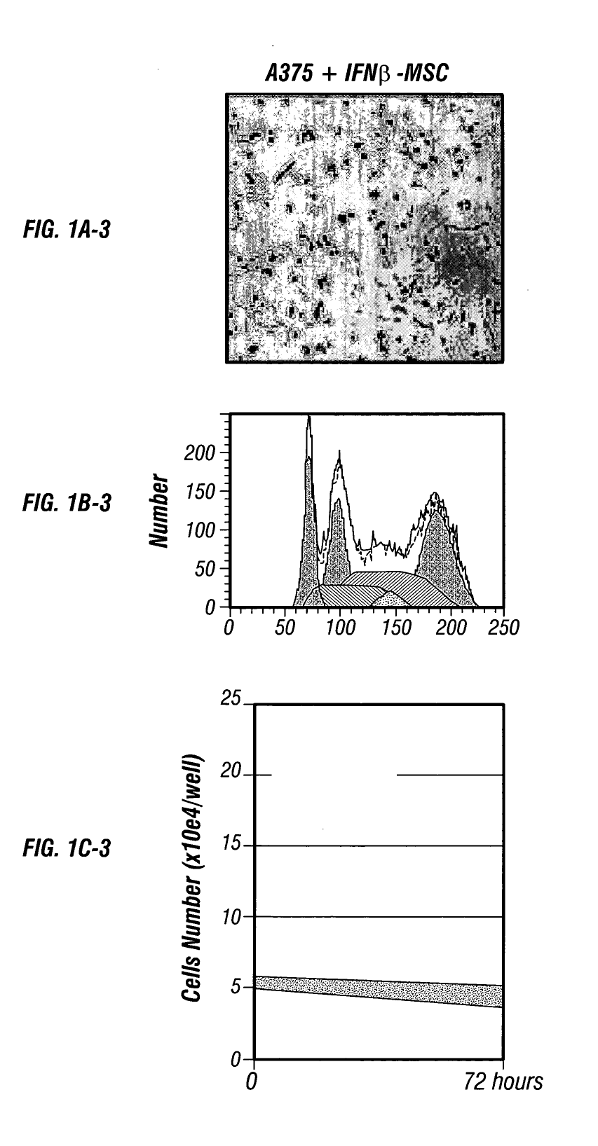

Still other exemplary studies determined the therapeutic potential of MSCs as cellular vehicles for production of anticancer agents after their transduction with an adenoviral vector carrying the human β-interferon (IFN-β) gene. Adenoviruses were created using the bacterial recombination system Ad Easy (Qbiogene, Carlsbad, Calif.). The gene for IFN-β (purchased from Invivogen, San Diego, Calif.) was cloned into the Ad CMV shuttle. The plasmid was linearized with PmeI, mixed with PacI digested pAdEASY-1 and electroporated into bacteria. Selected clones were picked and analyzed for their AdEASY sequences and transfected into 293 cells using Fugene6 after which recombinant virus was rescued from the culture. After two rounds of amplification, virus expressing IFN-β as identified by ELISA (Fujirebio Inc, Tokyo, Japan) was used in subsequent studies. MSC were incubated with adenovirus at an MOI of 50 for 2 h. In vitro, the MSC produced 4-5×10⁴ IU of IFN-β per 10⁶ cells during the first 24 h after transduction. First, the effect of IFN-β producing MSC (IFN-β-MSC) on A375SM melanoma cells in a co-culture system under in vitro conditions was determined, 5×10⁴ IU of Avonex from Biogen was injected subcutaneously every other day. These studies indicated that IFN-β-MSC directly inhibited the growth of malignant cells and did not require the host immune system for this effect FIG. 1.

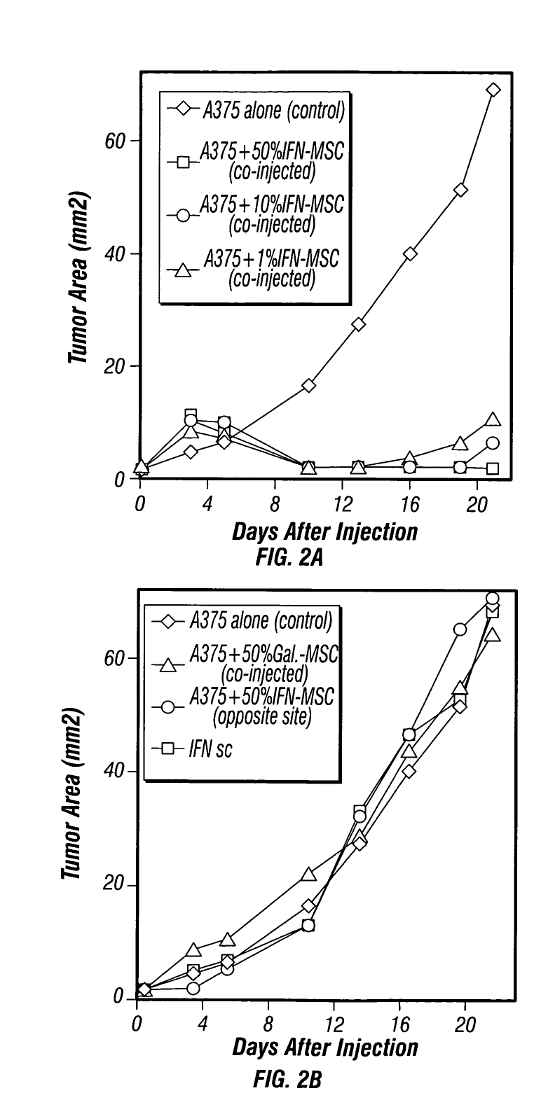

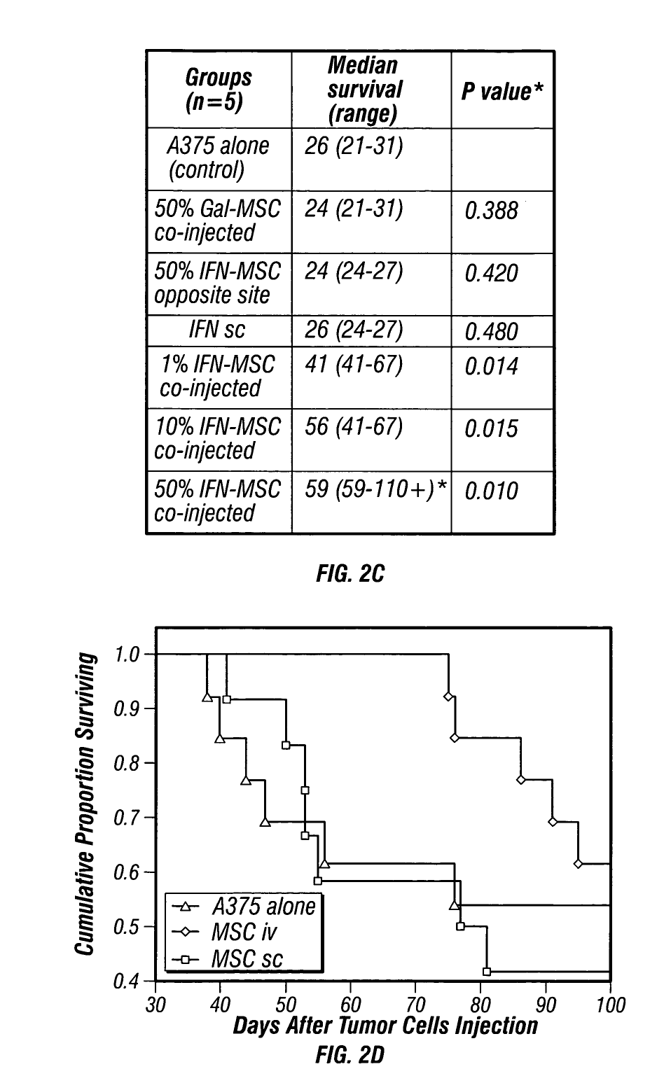

For the in vivo studies, A375SM melanoma cells (10⁶ cells) were co-injected subcutaneously into nude mice together with 5×10⁵, 10⁵ or 10⁴ IFN-β-MSC at the same site. These numbers represented 50%, 10% and 1% of malignant cells and corresponded to the frequency of MSC found in tumors in biodistribution studies (3-11% of all cells in tumors). IFN-β-MSC suppressed tumor growth and prolonged the life of the animals in all of these groups (FIG. 2). A375SM melanoma (5×10⁴ cells) either alone or mixed with MSC and IFN-β-MSC respectively (10⁴ cells) were grown in six-well plates for 72 h.

Cells were then trypsinized and counted. The relative numbers of MSC (diploid cells) and A375 cells (aneuploid cells) were determined using ModFit software (Verity Software House Inc, Me.) after labeling the cells with PE (Sigma) and analyzing DNA content using the FACScan flow cytometer (Becton-Dickinson, San Jose Calif.). Of note, even 1% of IFN-β-MSC (10⁴ cells) were able to exert control of tumor growth and result in significant prolongation of survival (FIG. 2). In contrast, the systemic level of IFN-β supplied by a 50 times higher number of IFN-β-MSC (5×10⁵ cells) injected subcutaneously into the flank contralateral to the site of the tumor or the subcutaneous administration of a corresponding dose of IFN-β did not have any effect on tumor growth or survival. These data indicate that local interferon production in the tumor microenvironment is essential for control of malignant cells and cannot be substituted for by corresponding systemic levels of IFN-β in serum delivered from a distant site.

### Example 6

### Intravenous Administered IFN-β-MSC

In yet another example, a clinically relevant situation was studied to determine the efficacy of intravenously administered IFN-β-MSC in a pre-established metastatic melanoma model. Tumor nodules were allowed to developed in the lungs of mice injected intravenously with A375SM melanoma cells after which the animals received the same number of IFN-β-MSC via one of two different routes. One group received IFN-β-MSC as an intravenous injection through the tail vein and an other group as a subcutaneous injection into the flank. Based on our distribution data, we anticipated that intravenously injected MSC would freely travel via the blood stream, become incorporated into the tumor stroma and produce IFN-β locally in the tumor microenvironment. Conversely, subcutaneously injected IFN-β-MSC do not migrate from the site of injection and produce systemic level of IFN-β. Local production of IFN-β in tumors resulting from intravenously injected IFN-β-MSC did significantly prolong animal survival (p=0.023). In contrast, the systemic levels of IFN-β supplied by the same number of subcuteneously injected IFN-β-MSC had no effect (p=0.21). As expected, the tumor inhibition in the studies was not permanent and corresponded to the relatively short-lived IFN-β expression achievable with conventional adenoviral vectors. However, this hurdle can be overcome by a stable transfection system with regulated protein expression.

Exogenously administered MSCs preferentially survive and proliferate in the presence of malignant cells and become incorporated into the tumor architecture as stromal fibroblasts. This process could be related to high local concentrations of paracrine growth factors such as FGF, PDGF, EGF, TGF-β, or other mediators within the tumor microenvironment (Hanahan and Weinberg, 2000). It has been demonstrated, at least in vitro, that MSC proliferation depends on adequate concentrations of these molecules.

IFN-β has a wide range of biological activities and can induce tumor regression through indirect immunomodulatory (Kuznetsov et al., 1997) and antiangiogenic properties or through direct antiproliferative effects on malignant cells (Le Bon et al., 2001). IFN-β-MSC directly controlled the proliferation of melanoma cells in vitro and do not require the immune system for this effect. Moreover, human IFN-β produced by IFN-β-MSC is species specific and does not directly influence endothelial cells or residual immune cells of mouse origin (Johns et al., 1992). Therefore, the tumor suppression seen in this in vivo model may be related to the direct antiproliferative action of human IFN-β-MSC on human tumor cells.

Clinical studies have shown that the serum concentrations of IFN-β after the systemic administration of the maximally tolerated dose are far below those required to achieve an antiproliferative effects observed in vitro (Qin, et al., 2001). This suggests that direct antiproliferative effect of IFN-β on malignant cells rarely if ever occurs in patients and may explain the disappointing efficacy of this biological agent in clinical trials (Salmon et al., 1996). Embodiments of the invention providing compositions and methods for local production of IFN-β by MSC in the tumor microenvironment can overcome this limitation and simulate the physiological role of IFN-β as a short-range paracrine regulator of cell proliferation and differentiation (Einhom and Grander, 1996). It is also of interest that local deficit in the IFN-β level was detected in tissues surrounding certain tumors which could foster the growth of malignancies (Hertzog et al., 1994; Kuniyasu et al., 2000). Under physiological conditions, IFN-β is produced by cells to influence neighbors spatially located in the same area and, at the same time, avoid interference with regulatory mechanisms that control cells in other parts of the body. Therefore, perhaps, the systemic administration of IFN-β cannot attain this physiological function. The same approaches may be used in the delivery of other agents.

### Example 7

### In Vivo Detection of MSCs

MSCs were harvested from the bone marrows as described in Deans and Moseley, 2000, incorporated herein by reference. MSC have a fibroblast-like morphology, and attach to plastic. Typically 1×10⁷ MSC/10 mls of bone marrow or peripheral blood. MSC were cultured in RPMI with 25% FCS, and require that they be passaged once they reach 80% confluence. These cells can be labeled with membrane binding dyes, such as SP-DIL, and PKH26 (Konopleva et aL., 1999). These dyes allow in vivo monitoring as they fluoresce under UV excitation. The SP-DIL labeled MSC can be injected in nu/nu mice or BalbC/nu mice and detected in cryosections of tissues and organs harvested some time later. SP-Dil labeled MSCs can be detected 30 days after tail vein injection in spleen, lung and bone marrow.

Additionally, MSC can be detected using immunohistochemistry. Briefly, the antihuman AS-02 antibody can recognize cells from mesenchymal origin (Liechty et al., 2000); this allows MSC to be identified in paraffin-fixed samples as well as confirms MSC, which have lost their PKH-26 membrane marking due to cell division in vivo. MSC may be identified by AS-02 staining in mouse tissues 60 days after tail vein injection, whereas PKH-26 labeled cells are present, but not plentiful. MSC engrafling and maintenance in transplanted mice is demonstrated by using gene marked MSC. Briefly, MSC were infected with an AAV-βgal construct and these cells selected to homogeneity using a FACS sorter. These cells were expanded and injected IV in mice. At various times after injection, mice organs and tissues were subjected to PCR™ analysis utilizing Pgal primers as described in Marini et al. (1995), incorporated herein by reference. βgal+ amplimers were detected in tissues harvested from MSC injected mice 7 days, 1, 2, and 3 months post injection. Of note is that certain tissues are negative for βgal amplimers, and MSC which were detected initially (as in brain, kidney) have disappeared suggesting a preferential growth of MSC in the host. Mice injected with MSC lacking the βgal gene are negative for βgal+amplimers.

### Example 8

### Recombinant AAV Expressing IFN-α

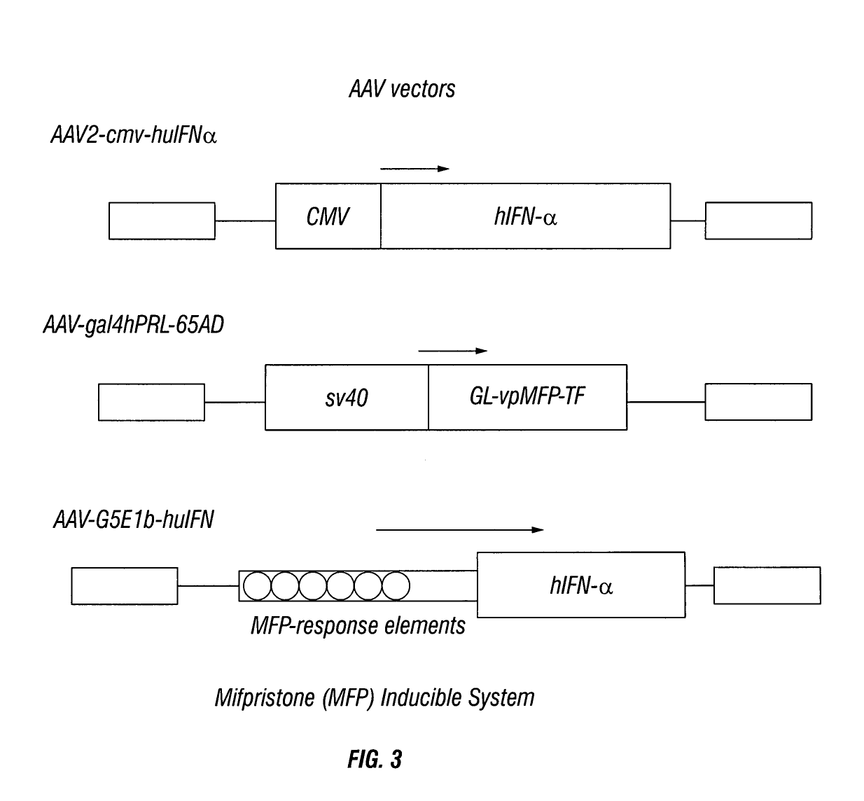

Construction of recombinant AAV expressing IFN-α. MFP1 vectors containing the mifpristone response elements, and transactivating GAL4 protein where cloned into the enhancer site of a mutated CMV enhance/promoter construct. The activation domain consists of a RZF/B fragment fused to the activation domain derived from MFP responsive element (Moraveova et al., 2000). An IRES site allows joint expression of both components from a minimal interleukin-2 promoter. This MFP-regulated promoter was then cloned into an AAV-1 based plasmid, which contains the IFN-α-2B gene (called AAV-gal4hPRL-65AD). As a positive control an AAV was constructed which contained the CMV promoter driving expression of the IFN-α gene (diagram of vectors shown in FIG. 3). The 293 packaging line created by Wilson et al. (1989), contains both REP and CAP functions (PAV1H) in trans. After cotransfection with a helper plasmid (pFD13, with essential regions of the adenovirus genome), the MFP regulated IFN-α-AAV-1 construct were harvest 96 h post transfection, lysed and subjected to 2 rounds of CsCl purification (Cao et al., 2000). Recombinant AAV-IFN was titered on 293 cells and analyzed by DNA hybridization to determine genome equivalent (GE) numbers and size. Vector preparations are provided by Jim Wilson, UPenn Institute of Human Gene Therapy. The ability of AAV to infect MSC in vitro using a CMV-driven GFP vector is illustrated by detection of a strong GFP signal in MSC, and that 10⁵ GE is sufficient to confer GFP expression in greater than 85% of the MSC.

### Example 9

### Inducible AAV Vector

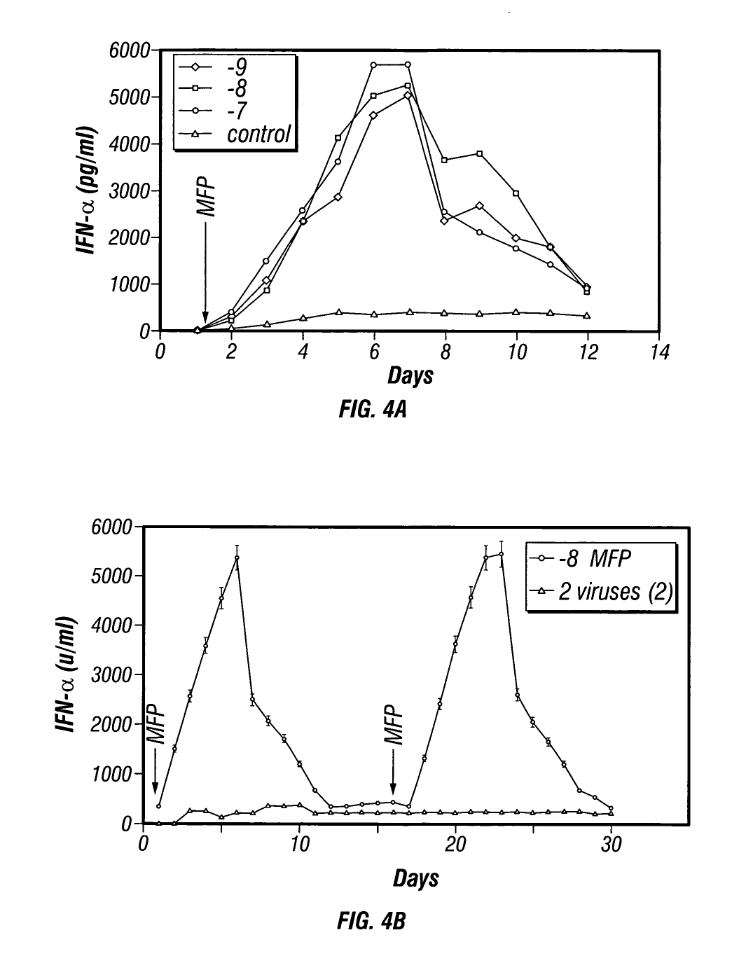

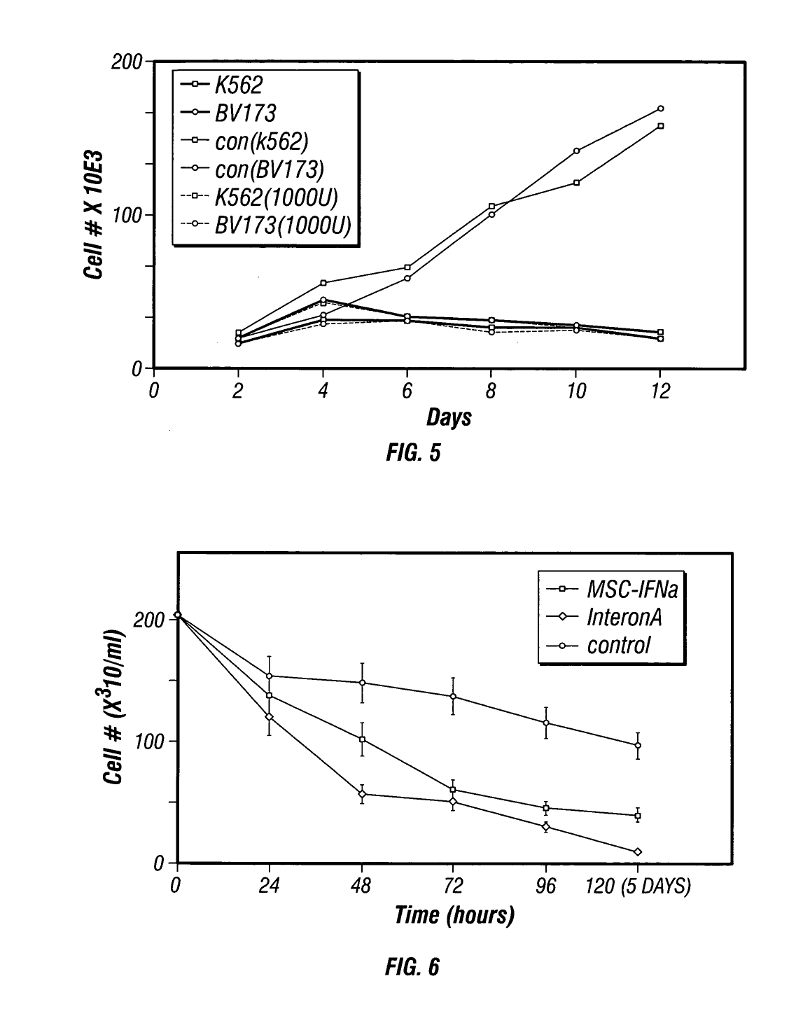

Regulated expression from a drug inducible AAV vector. An AAV construct was used that expressed IFN-α under the control of a MFP-regulated promoter, to infect normal donor MSC in vitro. Briefly, MSC were infected with 10³ GE of AAV-IFN-α and 10³ GE of AAV-gal4prlMFP, and expanded for 10 additional days. These cells were subcultured into 12 well dished were MFP (dissolved in 0.1% ETOH) or solvent carrier was added at the concentrations noted. MSC infected w/ the MFP regulated AAV, express IFN-α only after 18 h exposure to MFP (FIG. 4A). Three drug concentrations were tested, each dose was 10-fold less than the previous one, and with the initial dose of 10⁻⁷ M being 3 logs less than the dose commonly given for chemical abortions in humans. Of note is that a one-time addition of MFP results in a prolonged 8-10 day expression of IFN-α, peaking at 4-5 days after drug addition. This data suggests that for continued expression of IFN-α, MFP may be administered 2 to 3 times weekly. To demonstrate repeat induction of IFN-α expression, this same AAV-IFN-α infected MSC were induced w/10⁻⁸ M MFP. Ten days after IFN-α level reached background activity we waited 3 additional days and added another dose of 10-8 M MFP. IFN-α induction occurs again, and appears to be induced with a similar magnitude and duration of expression as the first time (FIG. 4B).

### Example 10

### Biological Effects of MSC Produced IFN-α on CML Cells

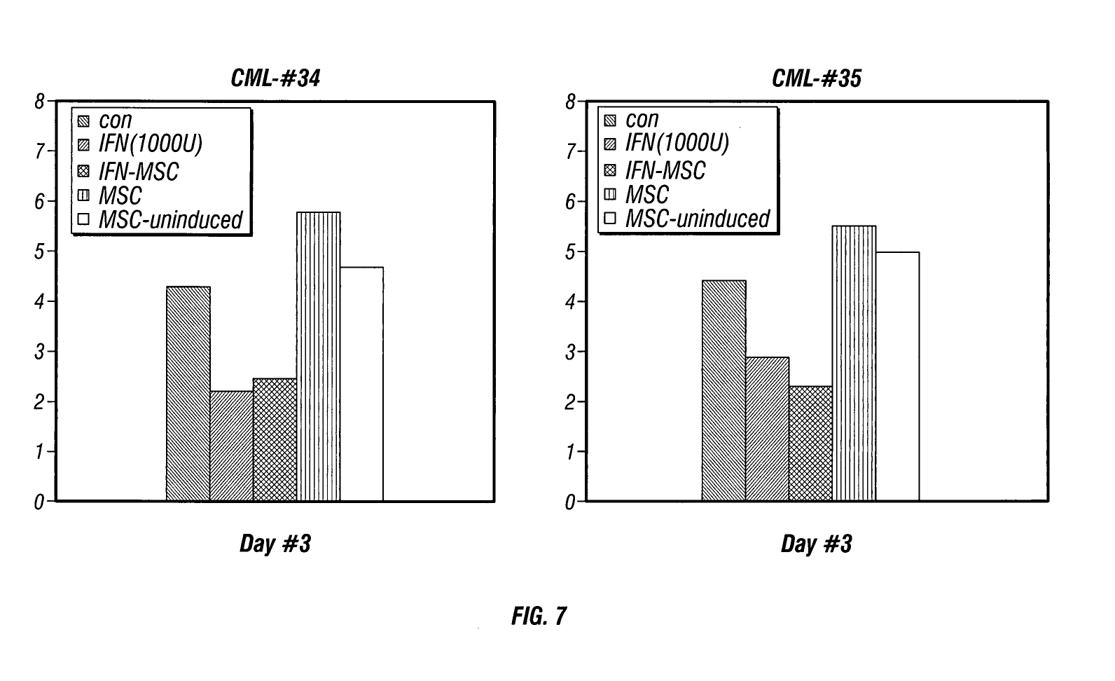

Biological effects of MSC produced IFN-α on CML cell lines and patient samples. The biological activity of this MSC-produced IFN-α on CML (bcr/abl+) cells lines and patient samples was determined. As a positive control through out the studies comparison to pharmacy grade recombinant IFN-α (Intron A, Plough Schering) with IFN-α produced by the MSC were performed. CML cell lines K562 and BV173 (both cell line overexpress bcr/abl Marini et al., 1999) are growth inhibited when cultured with 1000 U of Intron A or cocultured on MSC induced to express IFN-α. Additionally, after 10 days we found very few viable K562 or BV173 cells (as determined by trypan blue) in the coculture set-up. To ensure that the MSC produced IFN-α was also active against BCR-ABL+CML stem cells, CD34 enriched CML stem cells were obtained and cultured in the presence of 1000 U of Intron A or co-cultured on MSC induced to express IFN-α. As shown in FIG. 6 both lymphokines (the Intron A and the MSC-produced IFN-α) were equally effective at reducing growth and viability in CD34+ CML stem cells, suggesting that MSC produced IFN-α is equally effective as Intron A. To further test this hypothesis circulating blasts from 2 CML patients were cultured in medium containing the IFN-α produced by MSC and a culture of Intron A, as shown in FIG. 7 CML blasts cultured in the presence of 1000 U of Intron A or MSC induced to express IFN-α are growth inhibited at a similar rate. Of interest is that if one cultures CML blast cells on a feeder layer of MSC the blast cells grow much better with greater survival and increased proliferation. To ensure that the MSC-produced IFN-α was equally biologically active as Intron A patient samples were examined for upregulation of MHC class I expression. Class I upregulation is considered an important function of IFN-α activity on CML cells as increased cellular immune surveillance has been implicated in the control of growth of the leukemic clone in CML (1e Coutre et al., 2000). Patient samples upregulate Class I when grown in the presence of Intron A or when co-cultured on a MSC feeder layer which has been induced to express IFN-α, or when the supernatant from MSCs-expressing IFN-α is used as culture medium for these patient samples. This data suggests that both MSC-produced IFN-α and Intron A have comparable biological activity.

### Example 11

### In Vivo Testing

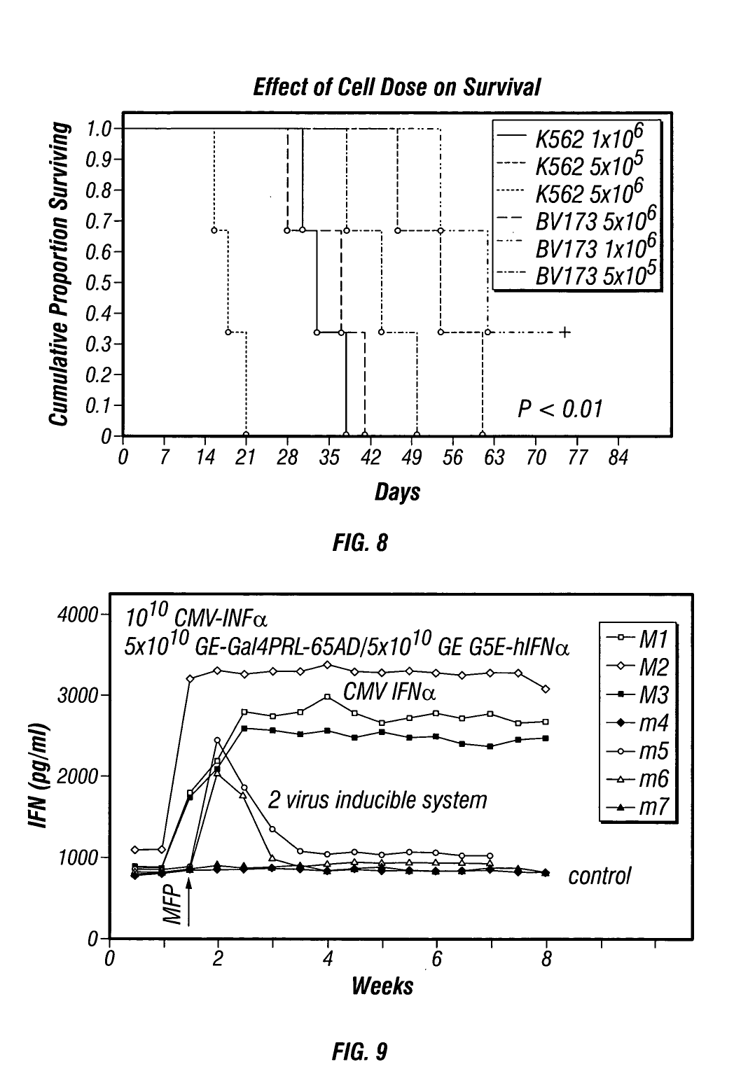

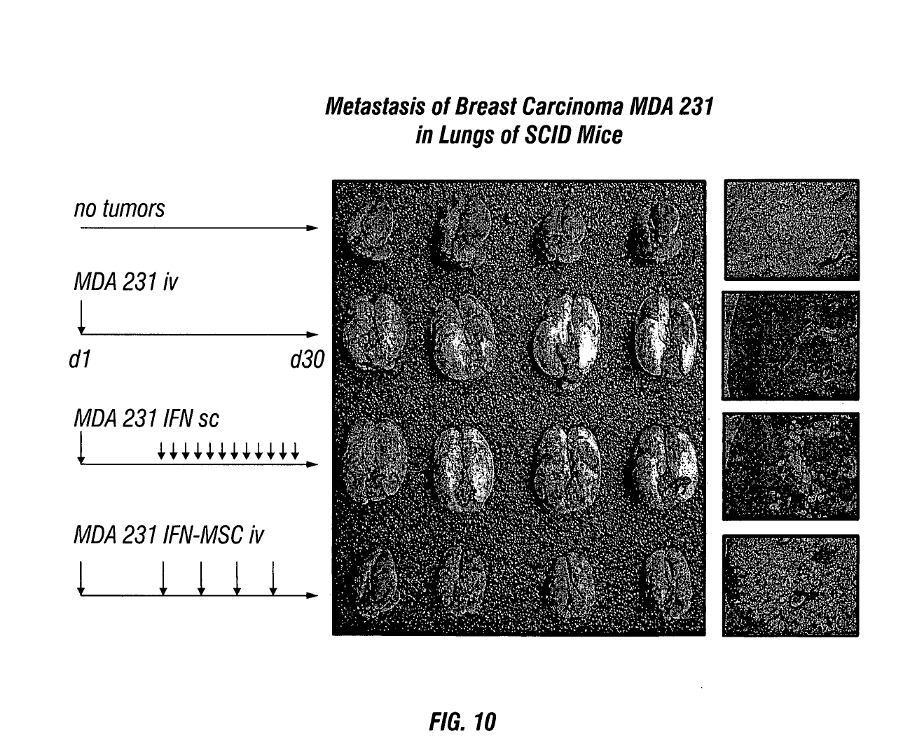

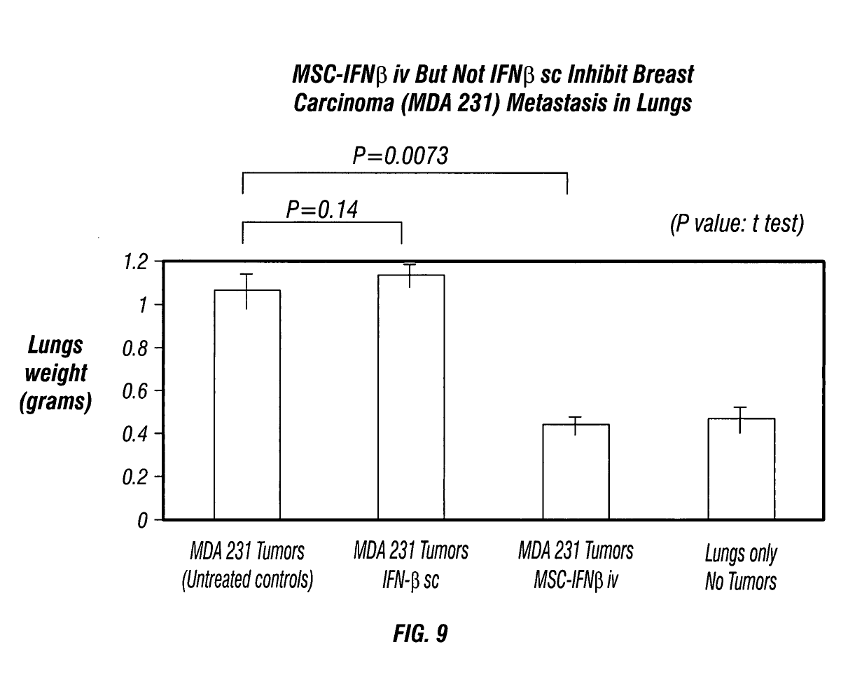

In vivo testing. To initiate a in vivo culture model of CML, two BCR-ABL+ cell lines, the K562 (which grow extramedullary in nu/nu mice) or BV173 (which grows intra bone marrow in BalbC/nu mice) were injected into respective mice. As shown in FIG. 8, a dose of 5×10⁶ K562 cells resulted in the mice dying in approximately 30 days, whereas 1 million K562 cells was lethal within 45 days and half a million K562 injected IV was lethal with in 60 days. Utilizing the BV173 cell line we tested three cell doses and found one million BV173 cells were sufficient to kill the mice within 45 days, whereas 5×10⁵ BV173 cells allowed the mice to survive for 60+ days with one mice still surviving. This data should allow the administration of a lethal cell dose during which cell-based therapies can be administer (the MSC expressing IFN-α). As an alternative to using MSCs as a cell based therapy, the AAV vectors may be directly inject into the muscle, thereby allowing secretion of IFN-α systemically. Evaluation of this route of delivery was performed by an intramuscular injection of 1010 GE AAV-CMV-IFN-α or 5×10¹⁰ GE of AAV-Gal4hPRL-65AD and 5×10¹⁰ GE of AAV-G5E-IFN-α into the quadriceps of mice. Mice were bled weekly and the serum was analyzed for IFN-α expression using the Biosource ELISA kit. As shown in FIG. 9 circulating levels of hIFN-α was detected in the peripheral blood, and more importantly, it was identified over the high background level seen in these nu/nu animals. In three animals injected with the constitutively expressing IFN-α (AAV-CMV-IFN-α) over 2600 pg/ml of hIFN-α were detected, which require 2 weeks to reach maximum levels and this activity has remained constant for the two months surveyed. Additionally, mice injected with the inducible AAVs show a drug dependent induction of IFN-α, mice 6 and 7 have similar induction curves in FIG. 9. Of note is that one application of MFP (given IP) results in a single peak of IFN-α activity which decays over a 7-day period. Control animals injected with the inducible AAVs but not given MFP, had background levels of IFN-α. These data suggest that MSC can be isolated, expanded in vitro, and infected with an AAV vector. This vector can confer a drug inducible secretion of a biologically active IFN-α, which appears to have similar biological properties to pharmacy grade Intron A. CML cell lines and patient samples treated with MSC-produced IFN-α are growth arrested, and the K562/BV173 CML cell lines are lethal when injected IV into nude mice. Using this mouse model circulating levels of human IFN-α were detected. Human MSC may also be detected using molecular, fluorescent, and immunohistochemical approaches.

### Example 12

### Harvesting, Culture, and Infection of MSC

Exemplary methods for harvesting, culture, and infection of MSC. Briefly, bone marrow aspirations or peripheral blood samples are harvested and rinsed once in PBS. The resulting culture is plated on tissue culture plastic in RPMI supplemented with 25% FCS. After 7 days, bone marrow cells are suspended by rubber policeman, and reacted with anti-sh2, sh3, sh4 antibodies (markers for MSC), after washing, a magnetic microbead reagent is reacted to bind the sh2,3,4 antibodies, and this mixture is passed over a magnetic enrichment column. After 15-18 days individual colonies grow out which are fibroblast-like in morphology, these are expanded for additional week. For infection, MSCs are rinsed once with PBS and then incubated with RPMI (200 μl) containing 1000-10,000 genomes of AAV βgal or AAV-IFN. Infection is allowed to proceed for 4 h and then fresh media containing 25% FCS is added. Forty-eight hours later cells are analyzed for βgal expression using X-gal histochemical staining or analysis with FACS utilizing CM-FDG, as in Marini et al. (1999). These AAV infected cells are expanded until adequate cell numbers are obtained. To induce IFN-α expression from AAV-infected MSC, cells are fed medium containing (10⁻⁷, 10⁻⁸, 10⁻⁹ M) MFP suspended in 0.1% ETOH. Eighteen hours later cells are washed once and fresh culture medium is added. Six to 24 h later, supernatant is collected and analyzed via Quantikine IFN-α ELISA (Biosource International Inc Camarillio Calif.).

### Example 13

### Detection of MSC

To detect MSC cells administered to an subject 3 techniques have been assembled that will provide sufficient sensitivity and allow for correct identification.

a. Membrane labeling of MSC: MSCs are cultured in media containing 33 μM SP-DIL or 50 μM PKH-26 for 10 min after which cells are washed three times and a sample is cytospun and membrane staining is confirmed under image analysis.

b. Immunohistochemical detection: AS-02 antibody purchased from (Sigma, St Louis, Mo.) is reacted with cytospun cells, frozen section or paraffin-embedded tissues. After washing, a horseradish peroxidase secondary anti-mouse IgG is reacted and after incubation and washing DDAB stain (Vectastain) is added and the reaction is terminated when a dark brown precipitate is formed.

c. PCRTM: human or mouse tissues are weighed (10 mg of wet tissue are used), and lysed in Trizol after isolation DNA is aliquoted and subjected to PCR™ using primers to βgal as described in Marini et al (1995).

### Example 14

### Implantation of Gene-Modified MSC or Direct IM Injection into Mouse Models

Implantation of gene-modified MSC or direct IM injection into mouse models: Briefly, 2×10⁶ to 1×10⁷ gene modified MSC is injected I.V. via tail vein into nu/nu or Balb/C/nu mice. Five mice /group are used and at 7 days, 4 weeks, 8 weeks, 12 weeks, to 6 months, mice are sacrificed, organs, and tissues harvested, and subjected to histology and X-gal staining. Additionally, the bone marrow from these mice is flushed, and cultured in vitro for another 5-7 days, and then stained for X-gal+cells. For analysis of IFN-α expression, 1×10¹⁰ vector G.E./mouse will be injected IM into the quadriceps muscle, and 10-20 days later 6 μg/mouse of MFP will be injected IP or added by gavage. Starting 48 h after MSC or AAV injection drug is given three times weekly, and blood samples (150-200 μl) obtained weekly and analyzed for IFN-α expression and quantity. Alternatively, after 5-8 weeks βgal+MSC 2 additional βgal assays are used, each more sensitive, a chemiluminescent βgal assay (Tropix Inc. Boston, Mass.), and RT-PCR.

### Example 15

### CML Models

Creation of CML models, and testing gene-modified MSCs: To create a CML model in which we can test IFN-α expressing MSC two CML cell lines are used which grow in a nu/nu or BalbC/nu mouse model. Briefly, K562 cells or BV173 are injected IV into respective mice (nu/nu and BalbC/nu). After a time period gene modified MSC, at 2×10⁶ cells/mouse, will be injected and the therapy initiated. As a control, mice injected with CML cell lines will also be injected daily with 3000 U of Intron A, this is a critical control in determining if cell-based therapy is more efficacious. Additional controls are mice injected with MSC expressing CMV-IFN-α (to determine maximum levels of IFN-α), and MSC expressing the inducible IFN-α, but not induced.

The best time after CML cell injection to begin induction of IFN-α is determined. Time points to evaluate are MFP activation directly after administration (1 or 2 days), or 10 days, 2 weeks where the MSC are already engrafting. Three endpoints are monitored: a) daily weight measurements are taken, as we observed these CML cell lines cause a wasting syndrome (decrease weight) before causing death, b) death as an endpoint (day of death) with is also taken, and c) in the event a moribund animal is observed this animal is sacrificed, blood recovered (for IFN-α expression levels, and to determine circulating levels of CML) and tissues, organs are subjected to pathological examination to determine MSC engraftment (using As-02, or Sh2,3,4 antibodies) and presence/absence of K562 or BV173 cells (using CD45 antibodies). In the event that a prolongation of mouse survival is observed mice may be multiply dosed with repeated administration of MSC (2×10⁶ MSC/mouse), this strategy will allow greater levels of MSC for engraftment. Alternatively, mice are engrafted with gene modified MSC (waiting 30 days after injection), and then inject CML cells.

Alternatively, fresh patient CD34+ CML cells are isolated using anti-CD34+ microbeads and MACS enrichment. These cells are then injected into 4 gy irradiated NOD-SCID mice (Thiesing et al., 2000). A one-time injection of 10 unit/mouse of stem cell factor is added to ensure engraftment. At various time points post injection PB is collect, spun down, and analyzed for CD45+(a marker for human myeloid cells), and CD33+. When each mouse expresses detectable CD45+ CML cells, these mice will then be utilized in the following studies. Alternatively, KU812 cells are grown in culture, rinsed in PBS, and 1×10⁷ cells/mouse injected subcutaneously (Ogura et al., 1990). Four weeks post injection, the tumor is resected, ground into a single cell suspension, and injected I.V. These cultured KU812 cells become tumorigenic and the mouse succumbs in 30 days. During this period, the gene-modified MSC are tested.

A second alternative, CML leukemia which is retrovirally transformed is used (Pear et al., 1998). Briefly, mouse cells are harvested, and infected with a bcr/abl expressing retroviral vector, these cells are then transplanted back into sygeneic mice, and within 30-45 days the mice are overwhelmed with bcr/abl expressing myeloid cells.

### Example 16

### Intravenous Injection of MSC-IFN

Sixteen SCID mice were divided into 4 groups (4 mice each). Mice from group 1 were not injected with tumor cells and serve as healthy controls. Groups 2, 3, 4 received 2×10⁶ MDA 231 breast carcinoma cells through tail vein on day 1 (dl). Animals remained either untreated (group 2) or started treatment on day 8. Treatments consist of 50,000 IU of β-IFN (IFN sc) injected subcutaneously every other day (group 3) or, four doses of 1×10⁶ MSCs transfected with β-IFN (MSC-IFN) injected intravenously through tail vein in weekly intervals (group 4). Animals were sacrificed on day 30 and lungs were photographed, weighted, and stained for hematoxillin/eosin (H&E). (FIG. 10)

The weight of the lungs in mice from the study described above and in FIG. 10 (means and SEM) were determined (FIG. 11). Intravenously injected MSC-IFN (group 4) significantly inhibited growth of MDA 231 metastasis in lungs (p=0.0073). In contrast β-IFN injected subcutaneously was not effective (p=0.14). There was also significant difference between lung weight of untreated mice with MDA 231 tumors in lungs and healthy animals with no tumors in lungs. It confirm that a lungs weight actually reflect tumor burden and can be used as measure of treatment efficacy in our model (see also FIG. 10).

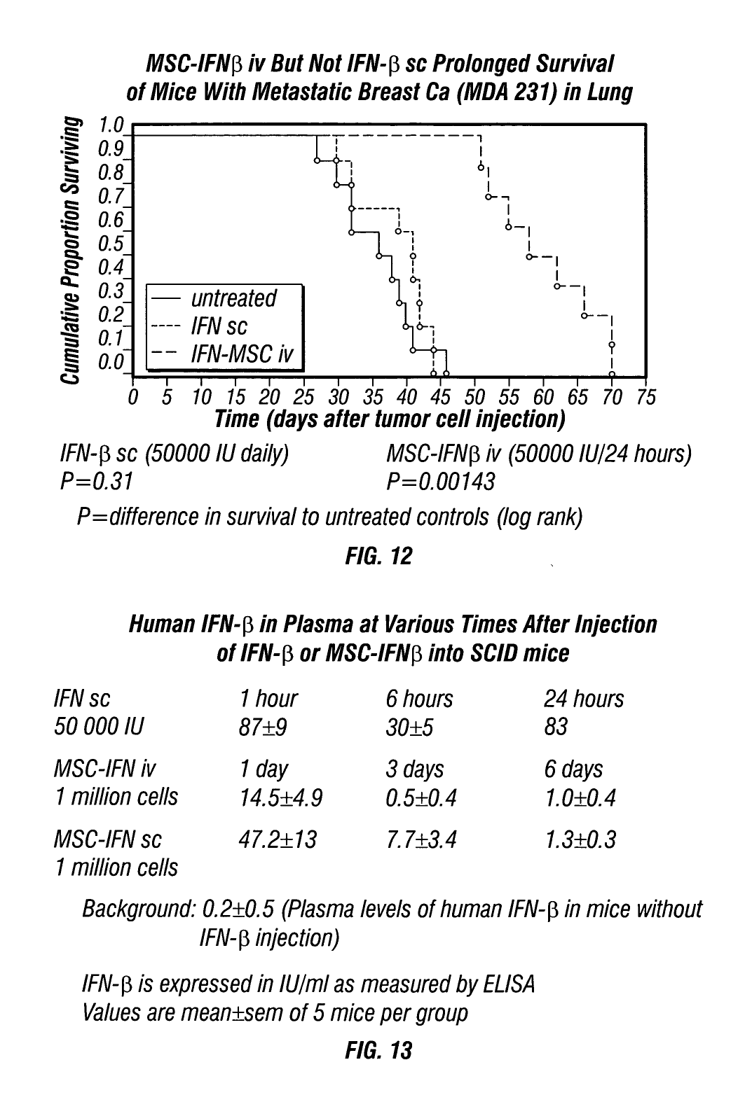

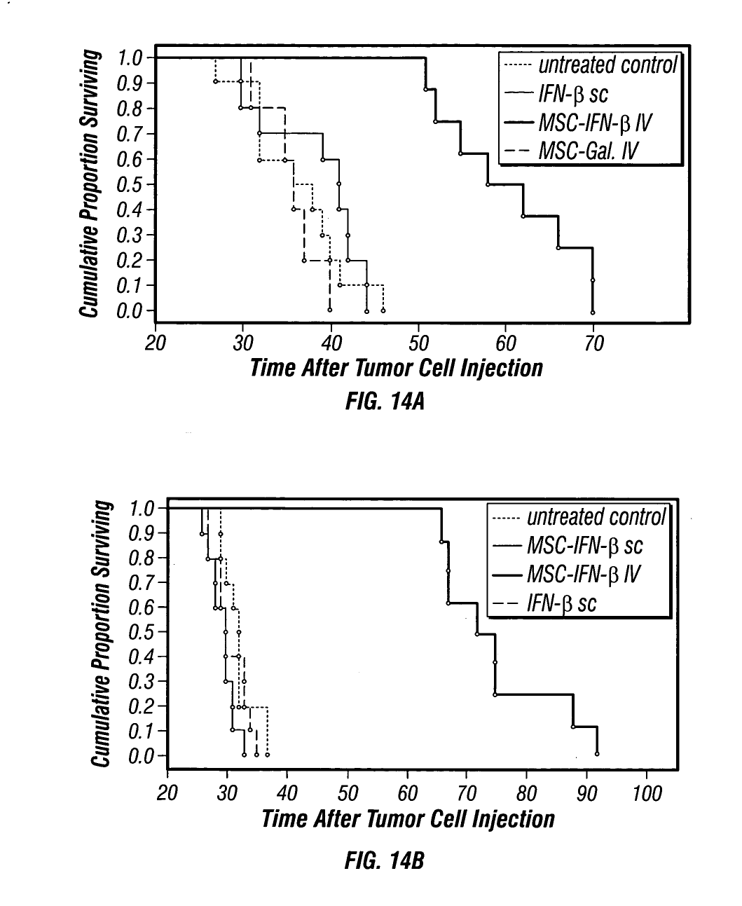

Survival of mice with MDA 231 tumors growing in lung and treated with MSC-IFN or IFN was also determined (FIG. 12). SCID mice (30 animals) were injected with 2×10⁶ MDA 231 breast carcinoma cells on day 1 and divided into 3 groups. Group 1 remained untreated and served as control. Group 2 were treated with 50,000 IU of IFN-beta injected subcutaneously (sc) every other day from day 8 until day 28. Group 3 was treated with four intravenous injections (iv) of 1×10⁶ MSC-IFN from day 8 until day 28 in weekly intervals. A single dose of MSC-IFN (1×10⁶ cells) produced approximately 50,000 IU of IFN during 24 h as determined in vitro before injection. Mice were followed until death. Survival of treated mice were compared to untreated controls by log rank test. MSC-IFN administered intravenously prolonged survival (p=0.00143) but, IFN was not effective (p=0.31).

IFN (50,000 IU in 200 μl of PBS with 0.1% FCS) injected subcutaneously (sc) or MSC-IFN (1×10⁶ MSC producing 50,000 IU of IFN in vitro) injected intravenously (iv) was administered to SCID mice with MDA 231 tumors growing in lungs. Then, plasma levels of human IFN in mice were determined at various time points. Tail veins were cut and blood collected into heparinized capillaries and immediately centrifuged to remove the blood cells. Plasma was stored at −80° C. until analysis. INF concentration was measured by ELISA kit (Fugirebio). Results indicate that MSC-IFN injected sc or iv lead to lower systemic (plasma) levels of IFN that subcutenous administration of IFN itself (FIG. 13).

The mouse model reflects the clinical situation regarding breast carcinoma insensitivity to systemic administration of IFN. In contrast to systemic administration of IFN, the MSC-IFN were highly effective in growth inhibition of MDA 231 breast carcinoma metastasis in lungs of SCID mice. The observed IFN levels in plasma of mice treated with MSC-IFN was below that observed in mice treated with IFN sc. Observed anti-tumor effect may not be related to systemic level of IFN and exemplifies the therapeutic benefit of MSC-IFN working through a paracrine effect of locally produced IFN.

### Example 17

### MSC Engraft in Tumors but not in Lungs or Other Organs of Healthy Animals

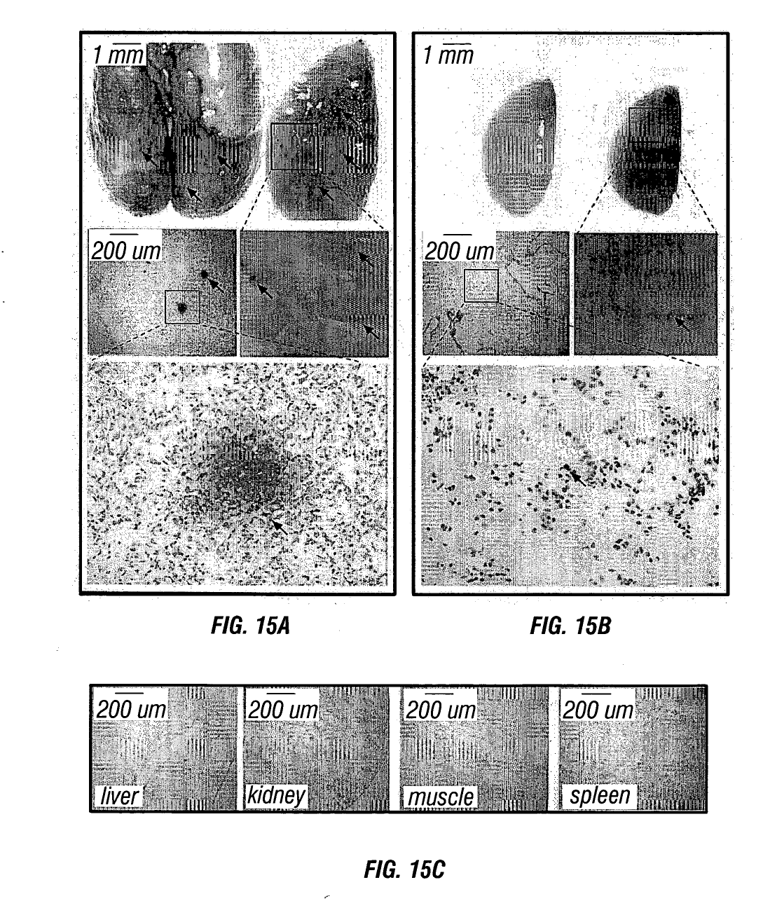

To confirm that intravenously injected MSC indeed engraft in tumors, mice were intravenously injected with three doses of 10⁶ MSC-Gal and their progeny traced by histochemical staining for X-Gal. One group of animals (n=5) had established MDA 231 metastases in their lungs, while another group (n=5) had no tumors. Histochemical staining was performed 14 days after the last dose of MSC-Gal (FIG. 15A-15C). Examination of tumors in lung showed numerous X-Gal positive cells (FIG. 15A). These cells formed colonies and became incorporated to the tumor architecture indicating that MSC could reach the extravascular space and contribute to the development of tumor connective stroma. It is likely that each colony originates from a single or very few MSC proliferating in-situ under the influence of signals from the surrounding microenvironment.

Moreover, when MSC-Gal were injected intravenously into healthy mice with no tumors (FIG. 15B), only very rare single X-Gal positive cells scattered in the lungs were found. These X-Gal positive cells showed no signs of proliferation or integration into the normal lung parenchyma. Similarly, no other examined organs (liver, spleen, kidney, muscle) in both groups of mice show signs of MSC-Gal integration into tissue (FIG. 15C) and only very rare single X-Gal positive cells were observed in the liver. These results confirmed a role of the tumor microenvironment for engraftment and proliferation of MSC in vivo.

### Example 18

### IFN-β and MSC-IFN-β Inhibit the Proliferation of OVAR-3, SKOV-3 and Hey Cells in Vitro

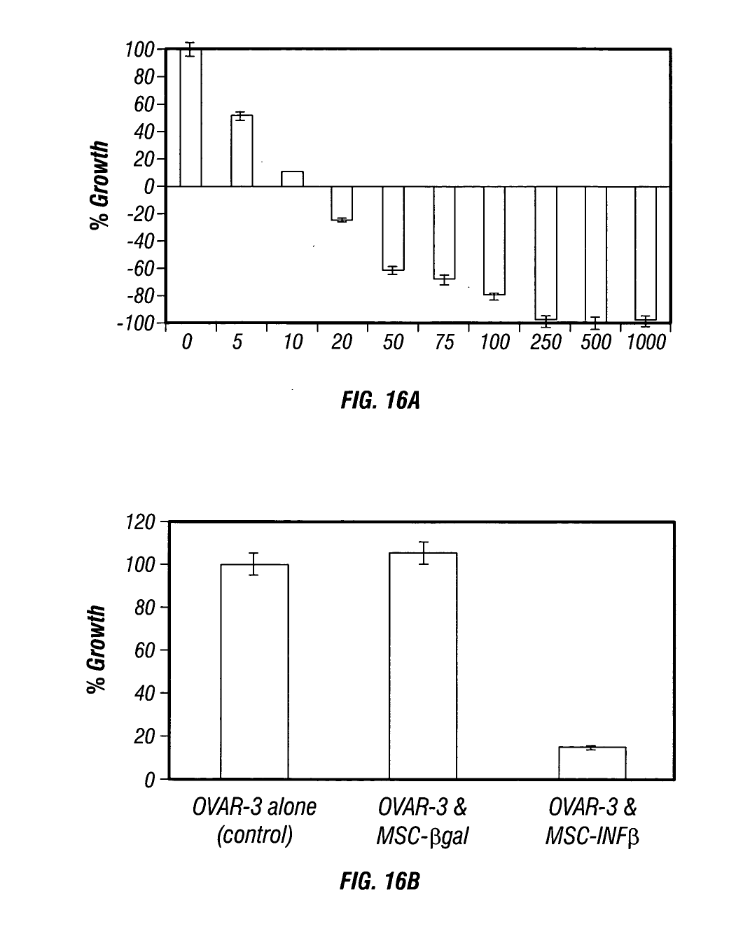

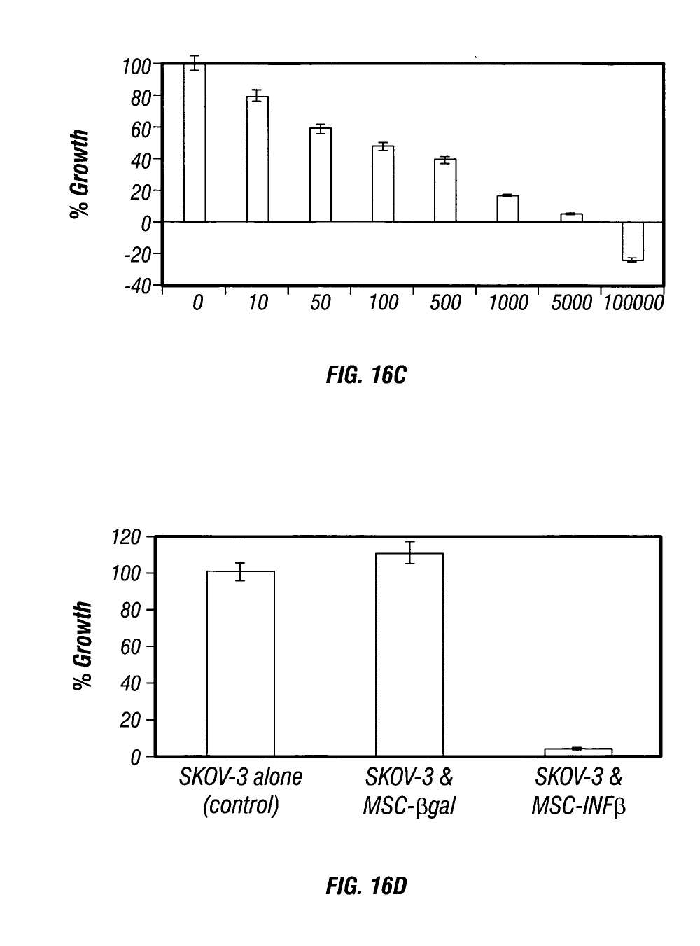

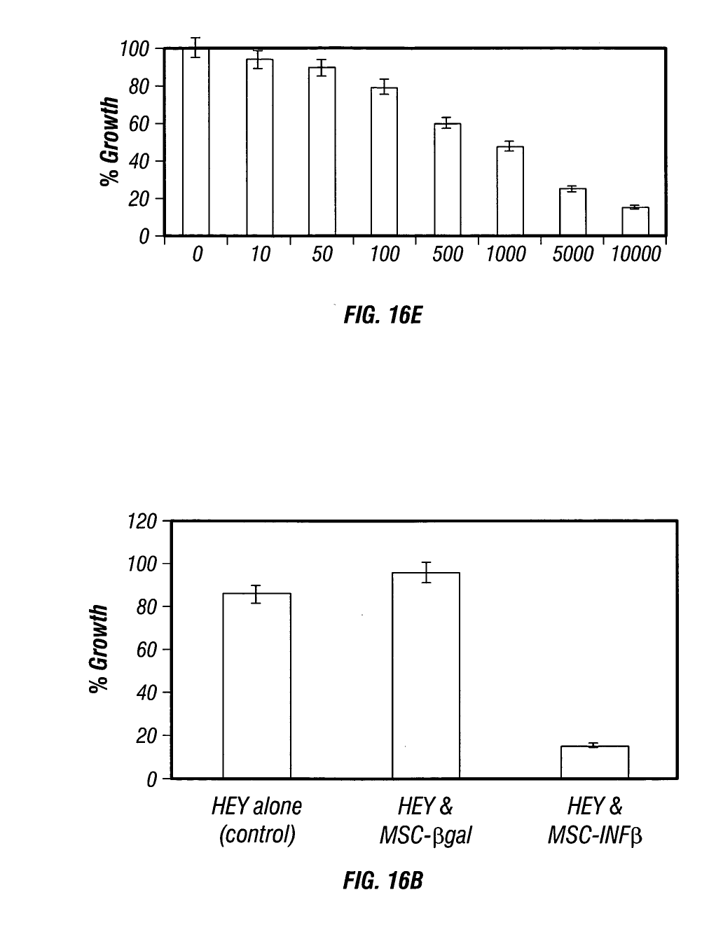

Both IFN-β and MSC-IFN-β inhibited the proliferation of OVAR-3, SKOV-3 and HEY ovarian carcinoma cells in vitro in a concentration dependent fashion. However, the OVAR-3 cells were the most sensitive to IFN-β (IC50 of 5 IU/ml), followed by the SKOV-3 cells (IC₅₀ of 100 IU/ml), then the Hey cells (IC₅₀ of 1000 IU/ml) (FIG. 16 A,C,E). Both OVAR-3 and SKOV-3 cells also showed evidence of cell death in addition to the growth inhibition. These results were consistent with the co-culture assay results, which are shown in FIG. 16 B,D, and F. In addition, as expected, normal MSCs (MSC-β gal) contributed to the growth of the tumor cells, which confirmed previous results.

### Example 19

### Concentration of IFN-β in Plasma after IFN-β and MSC-IFN-β Administration in Mice

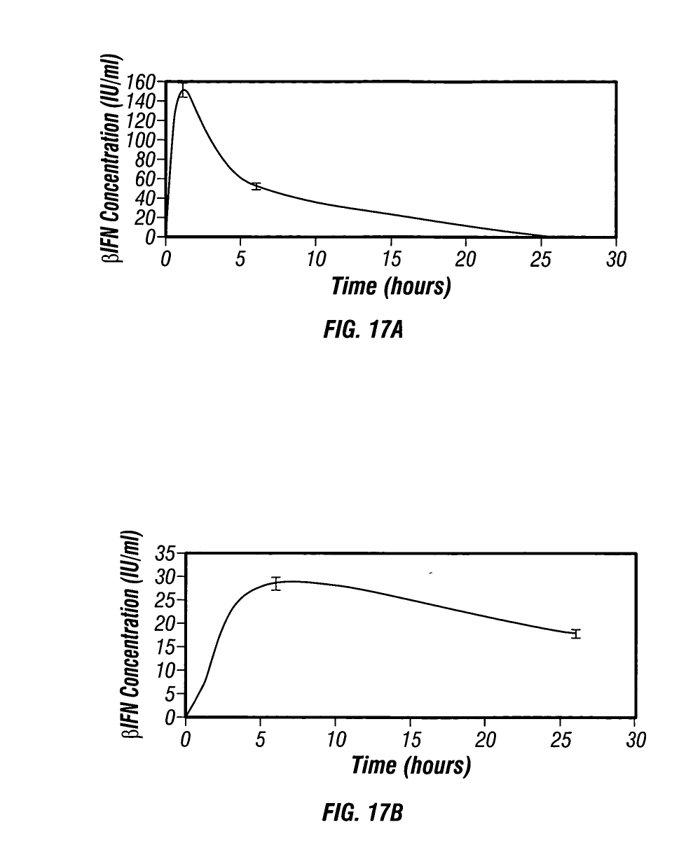

Recombinant IFN-β was rapidly broken down after intraperitoneal injection. Indeed, baseline levels were reached within 24 hours, proving that recombinant IFN-β cannot sustain levels systemically (FIG. 17). After the intraperitoneal injection of MSC-IFN-β however, detectable levels of IFN-β were found in the blood for at least 6 days. These data verify that MSC-IFN-β can sustain IFN-β production/levels in the blood.

### Example 20

### In VIVO Efficacy of MSC-IFN-β in OVAR-3, SKOV-3, and Hey Ovarian Carcinoma

The in vivo efficacy of MSC-IFN-β in OVAR-3, SKOV-3, and HEY ovarian carcinoma was tested using a SCID mice xenograft model. Tumors were established interperitoneally in mice by injecting tumor cells interperitoneally. Cells were injected in 1 ml of PBS (5×10⁶ OVAR-3, 6×10⁶ SKOV-3). After 15 days, tumors were established and treatments were begun. This consisted of five intraperitoneal injections of 5×10⁵ MSC-IFN-β given in weekly intervals. Control groups received either no treatment or five intraperitoneal injections of 5×10⁵ MSC-βgal in 1 ml of PBS. Additionally one group was given intraperitoneal injections of 40,000 IU of IFN-β in 1 ml of PBS every day from day 15 until day 48 to simulate, and compare MSC-IFN-β to conventional treatment with IFN-β. Animals were followed until death, and the difference in survival was analyzed by the log-rank test.

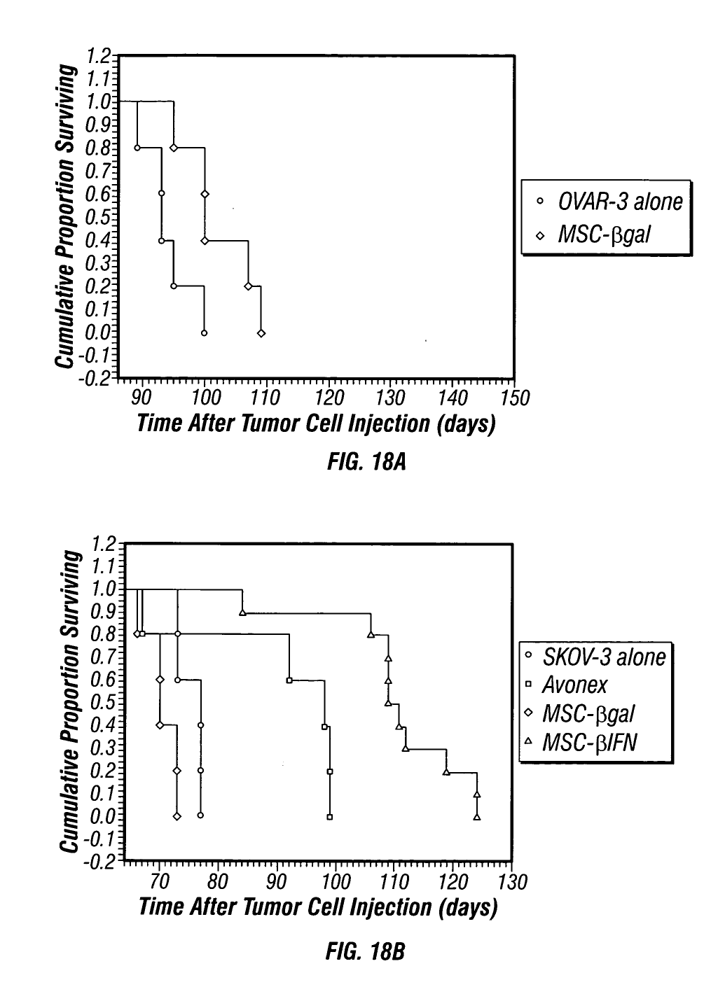

In OVAR-3 mice, MSC-IFN-β inhibited tumor growth and prolonged survival (p=) (FIG. 18) as compared to controls (p=). Systemically administered IFN-β however, also had an effect (p=) on survival, which is due to the increased sensitivity of these cells to IFN-β.

In SKOV-3 mice, MSC-IFN-β inhibited tumor growth and prolonged survival (p=0.000014) (FIG. 18) as compared to controls (p=0.0149). Systemically administered IFN-β however, also increased survival slightly as well (p=0.0343). This again can be attributed to the sensitivity of these cells to IFN-β as well.

### Example 21

### MSC Engraftment

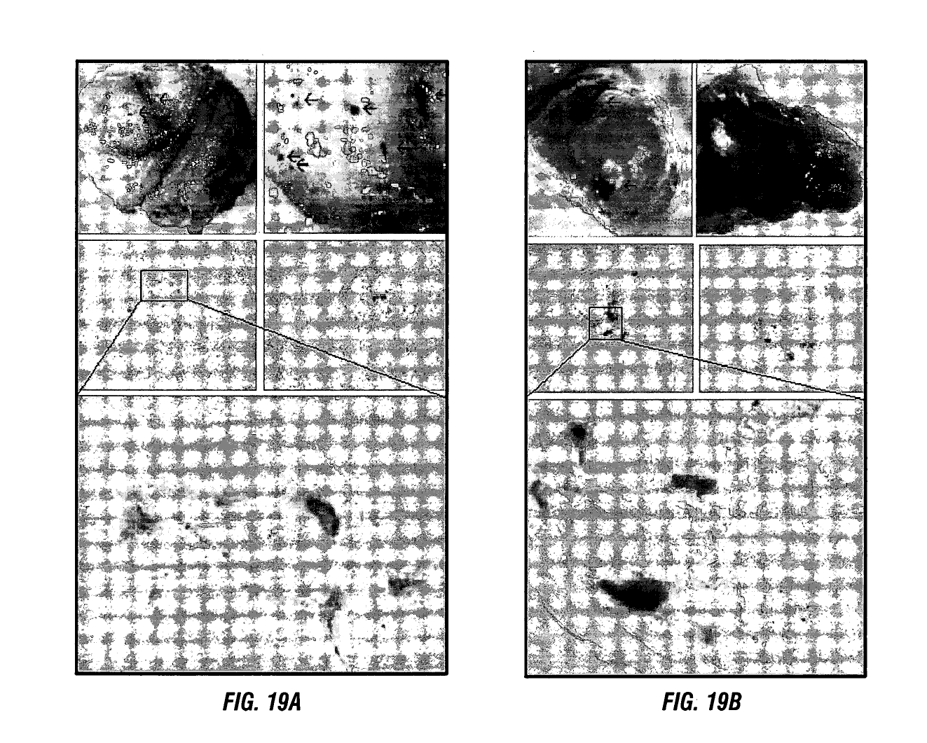

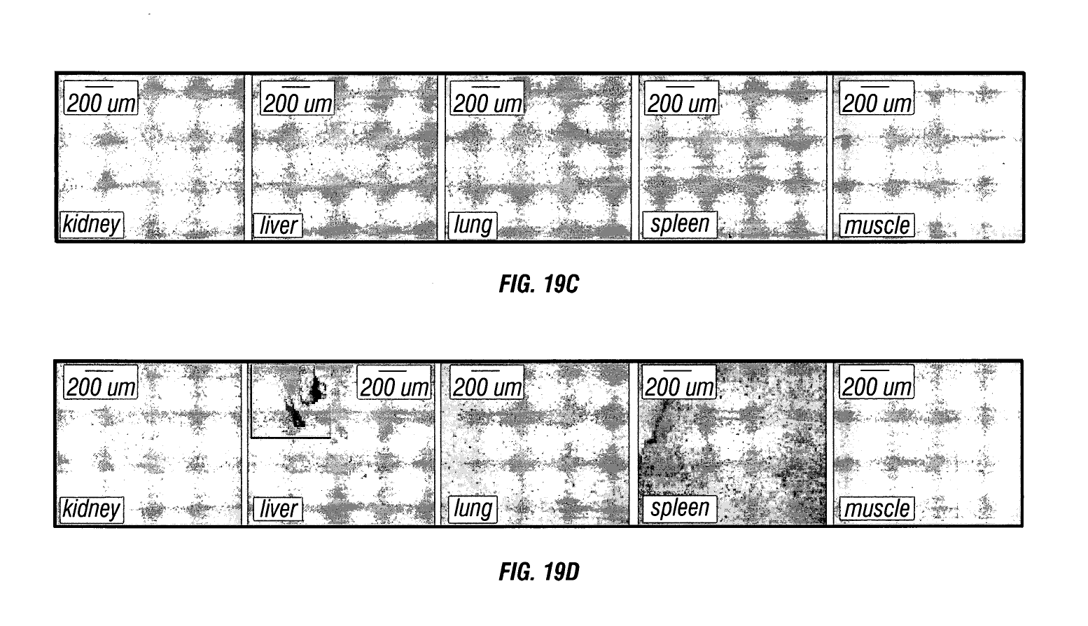

To confirm that intraperitoneally injected MSCs did engraft in tumors, mice were intravenously injected with five doses of 5×10⁵ MSC-βgal, and their progeny were traced histochemically with X-gal. Staining. One group of animals (n-5 for each cell type) had established intraperitoneal OVAR-3 or SVOV-3 carcinomas. Another group (n=3) had no tumors. Histochemical staining was performed 14 days after the last dose of MSC-βgal (FIG. 19). The tumors showed numerous X-gal positive cells that had formed colonies and become incorporated into the tumor architecture (FIG. 19A). It is very likely that each colony originated from a single or very few MSCs that proliferated under the influence of signals from surrounding microenvironment. However, when MSC-βgal was injected intraperitoneally into healthy mice with no tumors, only a very rare single X-gal positive cell was found (FIG. 19D). In addition, the MSCs showed no sign of proliferation or integration into normal tissue. Other organs (liver, spleen, kidney, muscle) in mice with ovarian carcinomas also did not show MSC-ogal engraftment (FIG. 19C) with only a very rare single X-gal positive cell in the liver. This confirmed the critical role of the tumor microenvironment for the successful engraftment of MSCs in vivo.

### Example 22

### Effects of IFNα and ST1571 on STI-Resistant KBM5 Cells

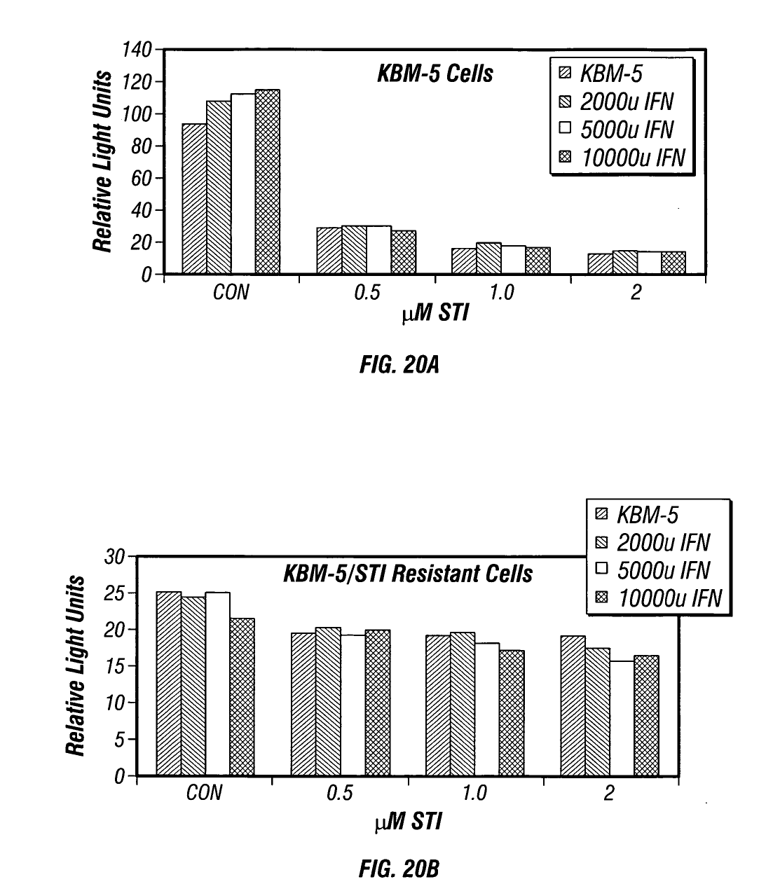

The effects of IFNA and ST1571 on STI-resistant KBM5 cells were studied. KBM-STI cells were confirmed to be resistant to ST1571, at least at the 0.5, 1.0, and 2.0 tmol concentrations of ST1571 tested, while KBM5 cells were highly sensitive. Recombinant IFNα (2,000, 5,000, and 10,000 U) had pronounced growth inhibitory effects on KBM5 cells, but not on KBM5-STI cells, and did not enhance the STI effect in vitro (FIG. 20A-20B). These in vitro data do not exclude significant in vivo effects of IFNα in STI-resistant CML, for example through the generation of cytotoxic, CML-specific T cells (Molldrem et al., 2000).

### Example 23

### Gene-Modified MSC in a Xenograft Model of CML

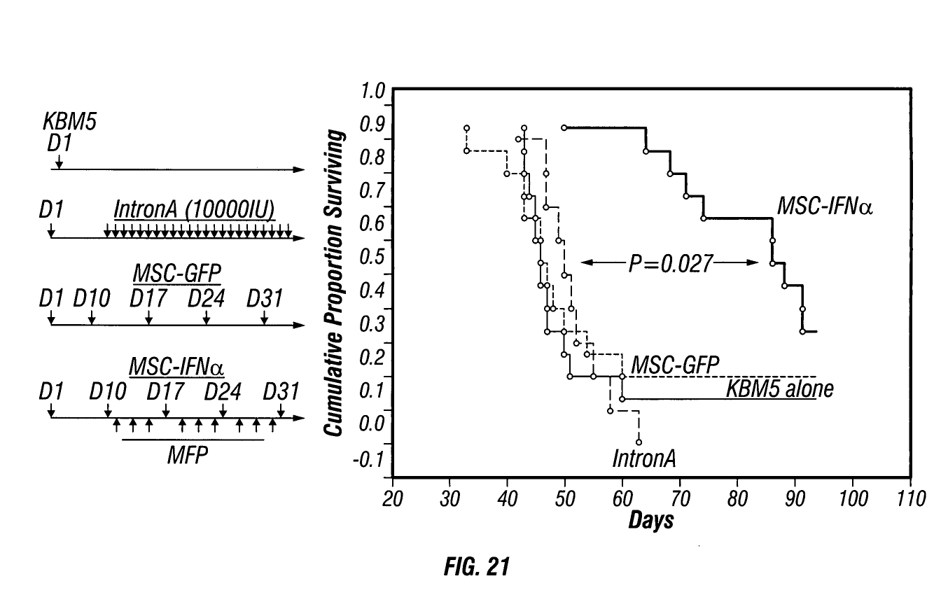

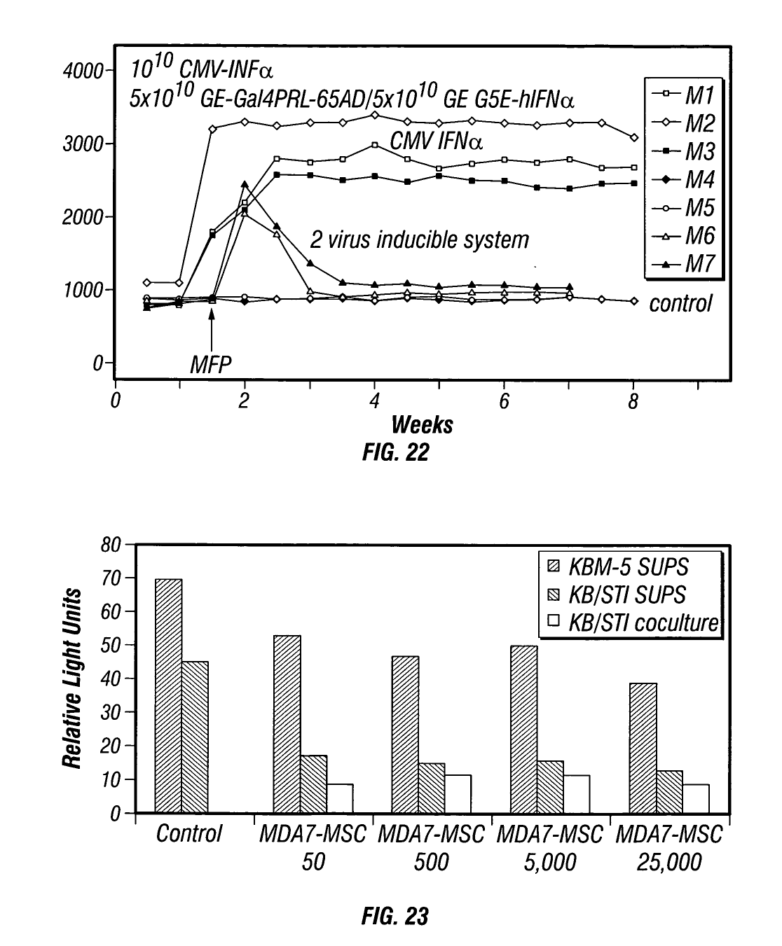

To initiate an in vivo model of CML, KBM5 BCR-ABL⁺ cell line were used (which establishes both extramedullary tumors and leukemia in the bone marrow of scid mice). KBM5 cells were derived from CML myeloid blast crisis cells that have been extensively characterized. A ST1571 resistant subline has been established. A dose of 2×10⁷ KBM5 cells results in death of SCID mice in 45-60 days. Mice injected with 2×10⁷ KBM5 cells where allowed to engraft for 10 days, then 2×10⁶ MSC expressing either GFP (control) or MFP-inducible INFα were injected into tail veins of mice and allowed to engraft. As controls, mice injected with KBM5 cells were also injected every other day with 1,000IU of recombinant IntronA. Three days after MSC injection mice received subcutaneous injections of MFP. As shown in FIG. 21, mice receiving KBM5 leukemia succumbed to disease between days 45-55. Upon necropsy, these mice had splenomegaly, and infiltration of lungs and bone marrow (data not shown) with KBM5 cells. KBM5 engrafted mice treated with 4 injection of GFP-MSC died at a similar rate as control mice, whereas 3 times weekly injections of IntronA extended mice survival by 2-to-5 days, a time period not statistically significant to controls. However in KBM5 engrafted mice treated with IFNα-MSC, a significant extension of survival was observed from 45 to 85 days, suggesting that MSC expressing INFα resulted in suppression of KBM5. The physical appearance of the mice and necropsy results confirmed this finding (data not shown). As an alternative to using MSC as a cell based therapy, AAV vectors were injected directly into the mouse quadriceps muscle, thereby allowing secretion of INFα systemically. This route of delivery by an intramuscular injection of 1010 GE AAV-CMV-INFα or 5×10¹⁰ GE of AAV-Gal4hPRL-65AD and 5×10¹⁰ GE of AAV-G5E-INFα into the quadriceps of mice were evaluated. Mice were bled weekly and the serum was analyzed for INFα expression using the Biosource ELISA kit. As shown in FIG. 22, circulating levels of hINFα were detected in the peripheral blood. In three animals injected with the constitutively expressing INFα (AAV-CMV-INFα), over 2600 pg/ml of hINFα was detected that required 2 weeks to reach maximum levels and this activity remained constant for the two months surveyed. Additionally, mice injected with the inducible AAV show a drug dependent induction of INFα (mice 6 and 7). Of note is that one application of MFP (given IP) results in a single peak of INFα activity that decays over a 7-day period. Control animals injected with the inducible AAV but not given MFP, had background levels of INFα. This method of IM injection of AAV-INFα results in high-level systemic expression of INFα.

### Example 24

### MDA7-MSC Preferentially Inhibit STI-Resistant KBM5 Cells

MSC were infected with a β-gal MDA7 adenovirus provided by Dr. Sunil Chada (Introgen, Houston, Tex.) at 50, 500, 5,000, and 25,000 MOI. All experiments were performed in duplicate supernatant from control and MDA7-MSC was obtained and added to KBM5 and KBM5-STI cells and assayed at 24 and 72 hours. Co-cultures of β-gal MDA7-MSC and control MSC with KBM5 (STI-sensitive) and KBM5-STI (STI-resistant) cells were also performed, with the MSC growing to near-confluency and 0.125×10⁶ cells/mL plated on the MSC in 12 well plates. All wells were assayed at 24 and 72 hours. As shown in FIG. 23, KBM5-STI resistant cells showed exquisite sensitivity to supernatant derived from MDA7-MSC and to co-culture, while the parental KBM5 cells appeared less sensitive. These results require confirmation, but suggest that MDA7 is effective against STI-resistant KBM5 cells. Importantly, no toxic effects of MDA7 were observed on MSC, whose growth was not affected.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### VIII. References

The following references, to the extent that they provide exemplary procedural or other details supplementary to those set forth herein, are specifically incorporated herein by reference.

U.S. Pat. No. 4,683,202

U.S. Pat. No. 4,797,368

U.S. Pat. No. 5,139,941

U.S. Pat. No. 5,359,046

U.S. Pat. No. 5,384,253

U.S. Pat. No. 5,486,359

U.S. Pat. No. 5,538,880

U.S. Pat. No. 5,550,318

U.S. Pat. No. 5,610,042

U.S. Pat. No. 5,698,443

U.S. Pat. No. 5,925,565

U.S. Pat. No. 5,928,906

U.S. Pat. No. 5,935,819

U.S. Pat. No. 5,994,136

U.S. Pat. No. 6,013,516

Almendro et al., J. Immunol., 157(12):5411-5421, 1996.

Anderson, Nature, 392(Suppl.):25-30, 1998

Angel et al., Cell, 49:729, 1987.

Angel et al., Mol. Cell. Biol., 7:2256, 1987.

Angel et al., Mol. Cell. Biol., 7:2256, 1987.

Atchison and Perry, Cell, 46:253, 1986.

Atchison and Perry, Cell, 48:121, 1987.

Ausubel et al., In: Current Protocols in Molecular Biology, John, Wiley & Sons, Inc, NY, 1994.

Bacigalupo et al., Blood, 98:3174, 2001.

Baichwal and Sugden, In: Gene Transfer, Kucherlapati (ed.), NY, Plenum Press, 117-148, 1986.

Banerji et al., Cell, 27(2 Pt 1):299-308, 1981.

Banerji et al., Cell, 33(3):729-740, 1983.

Bedzyk et al., J. Biol. Chem., 265(30):18615-18620, 1990.

Berkhout et al., Cell, 59:273-282, 1989.

Blagosklonny, Leukemia, 16:570-572, 2002.

Blanar et al, EMBO J., 8:1139, 1989.

Blomer et al., J. Virol., 71(9):6641-6649, 1997.

Bodine and Ley, EMBO J., 6:2997, 1987.

Boshart et al., Cell, 41:521, 1985.

Bosze et al., EMBO J., 5(7):1615-1623, 1986.

Braddock et al., Cell, 58:269, 1989.

Broxmeyer et al., J. Immunol., 131:1300-1305, 1983.

Bulla and Siddiqui, J. Virol., 62:1437, 1986.

Burbage et al., Leuk Res., 21(7):681-690, 1997.

Campbell and Villarreal, Mol. Cell. Biol., 8:1993, 1988.

Campere and Tilghman, Genes and Dev., 3:537, 1989.

Campo et al., Nature, 303:77, 1983.

Cao et al., J. Virol., 74(24):11456-11463, 2000.

Caplan, J. Orthop. Res, 9:641, 1991.

Carbonelli et al., FEMS Microbiol. Lett., 177(1):75-82, 1999.

Celander and Haseltine, J. Virology, 61:269, 1987.

Celander et al., J. Virology, 62:1314, 1988.

Chandler et al., Cell, 33:489, 1983.

Chandler et al., Proc. Natl. Acad. Sci. USA, 94(8):3596-3601, 1997.

Chang et al., Mol. Cell. Biol., 9:2153, 1989.

Chatteiee et al., Proc. Natl. Acad. Sci. USA, 86:9114, 1989.

Chaudhary et al., Proc. Natl. Acad. Sci. USA, 87(3):1066-1070, 1990.

Chen and Okayama, Mol. Cell Biol., 7(8):2745-2752, 1987.

Choi et al., Cell, 53:519, 1988.

Cobaleda et al., Blood, 95(3):1007-1013, 2000.

Cocea, Biotechniques, 23(5):814-816, 1997.

Coffey et al., Science, 282:1332-1334, 1998

Cohen et al., J. Cell. Physiol., 5:75, 1987.

Colter et al., Proc. Natl. Acad. Sci. USA, 97:3213, 2000.

Costa et al., Mol. Cell. Biol., 8:81, 1988.

Cotton et al., Proc. Natl. Acad. Sci. USA, 89(13):6094-6098, 1992.

Coupar et al., Gene, 68:1-10, 1988.

Cripe et al., EMBO J, 6:3745, 1987.

Culotta and Hamer, Mol. Cell. Biol., 9:1376, 1989.

Curiel, Nat. Immun., 13(2-3):141-64, 1994.

Dandolo et al., J. Virology, 47:55-64, 1983.

De Villiers et al., Nature, 312(5991):242-246, 1984.

Deans and Moseley, Exp. Hematol., 28(8):875-884, 2000.

Deschamps et al., Science, 230:1174-1177, 1985.

Dikici et al., Pediatr. Int., 43(6):577-580, 2001.

Dvorak, N. Engl. J. Med., 315:1650, 1986.

Edbrooke et al., Mol. Cell. Biol., 9:1908, 1989.

Edlund et al., Science, 230:912-916, 1985.

Einhom and Grander, J. Interferon Cytokine Res., 16:275, 1996.

Enright and McGlave, Oncology, 11(9):1295-1300, 1997.

Esmali et al., Am. J. Ophthalmol., 132(6):945-947, 2001.

European Application EPO 0273085

Fechheimer et al., Proc. Natl. Acad. Sci. USA, 84:8463-8467, 1987.

Feng and Holland, Nature, 334:6178, 1988.

Firak and Subramanian, Mol. Cell. Biol., 6:3667, 1986.

Foecking and Hofstetter, Gene, 45(1):101-105, 1986.

Fraley et al., Proc. Natl. Acad. Sci. USA, 76:3348-3352, 1979.

Fridenshteinet al., Tsitologiia, 10:557, 1968.

Friedmann, Science, 244:1275-1281, 1989.

Fujita et al., Cell, 49:357, 1987.

Garini et al., Am. J. Kidney Dis., 38(6):E35, 2001.

Ghosh and Bachhawat, In: Liver Diseases, Targeted Diagnosis and Therapy Using Specific Receptors and Ligands. Wu et al. (eds.), Marcel Dekker, NY, 87-104, 1991.

Gilles et al., Cell, 33:717, 1983.

Gloss et al., EMBO J., 6:3735, 1987.

Gnant et al., Cancer Res., 59:3396-3403, 1999.

Godbout et al., Mol. Cell. Biol., 8:1169, 1988.

Goodboum and Maniatis, Proc. Natl. Acad. Sci. USA, 85:1447, 1988.

Goodboum et al., Cell, 45:601, 1986.

Gopal, Mol. Cell Biol., 5:1188-1190, 1985.

Graharn and Van der Eb, Virology, 52:456-467, 1973.

Greene et al., Immunology Today, 10:272, 1989

Gresser, Med. Oncol. Tumor Pharmacother., 3(3-4):223-230, 1986.

Grosschedl and Baltimore, Cell, 41:885, 1985.

Grunhaus and Horwitz, Sem. Virology, 200(2):535-546, 1992.

Guo et al., Leukemia, 16:2447-2453, 2002.

Hanahan and Weinberg, Cell, 100:57, 2000.

Haslinger and Karin, Proc. Natl. Acad. Sci. USA, 82:8572, 1985.

Hatzakis et al., J. Interferon Cytokine Res., 21(10):861-869, 2000.

Hauber and Cullen, J. Virology, 62:673, 1988.

Heise et al., Nat. Med., 3:639-645, 1997.

Hen et al., Nature, 321:249, 1986.

Hensel et al., Lymphokine Res., 8:347, 1989.

Herr and Clarke, Cell, 45:461, 1986.

Hertzog et al., Mol. Reprod. Dev., 39:226, 1994.

Hirochika et al., J. Virol., 61:2599, 1987.

Hirsch et al., Mol. Cell. Biol., 10:1959, 1990.

Holbrook et al., Virology, 157:211, 1987.

Horlick and Benfield, Mol. Cell. Biol., 9:2396, 1989.

Horwich et al., J. Virol., 64:642-650, 1990.

Horwitz et al., Nature Med., 5:309, 1999.

Huang et al., Cell, 27:245, 1981.

Hug et al., Mol Cell. Biol., 8:3065, 1988.

Hwang et al., Mol. Cell. Biol., 10:585, 1990.

Imagawa et al., Cell, 51:251, 1987.

Imbra and Karin, Nature, 323:555, 1986.

Imler et al., Mol. Cell. Biol, 7:2558, 1987.

Imperiale and Nevins, Mol. Cell. Biol., 4:875, 1984.

Inouye and Inouye, Nucleic Acids Res., 13:3101-3109, 1985.

Jakobovits et al., Mol. Cell. Biol., 8:2555, 1988.

Jameel and Siddiqui, Mol. Cell. Biol., 6:710, 1986.

Jaynes et al., Mol. Cell. Biol., 8:62, 1988.

Jiang et al., Oncogene, 11:2477-2486, 1995.

Jiang et al., Proc. Natl. Acad. Sci. USA, 93:9160-9165, 1996.

Johansson et al., Cell, 96:25, 1999.

Johns et al., J. Natl. Cancer. Inst., 84:1185, 1992.

Johnson et al., Mol. Cell. Biol., 9:3393, 1989.

Kadesch and Berg, Mol. Cell. Biol., 6:2593, 1986.

Kaneda et al., Science, 243:375-378, 1989.

Kantarjian et al., Ann. Intern. Med., 122:254-261, 1995.

Kantaijian et al., Clin Cancer Res., 9:160-166, 2003.

Karin et al., Mol. Cell. Biol., 7:606, 1987.

Katinka et al., Cell, 20:393, 1980.

Kato et al, J. Biol. Chem., 266:3361-3364, 1991.

Kawamoto et al., Mol. Cell. Biol., 8:267, 1988.

Kelleher and Vos, Biotechniques, 17(6):1110-1117, 1994.

Kiledjian et al., Mol. Cell. Biol., 8:145, 1988.

Klamut et al., Mol. Cell. Biol., 10:193, 1990.

Klein et al., Nature, 327:70-73, 1987.

Koch et al., Mol. Cell. Biol., 9:303, 1989.

Kolokol'tsov et al. Bull. Exp. Biol. Med., 132(1):686-688, 2001.

Konopleva et al., Blood, 93(5):1668-1676, 1999.

Kozlowski et al., J. Natl. Cancer. Inst., 72:913, 1984.

Kraus et al., FEBS Lett, 428(3):165-70, 1998.

Kriegler and Botchan, In: Eukaryotic Viral Vectors, Gluzman (Ed.), Cold Spring Harbor, Cold Spring Harbor Laboratory, NY, 1982.

Kriegler and Botchan, Mol. Cell. Biol., 3:325, 1983.

Kriegler et al., Cell, 38:483, 1984.

Kriegler et al., Cell, 53:45, 1988.

Kuhl et al., Cell, 50:1057, 1987.

Kuniyasu et al., Am. J. Pathol., 157:1523, 2000.

Kuniyasu et al., Int. J. Oncol., 19(4):681-685, 2001.

Kuznetsov, et al., Haematol., 97:561, 1997.

Lareyre et al., J. Biol. Chem., 274(12):8282-90, 1999.

Larsen et al., Proc. Natl. Acad. Sci. USA., 83:8283, 1986.

Laspia et al., Cell, 59:283, 1989.

Latimer et al., Mol. Cell. Biol., 10:760, 1990.

Lattime et al., Semin. Oncol., 23:88-100, 1996.

Laughlin et al., J. Virol., 60(2):515-524, 1986.

Le Bon et al., Immunity, 14:461, 2001.

1e Coutre et al., Blood, 95(5):1758-1766, 2000.

Lebkowski et al., Mol. Cell Biol., 8(10):3988-3996, 1988.

Lee et al., DNA Cell Biol., 16(11):1267-1275, 1997.

Lee et al., Nature, 294:228, 1981.

Lee et al., Nucleic Acids Res., 12:4191-4206, 1984.

Levenson et al., Hum. Gene Ther., 9(8):1233-1236, 1998.

Levinson et al., Nature, 295:79, 1982.

Lidor et al., Am. J. Obstet. Gynecol., 177(3):579-585, 1997.

Liechty et al., Nat. Med., 6(11):1282-1286, 2000.

Lin et al., Mol. Cell. Biol., 10:850, 1990.

Luria et al., EMBO J., 6:3307, 1987.

Lusky and Botchan, Proc. Natl. Acad. Sci. USA, 83:3609, 1986.

Lusky et al., Mol. Cell. Biol. 3:1108, 1983.

Macejak and Sarnow, Nature, 353:90-94, 1991.

Majors and Varmus, Proc. Nat'l Acad. Sci. USA, 80:5866, 1983.

Mann et al., Cell, 33:153-159, 1983.

Marini et al., Clin. Cancer Res., 5(6):1557-1568, 1999.

Marini et al., Gene Ther., 2(9):655-659, 1995.

Marini et al., Hum. Gene Ther., 6(9):1215-1223, 1995.

Masuda et al., Mol. Cell. Biol., 17:2066-2075, 1997.

McLaughlin et al., J. Virol., 62(6):1963-1973, 1988.

McNeall et al., Gene, 76:81, 1989.

Mhashilkar et al., Mol Med., 7:271-282, 2001.

Miksicek et al., Cell, 46:203, 1986.

Miller et al., Am. J. Clin. Oncol., 15(3):216-221, 1992.

Molldrem et al., Nat. Med., 6:1018-1023, 2000.

Moravcova et al., Eur. J. Haematol., 64(2):135-136, 2000.

Mordacq and Linzer, Genes and Dev., 3:760, 1989.

Moreau et al., Nucl. Acids Res., 9:6047, 1981.

Muesing et al., Cell, 48:691, 1987.

Muzyczka, Curr. Top Microbiol. Immunol., 158:97-129, 1992.

Nabel et al., Science, 244(4910):1342-1344, 1989.

Naldini et al., Science, 272(5259):263-267, 1996.

Ng et al., Nuc. Acids Res., 17:601, 1989.

Nicolas and Rubenstein, In: Vectors: A survey of molecular cloning vectors and their uses, Rodriguez and Denhardt (Eds.), Stoneham: Butterworth, 493-513, 1988.

Nicolau and Sene, Biochim. Biophys. Acta, 721:185-190, 1982.

Nicolau et al., Methods Enzymol., 149:157-176, 1987.

Nomoto et al., Gene, 236(2):259-71, 1999.

Ogura et al., Cancer Res., 50(16):5102-6, 1990.

Ondeket al., EMBO J., 6:1017, 1987.

Ornitz et al., Mol. Cell. Biol., 7:3466, 1987.

Palmiter et al., Nature, 300:611, 1982.

Paskind et al., Virology, 67:242-248, 1975.

PCT Application WO 94/09699

Pear et al., Blood, 92(10):3780-3792, 1998.

Pech et al., Mol. Cell. Biol., 9:396, 1989.

Pelletier and Sonenberg, Nature, 334:320-325, 1988.

Perales et al., Proc. Nat. Acad. Sci. USA, 91:4086-4090, 1994.

Perez-Stable and Constantini, Mol. Cell. Biol., 10:1116, 1990.

Picard and Schaffner, Nature, 307:83, 1984.

Pinkert et al., Genes and Dev., 1:268, 1987.

Pittenger et al., Science, 284:143, 1999.

Ponta et al., Proc. Natl. Acad. Sci. USA, 82:1020, 1985.

Porton et al., Mol. Cell. Biol., 10:1076, 1990.

Potter et al., Proc. Natl. Acad. Sci. USA, 81:7161-7165, 1984.

Qin et al., Mol. Ther., 4:356, 2001.

Queen and Baltimore, Cell, 35:741, 1983.

Quinn et al., Mol. Cell. Biol., 9:4713, 1989.

Redondo et al., Science, 247:1225, 1990.

Reisman and Rotter, Mol. Cell. Biol., 9:3571, 1989.

Remington's Pharmaceutical Sciences” 15th Edition, chapter 33, in particular pages 624-652.

Remington's Pharmaceutical Sciences, 18th Ed. Mack Printing Company, 1990,

Resendez Jr. et al., Mol. Cell. Biol., 8:4579, 1988.

Ridgeway, In: Vectors: A survey of molecular cloning vectors and their uses, Stoneham: Butterworth, 467-492, 1988.

Ripe et al., Mol. Cell. Biol., 9:2224, 1989.

Rippe et al., Mol. Cell Biol., 10:689-695, 1990.

Rittling et al., Nuc. Acids Res., 17:1619, 1989.

Rosen et al., Cell, 41:813, 1988.

Roux et al., Proc. Natl. Acad. Sci. USA, 86:9079-9083, 1989.

Saalbach et al., J. Invest. Dermatol., 106:1314, 1996.

Saeki et al., Gene Ther., 7:2051-2057, 2000.

Saeki et al., Oncogene, 21:4558-4566, 2002

Sakai et al., Genes and Dev., 2:1144, 1988.

Salmon, J. Interferon Cytokine Res., 16:759, 1996.

Sambrook et al., In: Molecular Cloning: A Laboratory Manual, 2nd Ed., Cold Spring Harbor Press, Cold Spring Harbor, N.Y., 2001.

Satake et al., J. Virology, 62:970, 1988.

Schafffier et al., J. Mol. Biol., 201:81, 1988.

Searle et al., Mol. Cell. Biol., 5:1480, 1985.

Sharp and Marciniak, Cell, 59:229, 1989.

Shaul and Ben-Levy, EMBO J, 6:1913, 1987.

Sherman et al., Mol. Cell. Biol., 9:50, 1989.

Sleigh and Lockett, J. EMBO, 4:3831, 1985.

Spalholz et al., Cell, 42:183, 1985.

Spandau and Lee, J. Virology, 62:427, 1988.

Spandidos and Wilkie, EMBO J., 2:1193, 1983.

Stephens and Hentschel, Biochem. J., 248:1, 1987.

Strander, Adv. Cancer Res., 46:1-265, 1986.

Stuart et al., Nature, 317:828, 1985.

Sullivan and Peterlin, Mol. Cell. Biol., 7:3315, 1987.

Swartzendruber and Lehman, J. Cell. Physiology, 85:179, 1975.

Takebe et al., Mol. Cell. Biol., 8:466, 1988.

Talpaz et al., J. Haematol., 79(1):38-41, 1991.

Tavernier et al., Nature, 301:634, 1983.

Taylor and Kingston, Mol. Cell. Biol., 10:165, 1990a.

Taylor and Kingston, Mol. Cell. Biol., 10:176, 1990b.

Taylor et al., J. Biol. Chem., 264:15160, 1989.

Temin, In: Gene Transfer, Kucherlapati (Ed.), NY, Plenum Press, 149-188, 1986.

Thiesen et al., J. Virology, 62:614, 1988.

Thiesing et al., Blood, 96(9):3195-3199, 2000.

Toda et al., Hum. Gene Ther., 9:2177-2185, 1998.

Tratschin et al., Mol. Cell. Biol., 4:2072-2081, 1984.

Treisman, Cell, 42:889, 1985.

Tronche et al., Mol. Biol. Med., 7:173, 1990.

Trudel and Constantini, Genes and Dev. 6:954, 1987.

Tsumaki et al., J. Biol. Chem., 273(36):22861-4, 1998.

Tur-Kaspa et al., Mol. Cell Biol., 6:716-718, 1986.

Tyndell et al., Nuc. Acids. Res., 9:6231, 1981.

Vannice and Levinson, J. Virology, 62:1305, 1988.

Vasseur et al., Proc Natl. Acad. Sci. USA, 77:1068, 1980.

Wagner et al., Proc. Natl. Acad. Sci. USA, 87(9):3410-3414, 1990.

Wang and Calame, Cell, 47:241, 1986.

Weber et al., Cell, 36:983, 1984.

Weinberger et al. Mol. Cell. Biol., 8:988, 1984.

Weissman, Cell, 100:157, 2000.

Wilson et al., Science, 244:1344-1346, 1989.

Winoto and Baltimore, Cell, 59:649, 1989.

Wong et al., Gene, 10:87-94, 1980.

Woodbury, et al., J. Neurosci. Res., 61:364, 2000.

Wu and Wu, Adv. Drug Delivery Rev., 12:159-167, 1993.

Wu and Wu, Biochemistry, 27:887-892, 1988.

Wu and Wu, J. Biol. Chem., 262:4429-4432, 1987.

Wu et al., Biochem. Biophys. Res. Commun., 233(1):221-226, 1997.

Yang et al., Proc Natl. Acad. Sci. USA, 87:9568-9572, 1990.

Yutzey et al. Mol. Cell. Biol., 9:1397, 1989.

Zhao-Emonet et al., Biochim. Biophys. Acta, 1442(2-3):109-119, 1998.

Zufferey et al., Nat. Biotechnol., 15(9):871-875, 1997.

What is claimed is:

1. A composition comprising a stromal cell precursor that is genetically modified to produce an anti-cancer agent.

2. The composition of claim 1, wherein said anti-cancer agent is a cytokine, a hormone, an extracellular matrix component, an enzyme, a signaling molecule, an anti-angiogenic polypeptide, or an oncolytic virus.

3. The composition of claim 1, wherein said genetically modified stromal cell precursor produces IFN-α or IFN-β.

4. The composition of claim 1, wherein said therapeutic agent is secreted from said genetically modified stromal cell precursor.

5. The composition of claim 1, wherein said therapeutic agent is expressed on a cell surface of said genetically modified stromal cell precursor.

6. The composition of claim 1, further comprising a pharmaceutically acceptable carrier.

7. The composition of claim 1, wherein said stromal cell precursor is genetically modified by genomic integration of an expression cassette.

8. The composition of claim 1, wherein said stromal cell precursor is genetically modified by episomal expression vector.

9. The composition of claim 8, wherein said expression vector is a viral expression vector.

10. An anti-cancer composition comprising a stromal cell precursor engineered to express an anti-cancer gene product, wherein said stromal cell precursor preferentially localizes in an area of an organism undergoing hyperproliferative cell growth.

11. The composition of claim 10, wherein said stromal cell precursors engraft in a tumor.

12. The composition of claim 10, wherein said anti-cancer gene product is toxic when administered intravascularly.

13. The composition of claim 10, wherein said anti-cancer gene product is secreted from said stromal cell precursor.

14. The composition of claim 10, wherein said anti-cancer gene product is expressed on the cell surface of said stromal cell precursor.

15. The composition of claim 10, further comprising a pharmaceutically acceptable carrier.

16. The composition of claim 10, wherein said stromal cell precursor is genetically modified by genomic integration of an expression cassette.

17. The composition of claim 10, wherein said stromal cell precursor is genetically modified by episomal expression vector.

18. The composition of claim 10, wherein said anti-cancer gene product is IFN-α, IFN-β, IFN-γ or a combination thereof.

19. The composition of claim 17, wherein said expression vector is a viral expression vector.

20. A method for treatment of a subject with cancer comprising:
a) isolating stromal cell precursors from a donor;
b) propagating the isolated stromal cell precursors in vitro;
c) genetically modifying one or more of the isolated stromal cell precursors to express a therapeutic agent; and
d) introducing genetically modified stromal cell precursors into said subject.

21. The method of claim 20, wherein said genetically modified stromal cell precursors are introduced by injection.

22. The method of claim 21, wherein said genetically modified stromal cell precursors are introduced by intravascular injection.

23. The method of claim 21, wherein said genetically modified stromal cell precursors are introduced by intratumoral injection.

24. The method of claim 20, wherein said donor is said subject being treated.

25. The method of claim 20, wherein said donor is a heterologous donor.

26. A method for the delivery of a therapeutic agent to a patient comprising introducing a genetically modified stromal cell precursors to the patient.

27. The method of claim 26, wherein said patient is a cancer patient.

28. The method of claim 27, wherein said cancer patient has chronic myelogenous leukemia.

29. The method of claim 27, wherein said cancer patient has melanoma.

30. The method of claim 27, wherein said cancer patient has breast cancer.

31. The method of claim 27, wherein said cancer patient has ovarian cancer.

32. The method of claim 27, wherein said cancer patient has a brain cancer.

33. A method for reducing tumor growth comprising administering genetically modified stromal cell precursors.

34. The method claim 33, wherein said genetically modified stromal cell precursors are administered by injection.

35. The method of claim 34, wherein said injection is intravascular.

36. The method of claim 34, wherein said injection is intratumoral.

37. The method of claim 33, wherein said stromal cell precursors express INF-α or INF-β.

38. The method of claim 37, wherein said stromal cell precursors express INF-α.

39. The method of claim 37, wherein said stromal cell precursors express INF-β.

40. A method of reducing tumor burden in a patient with cancer comprising administering a genetically modified stromal cell precursors.

41. The method claim 40, wherein said genetically modified stromal cell precursors are administered by injection.

42. The method of claim 41, wherein said injection is intravascular.

43. The method of claim 41, wherein said injection is intratumoral.

44. The method of claim 40, wherein said stromal cell precursors express MNF-α or INF-β.

45. The method of claim 44, wherein said stromal cell precursors express INF-α.

46. The method of claim 44, wherein said stromal cell precursors express INF-β.

47. A method of treating metastatic cancer comprising administering a genetically modified stromal cell precursors.

48. The method claim 47, wherein said genetically modified stromal cell precursors are administered by injection.

49. The method of claim 48, wherein said injection is intravascular.

50. The method of claim 48, wherein said injection is intratumoral.

51. The method of claim 47, wherein said stromal cell precursors express INF-α or INF-β.

52. The method of claim 51, wherein said stromal cell precursors express INF-α.

53. The method of claim 51, wherein said stromal cell precursors express INF-β.

54. A method of rendering an inoperable tumor operable comprising administering a genetically modified stromal cell precursors.

55. The method claim 54, wherein said genetically modified stromal cell precursors are administered by injection.

56. The method of claim 55, wherein said injection is intravascular.

57. The method of claim 55, wherein said injection is intratumoral.

58. The method of claim 54, wherein said stromal cell precursors express INF-α or INF-β.

59. The method of claim 58, wherein said stromal cell precursors express INF-α.

60. The method of claim 58, wherein said stromal cell precursors express INF-β.

61. A method of increasing cancer patient survival comprising administering a genetically modified stromal cell precursors.

62. The method claim 61, wherein said genetically modified stromal cell precursors are administered by injection.

63. The method of claim 62, wherein said injection is intravascular.

64. The method of claim 63, wherein said injection is intratumoral.

65. The method of claim 61, wherein said stromal cell precursors express INF-α or INF-β.

66. The method of claim 65, wherein said stromal cell precursors express INF-α.

67. The method of claim 65, wherein said stromal cell precursors express INF-β.

68. A method of inhibiting hyperproliferative disease comprising administering a genetically modified stromal cell precursors.

69. The method claim 68, wherein said genetically modified stromal cell precursors are administered by injection.

70. The method of claim 69, wherein said injection is intravascular.

71. The method of claim 69, wherein said injection is intratumoral.

72. The method of claim 68, wherein said stromal cell precursors express INF-α or INF-β.

73. The method of claim 72, wherein said stromal cell precursors express INF-α.

74. The method of claim 72, wherein said stromal cell precursors express INF-β.

75. A method of engrafling a therapeutic cell in a tumor comprising:
a) isolating stromal cell precursors;
b) propagating the isolated stromal cell precursors in vitro;
c) genetically modifying one or more of the isolated stromal cell precursors to express a therapeutic agent; and
d) administering said genetically modified stromal cell precursors to a subject.

76. The method claim 75, wherein said genetically modified stromal cell precursors are administered by injection.

77. The method of claim 76, wherein said injection is intravascular.

78. The method of claim 77, wherein said injection is intratumoral.

79. The method of claim 75, wherein said stromal cell precursors express INF-α, INF-β, or a combination thereof.

80. The method of claim 79, wherein said stromal cell precursors express INF-α.

81. The method of claim 79, wherein said stromal cell precursors express INF-β.
